(19) United States
(12) Patent Application Publication
(10) Pub. No.: US 2011/0263435 A1
(43) Pub. Date: Oct. 27, 2011

(54) GENETIC MARKERS FOR BOAR TAINT

(75) Inventors: E. James Squires, Guelph (CA); Dominique Rocha, Oxon (GB); John Peacock, Thornhill (CA); Zhihong Lin, Chicago, IL (US); Nader Deeb, Franklin, KY (US)

(73) Assignee: UNIVERSITY OF GUELPH, GUELPH, ONTARIO (CA)

(21) Appl. No.: 12/160,253

(22) PCT Filed: Jan. 12, 2007
(86) PCT No.: PCT/US2007/060490

§ 371 Date: Sep. 10, 2009

Related U.S. Application Data
(60) Provisional application No. 60/758,825, filed on Jan. 13, 2006.

(30) Foreign Application Priority Data
Jan. 12, 2007 (US) PCT/US2007/060490

(51) Int. Cl. C12Q 1/68; C07H 21/00; G01N 27/447; C12N 9/10; H01J 49/40; G01N 33/559; C40B 20/00; C07K 14/00

(52) U.S. Cl. 506/2; 435/6.11; 536/23.5; 530/350; 536/23.2; 435/193; 250/282; 204/456

(57) ABSTRACT

Genetic markers are disclosed with a useful association with boar taint that can be used for screening and selection of pigs for those with more favorable boar taint characteristics associated with androstenone/skatole metabolism. Specific polymorphic alleles of the 3αHSD, 3βHSD, CYP17A1, CYP2A, CYP2E1, CYTB5, BAC-CT and/or SULT1A1 genes are disclosed for tests to screen pigs to determine those more likely to produce desired boar taint traits.

### FIELD OF THE INVENTION

This invention relates generally to the detection of genetic differences among animals. More particularly, the invention relates to genetic variation that is indicative of heritable phenotypes associated with preferred lower boar taint characteristics. Methods and compositions for use of specific genes, genetic markers and chromosomal regions associated with the variation in boar taint, in genotyping of animals and selection are also disclosed.

### BACKGROUND OF THE INVENTION

Researchers have found that quantitative trait phenotypes are continuously distributed in natural populations, due to segregation of alleles at multiple genes in different regions. These quantitative trait loci (QTL) combined with differences in environmental sensitivity of QTL alleles affect the phenotypes. Determining the genetic and environmental basis of variation for quantitative traits is important for human health, agriculture, and the study of evolution. But, complete genetic dissection of quantitative traits is currently feasible only in genetically tractable and well characterized model systems. (Mackay, Nat. Rev. Genet. 2:11-20 (2001); Wright et al., Genome Biol. 2:2007.1-2007.8 (2001)). For example, the number of genes involved in quantitative genetic variation is not known, the number and effects of individual alleles at these genes, or the gene action is also generally unknown. To date, genes and causal variants have been detected for very few quantitative traits. For example, such quantitative traits such as double-muscling in cattle (Grobet et al., Mamm. Genome 9:210-213 (1998)), alteration in fruit size (Frary et al., Science 289:85-88 (2000)), growth and performance traits in pigs (Kim et al., Mamm. Genome 11:131-135 (2000)), excess glycogen content in pig skeletal muscle (Ciobanu et al, Genetics 159:1151-1162 (2001)), improved meat quality (Milan et al., Science 288:1248-1251 (2000)), and increased ovulation and litter size in sheep (Wilson et al., Biol. Reprod. 64:1225-1235 (2001)). The effects of the mutations in the majority of these examples are so large that the phenotypes segregate almost as Mendelian traits.

To understand and exploit the genetics of complex quantitative traits, experimental populations derived from two lines differing widely for traits of interest have been successfully used in model species (Belknap et al., Behav. Genet. 23:213-222 (1993); Talbot et al., Nat. Genet. 21:305-308 (1999)), plants (Paterson et al., Nature 335:721-726 (1988)), and livestock (Andersson et al., Science 263:1771-1774 (1994)) to detect quantitative trait loci (QTL). These studies have succeeded in mapping QTL for which alleles differ in frequency between the parental populations, for example, between commercial agricultural cultivars and wild-type populations (Paterson et al., Nature 335:721-726 (1988); Andersson et al., Science 263:1771-1774 (1994)). In addition to understanding the architecture of quantitative traits, crosses involving agricultural species are also motivated by the potential to exploit variation within elite populations; commercial plant and animal populations are usually not based upon the same crosses that are used in the QTL detection studies but the power of linkage studies in line crosses is generally greater than that of studies within populations. In commercial pig breeding populations, for example, elite populations comprise closed outbred populations that have been subjected to selection over a number of generations to improve their commercial performance, whereas wild boar (Andersson et al., Science 263:1771-1774 (1994)) and Chinese Meishan (Walling et al. Anim. Genet. 29:415-424 (1998); De Koning et al, Genetics 152:1679-1690 (1999); De Koning et al, Proc. Natl. Acad. Sci. USA 97:7947-7950 (2000); Bidanel et al., Genet. Sel. Evol. 33:289-309 (2001)) populations have been often employed in QTL studies. The implicit hypothesis in many QTL studies using divergent lines is that knowledge of between-population genetic variation can be extrapolated to genetic variation in other populations or species. Segregation at QTL in commercial populations can be utilized by breeders through gene- or marker-assisted selection programs (e.g., Dekkers and Hospital, Nat. Rev. Genet. 3:22-32 (2002)).

Not all genes have an easily identifiable common functional variant that can be exploited in association studies, and in many gene cases researchers have identified only changes in individual nucleotides (i.e., single nucleotide polymorphisms (SNPs)) that have no known functional significance. Nevertheless, SNPs are potentially useful in narrowing a linkage region within a chromosome. In addition, SNPs may show a statistically significant association with a quantitative trait if located within or near that gene by virtue of linkage disequilibrium.

Significant markers or genes can then be included directly in the selection process. An advantage of the molecular information is that we can obtain it already at very young age of the breeding animal, which means that animals can be preselected based on DNA markers before the growing performance test is completed. This is a great advantage for the overall testing and selection system.

Polymorphisms hold promise for use as genetic markers in determining which genes contribute to multigenic or quantitative traits: suitable markers and suitable methods for exploiting those markers are beginning to be brought to bear on the genes related to boar taint.

Male pigs that are raised for meat production are usually castrated shortly after birth to prevent the development of off-odors and off flavors (boar taint) in the carcass. Boar taint is primarily due to high levels of either the 16-androstene steroids (especially 5α-androst-16-en-3-one) or skatole in the fat. Recent results of the EU research program AIR 3-PL94-2482 suggest that skatole contributes more to boar taint than androstenone (Bonneau, M., 1997).

Skatole is produced by bacteria in the hindgut which degrade tryptophan that is available from undigested feed or from the turnover of cells lining the gut of the pig (Jensen and Jensen, 1995). Skatole is absorbed from the gut and metabolized primarily in the liver (Jensen and Jensen, 1995). High levels of skatole can accumulate in the fat, particularly in male pigs, Skatole metabolism has been studied extensively in ruminants (Smith, et al., 1993), where it can be produced in large amounts by ruminal bacteria and results in toxic effects on the lungs (reviewed in Yost, 1989). Environmental and dietary factors affect skatole levels (Kjeldsen, 1993; Hansen et al., 1995) but do not sufficiently explain the reasons for the variation in fat skatole concentrations in pigs. Claus et al. (1994) proposed high fat skatole concentrations are a result of an increased intestinal skatole production due to the action of androgens and glucocorticoids. Lundström et al. (1994) reported a genetic influence on the concentrations of skatole in the fat, which may be due to the genetic control of the enzymatic clearance of skatole. The liver is the primary site of metabolism of skatole and liver enzymatic activities could be the controlling factor of skatole deposition in the fat. Bæk et al.(1995) described several liver metabolites of skatole found in blood and urine with the major being MII and MIII. MII, which is a sulfate conjugate of 6-hydroxyskatole (pro-MII), was only found in high concentrations in plasma of pigs which were able to rapidly clear skatole from the body, whereas high MIII concentrations were related to slow clearance of skatole. Thus the capability of synthesis of MII could be a major step in a rapid metabolic clearance of skatole resulting in low concentrations of skatole in fat and consequently low levels of boar taint.

Boar taint is caused by the accumulation of two main compounds in fat: 5α-androst-16-ene-3-one [androstenone]; (Patterson, 1968), and 3-methyl indole [skatole]; (Vold, 1970; Walstra and Marse, 1970) as described above. Androstenone is a male steroid pheromone that is produced from pregnenolone in the Leydig cells of the testis in a reaction catalyzed by cytochromes P450C17 and b5 (Meadus et al., 1993). Androstenone enters the systemic circulation by way of the spermatic vein and concentrates in the fat due to its hydrophobic properties (Davis and Squires, 1999). Genetic factors, sexual maturity, and possibly metabolism influence the rate of androstenone synthesis (Willeke, 1987). Thus factors which affect androstenone production or metabolism will also have effects on boar taint.

It can be seen from the foregoing that a need exists for identification of genetic variation associated with or in linkage disequilibrium with, several genomic regions, which may be used to improve economically beneficial characteristics in animals by identifying and selecting animals with the improved characteristics at the genetic level.

Another object of the invention is to identify genetic loci in which the variation present have quantitative effects on boar taint, a trait of interest to breeders.

Another object of the invention is to provide specific assays for determining the presence of such genetic variation in boar taint.

A further object of the invention is to provide a method of evaluating animals that increases accuracy of selection and breeding methods for pigs with lower boar taint.

Yet another object of the invention is to provide PCR amplification tests to greatly expedite the determination of presence of the marker(s) of such quantitative trait variation.

Additional objects and advantages of the invention will be set forth in part in the description that follows, and in part will be obvious from the description, or may be learned by the practice of the invention. The objects and advantages of the invention will be attained by means of the instrumentalities and combinations particularly pointed out in the appended claims.

### BRIEF SUMMARY OF THE INVENTION

The methods of the present invention comprise the use of nucleic acid markers genetically linked to loci associated with the presence of boar taint. The markers are used in genetic mapping of genetic material of animals to be used in and/or which have been developed in a breeding program, allowing for marker-assisted selection to identify or to move traits into elite germplasm. The invention relates to the discovery of genetic variation in genomic regions associated with or in linkage disequilibrium or otherwise genetically linked therewith that may be used to predict phenotypic traits in animals. According to an embodiment of the invention, several genes have been identified as major effect genes or as linked to such genes which are associated with differences in boar taint/skatole and/or androstenone metabolism. These include, 3α-hydroxysteroid dehydrogenase (3αHSD), 3β-hydroxysteroid dehydrogenase (3β-HSD), cytochrome P450 (CYP)17A1, cytochrome P450 (CYP)2A6, cytpchrome P450 (CYP)2E1, cytochrome B5, (CYTB5), sulfotransferase 1A1 (SULT1A1). In addition to these genes, 4 markers (223-226CP) were also identified as being linked to the SULT2A1 gene and were derived from a BAC end sequence GenBank Accession Number CT171681 (BAC-CT).

An embodiment of the invention is a method of identifying alleles of these genes that are associated with skatole/androstenone metabolism and boar taint comprising obtaining a tissue or body fluid sample from an animal; amplifying DNA present in said sample comprising a region of one or several of these genes; and detecting the presence of a polymorphic variant of said nucleotide sequences wherein said variant is associated with useful phenotypic variation in boar taint/skatole and/or androstenone metabolism.

Another embodiment of the invention is a method of determining a genetic marker which may be used to identify and select animals based upon their skatole and/or androstenone metabolism traits or propensity for boar taint comprising obtaining a sample of tissue or body fluid from said animals, said sample comprising DNA; amplifying DNA present in said sample in the region of one of these genes present in said sample from a first animal; determining the presence of a polymorphic allele present in said sample by comparison of said sample with a reference sample or sequence; correlating variability for skatole and/or androstenone metabolism in said animals with said polymorphic allele; so that said allele may be used as a genetic marker for the same in a given group, population, or species.

Yet anther embodiment of the invention is a method of identifying an animal for its propensity for boar taint, said method comprising obtaining a nucleic acid sample from said animal, and determining the presence of an allele characterized by a polymorphism in a gene sequence of 3αHSD, 3βHSD, CYP17A1, CYP2A, CYP2E1, CYTB5, BAC-CT and/or SULT1A1 sequence present in said sample, or a polymorphism in linkage disequilibrium therewith, said genotype being one which is or has been shown to be usefully associated with a trait indicative of skatole and/or androstenone metabolism and/or boar taint in a pig.

Additional embodiments are set forth in the Detailed Description of the Invention and in the Examples.

### DETAILED DESCRIPTION OF THE PREFERRED EMBODIMENT

Genetic markers closely linked to important genes may be used to indirectly select for favorable alleles more efficiently than direct phenotypic selection (Lande and Thompson 1990). Therefore, it is of particular importance, both to the animal breeder and to farmers who grow and sell animals as a cash crop, to identify, through genetic mapping, the quantitative trait loci (QTL) for various economically valuable traits such as low boar taint. Knowing the QTLs associated with these traits animal breeders will be better able to breed animals which possess genotypic and phenotypic characteristics. To achieve the objectives and in accordance with the purpose of the invention, as embodied and broadly described herein, the present invention provides the discovery of alternate chromosomal regions and genotypes which provide a method for genetically typing animals and screening animals to determine those more likely to possess favorable skatole and/or androstenone metabolism/boar taint traits or to select against animals which have alleles indicating less favorable skatole and/or androstenone metabolism/boar taint. As used herein a “favorable boar taint trait” means a useful improvement (increase or decrease) in one of any measurable indicia of boar taint including compounds involved in skatole, or androstenone metabolism different from the mean of a given animal, group, line, species or population which has the alternate allele form, so that this information can be used in breeding to achieve a uniform group, line or species, or population which is optimized for these traits. This may include an increase in some traits or a decrease in others depending on the desired characteristics. A useful improvement may or may not be statistically significant for a single SNP or trait or even for every population but may be still useful when used in combination with other markers or alternate groups of animals to show trends or haplotypes or variation within a single group.

The effect on a trait such as skatole and/or androstenone may be demonstrated specifically herein through the use of any of a number of particular identifiers, such as amount of androstenone, amount of skatole, but the invention is not so limited. As used herein the use of any particular indicia of the phenotypic traits of skatole metabolism, boar taint: e.g. amount of androstenone, amount of skatole, levels of enzymes, ligands, or substrates involved in skatole metabolism etc. shall be interpreted to include all indicia for which variability is associated with the disclosed allele with respect to skatole/androstenone metabolism or boar taint.

Methods for assaying for these traits generally comprises the steps 1) obtaining a biological sample from an animal; and 2) analyzing the genomic DNA or protein obtained in 1) to determine which allele(s) is/are present. Haplotype data which allows for a series of linked polymorphisms to be combined in a selection or identification protocol to maximize the benefits of each of these markers may also be used and are contemplated by this invention.

In another embodiment, the invention comprises a method for identifying genetic markers for skatole metabolism, androstenone metabolism and boar taint. Once a major effect gene has been identified as disclosed herein (3αHSD, 3βHSD, CYP17A1, CYP2A, CYP2E1, CYTB5, BAC-CT and/or SULT1A1), it is expected that other variation present in the same gene, allele or in sequences in useful linkage disequilibrium therewith may be used to identify similar effects on these traits without undue experimentation. The identification of other such genetic variation, once a major effect gene has been discovered, represents no more than routine screening and optimization of parameters well known to those of skill in the art and is intended to be within the scope of this invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison; in this case, the Reference sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.ncbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands.

For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I). The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involves no more than routine optimization of parameters using the teachings herein.

It is also possible to establish linkage between specific alleles of alternative DNA markers and alleles of DNA markers known to be associated with a particular gene (e.g., the genes discussed herein), which have previously been shown to be associated with a particular trait. Thus, in the present situation, taking one or both of the genes, it would be possible, at least in the short term, to select for animals likely to produce desired traits, or alternatively against animals likely to produce less desirable traits indirectly, by selecting for certain alleles of an associated marker through the selection of specific alleles of alternative chromosome markers. As used herein the term “genetic marker” shall include not only the nucleotide polymorphisms disclosed, but by any means of assaying for the protein changes associated with the polymorphism, be they linked genetic markers in the same chromosomal region, use of microsatellites, or even other means of assaying for the causative protein changes indicated by the marker and the use of the same to influence traits of an animal.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism: for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

The invention is intended to include the disclosed sequences as well as all conservatively modified variants thereof. The terms 3αHSD, 3βHSD, CYP17A1, CYP2A, CYP2E1, CYTB5, BAC-CT and/or SULT1A1 as used herein shall be interpreted to include conservatively modified variants which include the specific SNPs disclosed herein. The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His).

Those of ordinary skill in the art will recognize that some substitution will not alter the activity of the polypeptide to an extent that the character or nature of the polypeptide is substantially altered. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with meat quality/growth-like characteristics. When it is desired to alter the amino acid sequence of a polypeptide to create an equivalent, or a variant or portion of a polypeptide of the invention, one skilled in the art will typically change one or more of the codons of the encoding DNA sequence according to Table 1 (See infra). For example, certain amino acids may be substituted for other amino acids in a protein structure without appreciable loss of activity. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences, which encode said peptides without appreciable loss of their biological utility or activity. A degenerate codon means that a different three letter codon is used to specify the same amino acid. For example, it is well known in the art that the following RNA codons (and therefore, the corresponding DNA codons, with a T substituted for a U) can be used interchangeably to code for each specific amino acid:

TABLE 1

| Amino Acids | Codons |
|---|---|
| Phenylalanine (Phe or F) | UUU, UUC, UUA or UUG |
| Leucine (Leu or L) | CUU, CUC, CUA or CUG |
| Isoleucine (Ile or I) | AUU, AUC or AUA |
| Methionine (Met or M) | AUG |
| Valine (Val or V) | GUU, GUC, GUA, GUG |
| Serine (Ser or S) | AGU or AGC |
| Proline (Pro or P) | CCU, CCC, CCA, CCG |
| Threonine (Thr or T) | ACU, ACC, ACA, ACG |
| Alanine (Ala or A) | GCU, GCG, GCA, GCC |
| Tryptophan (Trp or W) | UGG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | AGA or AGG |
| Glycine (Gly or G) | GGU or GGC or GGA or GGG |
| Termination codon | UAA, UAG or UGA |

An embodiment of the invention relates to genetic markers for economically valuable traits in animals. The markers represent polymorphic variation or alleles that are associated significantly with growth and/or meat quality and thus provide a method of screening animals to determine those more likely to produce desired traits. As used herein the term “marker” shall include a polymorphic variant capable of detection which may be linked to a quantitative trait loci and thus useful for assaying for the particular trait in the QTL.

Thus, the invention relates to genetic markers and methods of identifying those markers in an animal of a particular breed, strain, population, or group, whereby the animal is more likely to yield favorable boar taint traits.

Genetic markers associated with skatole metabolism, androstenone metabolism and concomitant boar taint are provided herein. The markers are located within the major effect genes of 3αHSD, 3βHSD, CYP17A1, CYP2A, CYP2E1, CYTB5, BAC-CT and/or SULT1A1. The markers can be identified through linkage disequilibrium or association assessment methods described herein or known to those of skill in the art, and provide scores or results indicative of linkage disequilibrium with a chromosomal region/DNA segment or gene or of association with skatole metabolism, androstenone metabolism and concomitant boar taint when tested by such assessment methods. The genetic markers may be associated with skatole metabolism, androstenone metabolism and concomitant boar taint as individual markers and/or in combinations, such as haplotypes, that are in biologically useful association with skatole metabolism, androstenone metabolism and concomitant boar taint.

A genetic marker is a DNA segment with an identifiable location in a chromosome. Genetic markers may be used in a variety of genetic studies such as, for example, locating the chromosomal position or locus of a DNA sequence of interest, and determining if a subject is predisposed to or has a particular boar taint trait:

Because DNA sequences that are relatively close together on a chromosome tend to be inherited together, tracking of a genetic marker through generations in a population and comparing its inheritance to the inheritance of another DNA sequence of interest can provide information useful in determining the relative position of the DNA sequence of interest on a chromosome. Genetic markers particularly useful in such genetic studies are polymorphic. Such markers also may have an adequate level of heterozygosity to allow a reasonable probability that a randomly selected animal will be heterozygous.

The occurrence of variant forms of a particular DNA sequence, e.g., a gene, is referred to as polymorphism. A region of a DNA segment in which variation occurs may be referred to as a polymorphic region or site. A polymorphic region can be a single nucleotide (single nucleotide polymorphism or SNP), the identity of which differs, e.g., in different alleles, or can be two or more nucleotides in length. For example, variant forms of a DNA sequence may differ by an insertion or deletion of one or more nucleotides, insertion of a sequence that was duplicated, inversion of a sequence or conversion of a single nucleotide to a different nucleotide. Each animal can carry two different forms of the specific sequence or two identical forms of the sequence.

Differences between polymorphic forms of a specific DNA sequence may be detected in a variety of ways. For example, if the polymorphism is such that it creates or deletes a restriction enzyme site, such differences may be traced by using restriction enzymes that recognize specific DNA sequences. Restriction enzymes cut (digest) DNA at sites in their specific recognized sequence, resulting in a collection of fragments of the DNA. When a change exists in a DNA sequence that alters a sequence recognized by a restriction enzyme to one not recognized, the fragments of DNA produced by restriction enzyme digestion of the region will be of different sizes. The various possible fragment sizes from a given region therefore depend on the precise sequence of DNA in the region. Variation in the fragments produced is termed “restriction fragment length polymorphism” (RFLP). The different sized-fragments reflecting variant DNA sequences can be visualized by separating the digested DNA according to its size on an agarose gel and visualizing the individual fragments by annealing to a labeled, e.g., radioactively or otherwise labeled, DNA “probe”.

PCR-RFLP, broadly speaking, is a technique that involves obtaining the DNA to be studied, amplifying the DNA, digesting the DNA with restriction endonucleases, separating the resulting fragments, and detecting the fragments of various genes. The use of PCR-RFLPs is the preferred method of detecting the polymorphisms, disclosed herein. However, since the use of RFLP analysis depends ultimately on polymorphisms and DNA restriction sites along the nucleic acid molecule, other methods of detecting the polymorphism can also be used and are contemplated in this invention. Such methods include ones that analyze the polymorphic gene product and detect polymorphisms by detecting the resulting differences in the gene product.

SNP markers may also be used in fine mapping and association analysis, as well as linkage analysis (see, e.g., Kruglyak (1997) Nature Genetics 17:21-24). Although a SNP may have limited information content, combinations of SNPs (which individually occur about every 100-300 bases) may yield informative haplotypes. SNP databases are available. Assay systems for determining SNPs include synthetic nucleotide arrays to which labeled, amplified DNA is hybridized (see, e.g., Lipshutz et al. (1999) Nature Genet. 21:2-24); single base primer extension methods (Pastinen et al. (1997) Genome Res. 7:606-614), mass spectroscopy on tagged beads, and solution assays in which allele-specific oligonucleotides are cleaved or joined at the position of the SNP allele, resulting in activation of a fluorescent reporter system (see, e.g., Landegren et al. (1998) Genome Res. 8:769-776).

### Genetic Association

When two loci are extremely close together, recombination between them is very rare, and the rate at which the two neighboring loci recombine can be so slow as to be unobservable except over many generations. The resulting allelic association is generally referred to as linkage disequilibrium. Linkage disequilibrium can be defined as specific alleles at two or more loci that are observed together on a chromosome more often than expected from their frequencies in the population. As a consequence of linkage disequilibrium, the frequency of all other alleles present in a haplotype carrying a trait-causing allele will also be increased (just as the trait-causing allele is increased in an affected, or trait-positive, population) compared to the frequency in a trait-negative or random control population. Therefore, association between the trait and any allele in linkage disequilibrium with the trait-causing allele will suffice to suggest the presence of a trait-related DNA segment in that particular region of a chromosome. On this basis, association studies are used in methods of locating and discovering methods, as disclosed herein, of identifying an allele that is associated with meat quality and growth traits in animals.

A marker locus must be tightly linked to the trait locus in order for linkage disequilibrium to exist between the loci. In particular, loci must be very close in order to have appreciable linkage disequilibrium that may be useful for association studies. Association studies rely on the retention of adjacent DNA variants over many generations in historic ancestries, and thus, trait-associated regions are theoretically small in outbred random mating populations.

The power of genetic association analysis to detect genetic contributions to traits can be much greater than that of linkage studies. Linkage analysis can be limited by a lack of power to exclude regions or to detect loci with modest effects. Association tests can be capable of detecting loci with smaller effects (Risch and Merikangas (1996) Science 273:1516-1517), which may not be detectable by linkage analysis.

The aim of association studies when used to discover genetic variation in genes associated with phenotypic traits is to identify particular genetic variants that correlate with the phenotype at the population level. Association at the population level may be used in the process of identifying a gene or DNA segment because it provides an indication that a particular marker is either a functional variant underlying the trait (i.e., a polymorphism that is directly involved in causing a particular trait) or is extremely close to the trait gene on a chromosome. When a marker analyzed for association with a phenotypic trait is a functional variant, association is the result of the direct effect of the genotype on the phenotypic outcome. When a marker being analyzed for association is an anonymous marker, the occurrence of association is the result of linkage disequilibrium between the marker and a functional variant.

There are a number of methods typically used in assessing genetic association as an indication of linkage disequilibrium, including case-control study of unrelated animals and methods using family-based controls. Although the case-control design is relatively simple, it is the most prone to identifying DNA variants that prove to be spuriously associated (i.e., association without linkage) with the trait. Spurious association can be due to the structure of the population studied rather than to linkage disequilibrium. Linkage analysis of such spuriously associated allelic variants, however, would not detect evidence of significant linkage because there would be no familial segregation of the variants. Therefore, putative association between a marker allele and a skatole and/or androstenone metabolism, androstenone metabolism and concomitant boar taint trait identified in a case-control study should be tested for evidence of linkage between the marker and the trait before a conclusion of probable linkage disequilibrium is made. Association tests that avoid some of the problems of the standard case-control study utilize family-based controls in which parental alleles or haplotypes not transmitted to affected offspring are used as controls.

In contrast to genetic linkage, which is a property of loci, genetic association is a property of alleles. Association analysis involves a determination of a correlation between a single, specific allele and a trait across a population, not only within individual groups. Thus, a particular allele found through an association study to be in linkage disequilibrium with a skatole and/or androstenone metabolism, androstenone metabolism and thus boar taint associated-allele can form the basis of a method of determining a predisposition to or the occurrence of the trait in any animal. Such methods would not involve a determination of phase of an allele and thus would not be limited in terms of the animals that may be screened in the method.

Methods for Identifying Genetic Markers Associated with Skatole Metabolism, Androstenone Metabolism and Concomitant Boar Taint

Also provided herein are methods of determining a set of genetic markers, which may be used to identify and select animals, based upon their skatole metabolism, androstenone metabolism and concomitant boar taint traits. The methods include a step of testing a polymorphic marker within the major effect genes identified herein. The testing may involve genotyping DNA from animals, and possibly be used as a genetic marker for the same in a given group, population or species, with respect to the polymorphic marker and analyzing the genotyping data for association with skatole metabolism, androstenone metabolism and concomitant boar taint using methods described herein and/or known to those of skill in the art.

Oligonucleotides were used in the PCR amplification of genomic DNA for sequences prior to design of specific oligonucleotides for single-nucleotide polymorphism (SNP) detection and genotyping. PCR conditions are exemplified in the Examples section. According to the invention SNPs were identified in the genes as indicated in table 2. The table also indicates some of the associations identifed to date, other associations for the markers are exemplified in the Examples which follow and will be expected based upon larger and different samples.

TABLE 2

| | | | | | | Location |
|---|---|---|---|---|---|---|
| | | | | | | (bp relative |
| | | | | | | to start |
| | | | SNP | Examples where | | codon) and |
| Gene | | SNP | Allele1/ | associations were | | GenBank |
| Code | Gene Name | Code | allele2 | detected | | entry detail |
| SULT1A1 | sulfotransferase | 140CP | C/T | Skatole | LW_Duroc | 120 |
| | 1A1 | | | Androstenone | Duroc | |
| SULT1A1 | sulfotransferase | 141CP | A/G | Skatole | Duroc | 334 |
| | 1A1 | | | | Hampshire | |
| | | | | | Landrace | |
| CYP2E1 | cytochrome P450 | 152CP | C/T | Skatole | Duroc | 1422 bp |
| | 2E1 | | | | Pietrain | |
| | | | | Androstenone | Pietrain | |
| CYP2E1 | cytochrome P450 | 153CP | A/G | Skatole | Duroc | 1423 |
| | 2E1 | | | | LW_Duroc | |
| CYTB5 | cytochrome B5 | 156CP | G/T | Skatole | Duroc | −8 |
| | | | | | Landrace | |
| | | | | Androstenone | LW_Duroc | |
| | | | | | Yorkshire | |
| 3αHSD | 3 alpha | 157CP | C/T | Skatole | Duroc | 144 |
| | hydroxysteroid | | | | Pietrain | |
| | dehydrogenase | | | | | |
| CYP2E1 | cytochrome P450 | 158CP | G/T | Skatole | Duroc | 1502 |
| | 2E1 | | | | | |
| CYTB5 | cytochrome B5 | 161CP | A/G | Skatole | Duroc | 1500 |
| | | | | | Hampshire | |
| | | | | | Landrace | |
| | | | | | Sireline | |
| | | | | Androstenone | Duroc | |
| | | | | | Landrace | |
| | | | | | Sireline | |
| SULT1Al | sulfotransferase | 162CP | C/T | Skatole | Duroc | −12 |
| | 1A1 | | | | Hampshire | |
| | | | | Androstenone | Duroc | |
| | | | | | Hampshire | |
| | | | | | Sireline | |
| SULT1A1 | sulfotransferase | 171CP | A/G | Skatole | Duroc | Intron 1 |
| | 1A1 | | | | Hampshire | |
| | | | | | Landrace | |
| | | | | | Yorkshire | |
| | | | | Androstenone | Hampshire | |
| CYP17A1 | cytochrome P450 | 173CP | A/G | Skatole | Duroc | Intron 4 |
| | 17A1 | | | | Landrace | |
| | | | | | Pietrain | |
| | | | | | Sireline | |
| | | | | | Yorkshire | |
| | | | | Androstenone | Pietrain | |
| | | | | | Sireline | |
| CYP2E1 | cytochrome P450 | 193CP | C/T | Skatole | Duroc | at position |
| | 2E1 | | | Androstenone | Yorkshire | 2412 |
| | | | | | | in Genbank |
| | | | | | | accession |
| | | | | | | number |
| | | | | | | AJ697882. |
| 3βHSD | 3 beta | 221CP | C/T | Skatole | Large White | −15 |
| | hydroxysteroid | | | | Pietrain | |
| | dehydrogenase | | | | Yorkshire | |
| | | | | Androstenone | Large White | |
| | | | | | Pietrain | |
| | | | | | Sireline | |
| | | | | | Yorkshire | |
| 3βHSD | 3 beta | 222CP | A/G | Skatole | Duroc | 830 |
| | hydroxysteroid | | | | Large White | |
| | dehydrogenase | | | | Yorkshire | |
| | | | | Androstenone | Duroc | |
| | | | | | Landrace | |
| | | | | | Pietrain | |
| BAC-CT | BAC end | 223CP | C/T | Skatole | LW_Duroc | 166 |
| | sequence | | | Androstenone | Duroc | |
| | CT171681 | | | | Pietrain | |
| BAC-CT | BAC end | 224CP | A/G | Skatole | LW_Duroc | 523 |
| | sequence | | | Androstenone | Duroc | |
| | CT171681 | | | | Pietrain | |
| BAC-CT | BAC end | 225CP | A/C | Skatole | LW_Duroc | 707 |
| | sequence | | | Androstenone | Duroc | |
| | CT171681 | | | | Pietrain | |
| BAC-CT | BAC end | 226CP | A/G | Skatole | LW_Duroc | 745 |
| | sequence | | | Androstenone | Duroc | |
| | CT171681 | | | | Pietrain | |
| CYP2A | cytochrome P450 | 238CP | A/G | Skatole | Duroc | −1596 |
| | 2A6 | | | | LW_Duroc | |
| | | | | | Pietrain | |
| | | | | Androstenone | Hampshire | |
| | | | | | LW_Duroc | |
| CYP2A | cytochrome P450 | 239CP | A/G | Skatole | Landrace | −1019 |
| | 2A6 | | | | Large White | |
| | | | | | Sireline | |
| | | | | | Yorkshire | |
| | | | | Androstenone | Duroc | |
| | | | | | Landrace | |
| CYP2A | cytochrome P450 | 240CP | C/T | Androstenone | Sireline | −968 |
| | 2A6 | | | | | |

Any method of identifying the presence or absence of these polymorphisms may be used, including for example single-strand conformation polymorphism (SSCP) analysis, base excision sequence scanning (BESS), RFLP analysis, heteroduplex analysis, denaturing gradient gel electrophoresis, and temperature gradient electrophoresis, allelic PCR, ligase chain reaction, direct sequencing, primer extension, Pyrosequencing, nucleic acid hybridization, micro-array-type detection of a major effect gene or allele, or other linked sequences of the same. Also within the scope of the invention includes assaying for protein conformational or sequences changes, which occur in the presence of this polymorphism. The polymorphism may or may not be the causative mutation but will be indicative of the presence of this change and one may assay for the genetic or protein basis for the phenotypic difference. Based upon detection of these markers, allele frequencies may be calculated for a given population to determine differences in allele frequencies between groups of animals, i.e. the use of quantitative genotyping. This will provide for the ability to select specific populations for associated traits.

Table 3 is a list of markers and primers which were used according of the invention.

TABLE 3

| SNP | Gene | | Annealing | Primer | |
|---|---|---|---|---|---|
| code | Code | Gene Name | Temperature | Name | Primer Sequence (5′-3′) |
| 140CP | SULT1A1 | sulfotransferase 1A1 | 58 | 140CP-F | GTACTTTGCAGAGGCACTGG |
| | | | | | (SEQ ID NO: 1) |
| | | | | 140CP-R | GATTTGGGATAGGTGCTGATC |
| | | | | | (SEQ ID NO: 2) |
| 141CP | SULT1A1 | sulfotransferase 1A1 | 58 | 141CP-F | GTTTTGAGCTGCTGAAAGATACAC |
| | | | | | (SEQ ID NO: 3) |
| | | | | 141CP-R | CTGGTCCAGCAGAGTCTGG |
| | | | | | (SEQ ID NO: 4) |
| 152CP | CYP2E1 | cytochrome P450 2E1 | Touch-down | 152/3CP-F | TGACCCCAAGGATATCGAC |
| | | | | | (SEQ ID NO: 5) |
| | | | | 152/3CP-R | GCACATCTCCCTCACACTTGT |
| | | | | | (SEQ ID NO: 6) |
| 153CP | CYP2E1 | cytochrome P450 2E1 | Touch-down | 152/3CP-F | TGACCCCAAGGATATCGAC |
| | | | | | (SEQ ID NO: 7) |
| | | | | 152/3CP-R | GCACATCTCCCTCACACTTGT |
| | | | | | (SEQ ID NO: 8) |
| 156CP | CYTB5 | cytochrome B5 | 58 | 156CP-F | GACTCCCACTCTGTTCCGC |
| | | | | | (SEQ ID NO: 9) |
| | | | | 156CP-R | CCAGGGTGTAATACTTCACGG |
| | | | | | (SEQ ID NO: 10) |
| 157CP | 3αHSD | 3 alpha hydroxysteroid | Touch-down | 157CP-F | CCCAAGAGTGAAGCTCTGGA |
| | | dehydrogenase | | | (SEQ ID NO: 11) |
| | | | | 157CP-R | CTCTCTTCACGGTGCCATCT |
| | | | | | (SEQ ID NO: 12) |
| 158CP | CYP2E1 | cytochrome P450 2E1 | 58 | 158CP-F | CAAGTGTGAGGGAGATGTGC |
| | | | | | (SEQ ID NO: 13) |
| | | | | 158CP-R | TTGATTTCCTATGGAGCCC |
| | | | | | (SEQ ID NO: 14) |
| 161CP | CYB5 | cytochrome B5 | 58 | 161CP-F | TGAGCCATGGTGTTCTAGAGA |
| | | | | | (SEQ ID NO: 15) |
| | | | | 161CP-R | CAGGCAGAGGGTGATATACGT |
| | | | | | (SEQ ID NO: 16) |
| 162CP | SULT1A1 | sulfotransferase 1A1 | 58 | 162CP-F | ACTGTTGGGATGTTGTACAGG |
| | | | | | (SEQ ID NO: 17) |
| | | | | 162CP-R | AGTACTTGATGAGAGGGACCC |
| | | | | | (SEQ ID NO: 18) |
| 171CP | SULT1A1 | sulfotransferase 1A1 | 58 | 171CP-F | AAAAGCTTGGTCAGAGAAAGC |
| | | | | | (SEQ ID NO: 19) |
| | | | | 171CP-R | AGTTTTGTGGCAGCTCTCC |
| | | | | | (SEQ ID NO: 20) |
| 173CP | CYP17A1 | cytochrome P450 17A1 | 56 | 173CP-F | CGGGAAATCCTTGAAAACC |
| | | | | | (SEQ ID NO: 21) |
| | | | | 173CP-R | AGTGTCCAAAATGAACCCAA |
| | | | | | (SEQ ID NO: 22) |
| 193CP | CYP2E1 | cytochrome P450 2E1 | 56 | 193CP-F | TTTGGTAGTAATCAGAGATGAACTT |
| | | | | | (SEQ ID NO: 23) |
| | | | | 193CP-R | TGAATTTCACTCCACTTTGG |
| | | | | | (SEQ ID NO: 24) |
| 221CP | 3βHSD | 3 alpha hydroxysteroid | 58 | 221CP-F | AGTGTTTTCTGGTTCCTGGC |
| | | dehydrogenase | | | (SEQ ID NO: 25) |
| | | | | 221CP-R | CTCTGACCCAGAAACCCTC |
| | | | | | (SEQ ID NO: 26) |
| 222CP | 3βHSD | 3 alpha hydroxysteroid | 58 | 222CP-F | ACGACACACCTCCCCAAAG |
| | | dehydrogenase | | | (SEQ ID NO: 27) |
| | | | | 222CP-R | GCCAGCCAGTACCTCAGAGA |
| | | | | | (SEQ ID NO: 28) |
| 223CP | BAC-CT | BAC end sequence | 58 | 223CP-F | TCAGGTTGCTGCTATGGTG |
| | | CT171681 | | | (SEQ ID NO: 29) |
| | | | | 223CP-R | AAGTGGCATCTTCCTCTGAA |
| | | | | | (SEQ ID NO: 30) |
| 224CP | BAC-CT | BAC end sequence | 58 | 224CP-F | CTCTTAGGTCTCCCCCTCG |
| | | CT171681 | | | (SEQ ID NO: 31) |
| | | | | 224CP-R | AACTTAGGGCTCAGACAGGC |
| | | | | | (SEQ ID NO: 32) |
| 225CP | BAC-CT | BAC end sequence | 58 | 225/6CP-F | CCTTTTAACCTGTTTCACCCT |
| | | CT171681 | | | (SEQ ID NO: 33) |
| | | | | 225/6CP-R | GGCAGGTAGGCACAGAGAC |
| | | | | | (SEQ ID NO: 34) |
| 226CP | BAC-CT | BAC end sequence | 58 | 225/6CP-F | CCTTTTAACCTGTTTCACCCT |
| | | CT171681 | | | (SEQ ID NO: 35) |
| | | | | 225/6CP-R | GGCAGGTAGGCACAGAGAC |
| | | | | | (SEQ ID NO: 36) |
| 238CP | CYP2A | cytochrome P450 2A6 | 58 | 238CP-F | ACTGCTGTGGTCCCTGTGT |
| | | | | | (SEQ ID NO: 37) |
| | | | | 238CP-R | TTCTTCCTCCAGTGATGGG |
| | | | | | (SEQ ID NO: 38) |
| 239CP | CYP2A | cytochrome P450 2A6 | Touch-down | 239CP-F | GTCCTCAGCACACCCACAC |
| | | | | | (SEQ ID NO: 39) |
| | | | | 239CP-R | CAGGTCCTTAGGGAAGCCT |
| | | | | | (SEQ ID NO: 40) |
| 240CP | CYP2A | cytochrome P450 2A6 | Touch-down | 239CP-F | GTCCTCAGCACACCCACAC |
| | | | | | (SEQ ID NO: 41) |
| | | | | 239CP-R | CAGGTCCTTAGGGAAGCCT |
| | | | | | (SEQ ID NO: 42) |

In a preferred embodiment, the sequences containing the SNPs of interest can be amplified by PCR using the following protocol: 1 μl of the genomic DNA was used as the template for polymerase chain reaction (PCR). The PCR mixtures (6 μl ) containing 1×PCR buffer (100 mM Tris-HCl, pH 8.8; 500 mM KCl; 1% Triton® X-100), 2.5 mM Mg²⁺ (with the exception of marker 156 CP for which we used 4 mM Mg²⁺), 0.2 mM dNTP, 0.4 mM gene-specific primers and 2.5 U of Dynazyme II Taq polymerase (Finnzymes, Espoo, Finland).

The PCR primers for each marker are indicated in Table 3 and two different PCR profiles were used.

The standard PCR profile used was: 5 mM at 94° C., followed by 38 cycles of 45 sec at 94° C., 45 sec at the annealing temperature, 45 sec at 72° C., and final extension of 7 min at 72° C.

The Touchdown PCR profile used was: 5 min at 94° C., followed by 12 cycles of 45 sec at 94° C., 45 sec at 65° C. (decreasing by 1° C. per cycle), 45 sec at 72° C., followed by 26 cycles of 45 sec at 94° C., 45 sec at 52° C., 45 sec at 72° C. and final extension of 7 mM at 72° C.

The SNP of interest contained in the amplicon can then be analysed by one of the genotyping methods described below.

In general, the polymorphisms used as genetic markers of the present invention find use in any method known in the art to demonstrate a statistically significant correlation between a genotype and a phenotype.

The invention therefore, comprises in one embodiment, a method of identifying an allele that is associated with boar taint. The invention also comprises methods of determining a genetic region or marker which may be used to identify and select animals based upon their propensity for boar taint. Yet another embodiment provides a method of identifying an animal for its propensity for boar taint.

Also provided herein are methods of detecting an association between a genotype and a phenotype, which may comprise the steps of a) genotyping at least one candidate gene-related marker in a trait positive population according to a genotyping method of the invention; b) genotyping the candidate gene-related marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination. Preferably, the candidate gene-related marker is present in one or more of the genes listed in table 1. Each of said genotyping of steps a) and b) is performed separately on biological samples derived from each pig in said population or a subsample thereof. Preferably, the phenotype is a trait involving androstenone and/or skatole metabolism or boar taint in a pig.

The invention described herein contemplates alternative approaches that can be employed to perform association studies: genome-wide association studies, candidate region association studies and candidate gene association studies. In a preferred embodiment, the markers of the present invention are used to perform candidate gene association studies. Further, the markers of the present invention may be incorporated in any map of genetic markers of the pig genome in order to perform genome-wide association studies. Methods to generate a high-density map of markers are well known to those of skill in the art. The markers of the present invention may further be incorporated in any map of a specific candidate region of the genome (a specific chromosome or a specific chromosomal segment for example).

Association studies are extremely valuable as they permit the analysis of sporadic or multifactor traits. Moreover, association studies represent a powerful method for fine-scale mapping, enabling much finer mapping of trait causing alleles than linkage studies. Once a chromosome segment of interest has been identified, the presence of a candidate gene such as a candidate gene of the present invention, in the region of interest can provide a shortcut to the identification of the trait causing allele. Polymorphisms used as genetic markers of the present invention can be used to demonstrate that a candidate gene is associated with a trait. Such uses are specifically contemplated in the present invention and claims.

### Association Analysis

The general strategy to perform association studies using markers derived from a region carrying a candidate gene is to scan two groups of animals (case-control populations) in order to measure and statistically compare the allele frequencies of the markers of the present invention in both groups.

If a statistically significant association with a trait is identified for at least one or more of the analyzed markers, one can assume that: either the associated allele is directly responsible for causing the trait (the associated allele is the trait causing allele), or more likely the associated allele is in linkage disequilibrium with the trait causing allele. The specific characteristics of the associated allele with respect to the candidate gene function usually gives further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker.

Association studies are usually run in two successive steps. In a first phase, the frequencies of a reduced number of markers from the candidate gene are determined in the trait positive and trait negative populations. In a second phase of the analysis, the position of the genetic loci responsible for the given trait is further refined using a higher density of markers from the relevant region. However, if the candidate gene under study is relatively small in length, a single phase may be sufficient to establish significant associations.

### Testing for Association

Methods for determining the statistical significance of a correlation between a phenotype and a genotype, in this case an allele at a marker or a haplotype made up of such alleles, may be determined by any statistical test known in the art and is with any accepted threshold of statistical significance being required. The application of particular methods and thresholds of significance are well with in the skill of the ordinary practitioner of the art.

Testing for association is performed in one way by determining the frequency of a marker allele in case and control populations and comparing these frequencies with a statistical test to determine if there is a statistically significant difference in frequency which would indicate a correlation between the trait and the marker allele under study. Similarly, a haplotype analysis is performed by estimating the frequencies of all possible haplotypes for a given set of markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used and many exist. Preferably the statistical test employed is a chi-square test with one degree of freedom. A P-value is calculated (the P-value is the probability that a statistic as large or larger than the observed one would occur by chance). Other methods involve linear models and analysis of variance techniques.

### Genetic Assays

The following is a general overview of techniques which can be used to assay for the polymorphisms of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

### Isolation and Amplification of Nucleic Acid

Samples of genomic DNA are isolated from any convenient source including saliva, buccal cells, hair roots, blood, amniotic fluid, interstitial fluid, peritoneal fluid, chorionic villus, and any other suitable cell or tissue sample with intact interphase nuclei or metaphase cells. The cells can be obtained from solid tissue as well as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the major effect gene of the invention as described in Sambrook et al., supra.

RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

### PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl ) in a buffer of 50 mM Tris-HCl pH 8.3), 50 mM KCl 1.5 mM MgCl₂, 0.5% Tween 20, 0.5% NP40 supplemented with 100 μg/ml of proteinase K. After incubating at 56° C. for 2 hr. the cells are heated to 95° C. for 10 min. to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 pl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6 M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn HoffMan-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

### Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

### Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wild type allele.

### Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (TM). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

### Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

### Single-Strand Conformation Polymorphism Analysis

Target sequences or alleles at an particular locus can be differentiated using single-strand conformation polymorphism analysis, which identifies base differences by alteration in electrophoretic migration of single stranded PCR products, as described in Orita et al., Proc. Nat. Acad. Sci. 85:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

### Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

### Non-gel Systems

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e. there is a mismatch of some form; the cleavage of the dye does not take place. Thus only if the nucleotide sequence of the oligonucleotide probe is completely complementary to the template molecule to which it is bound will quenching be removed. A reaction mix can contain two different probe sequences each designed against different alleles that might be present thus allowing the detection of both alleles in one reaction.

Yet another technique includes an Invader Assay which includes isothermic amplification that relies on a catalytic release of fluorescence. See Third Wave Technology at www.twt.com.

### Non-PCR Based DNA Diagnostics

The identification of a DNA sequence linked to an allele sequence can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with 32P or 35S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a porcine chromosome where one of the major effect genes resides, and thus defining a genetic marker linked to one of the major effect genes, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Indeed in some situations it may be preferable to use combinations of markers giving specific. haplotypes. Additional enzymes, constructed probes and primers can be determined through routine experimentation, combined with the teachings provided and incorporated herein.

According to one embodiment of the invention, polymorphisms in major effect genes have been identified which have an association with skatole metabolism, androstenone metabolism or boar taint. The presence or absence of the markers, in one embodiment may be assayed by PCR RFLP analysis using if needed, restriction endonucleases, and amplification primers which may be designed using analogous human, pig or other of the sequences due to the high homology in the region surrounding the polymorphisms, or may be designed using known sequences (for example, human) as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, Fourth Edition, John Wiley and Sons 1999. The following is a brief description of primer design.

### Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

### Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above (see prediction of Nucleic Acid Structure). If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate animal DNA, genetically type individual animals, and detect genetic differences in animals. In particular, a sample of animal genomic DNA may be evaluated by reference to one or more controls to determine if a polymorphism in one of the sequences is present. Preferably, RFLP analysis is performed with respect to the animal's sequences, and the results are compared with a control. The control is the result of a RFLP analysis of one or both of the sequences of a different animal where the polymorphism of the animal gene is known. Similarly, the genotype of an animal may be determined by obtaining a sample of its genomic DNA, conducting RFLP analysis of the gene in the DNA, and comparing the results with a control. Again, the control is the result of RFLP analysis of one of the sequences of a different animal. The results genetically type the animal by specifying the polymorphism(s) in its gene. Finally, genetic differences among animals can be detected by obtaining samples of the genomic DNA from at least two animals, identifying the presence or absence of a polymorphism in one of the nucleotide sequences, and comparing the results.

These assays are useful for identifying the genetic markers relating to skatole metabolism, androstenone metabolism, or boar taint, as discussed above, for identifying other polymorphisms in the same genes or alleles that may be correlated with other characteristics, and for the general scientific analysis of animal genotypes and phenotypes.

One of skill in the art, once a polymorphism has been identified and a correlation to a particular trait established, will understand that there are many ways to genotype animals for this polymorphism. The design of such alternative tests merely represents optimization of parameters known to those of skill in the art and is intended to be within the scope of this invention as fully described herein.

In accordance with the present invention there may be employed conventional molecular biology, microbiology, and recombinant DNA techniques within the skill of the art. Such techniques are explained fully in the literature. See, e.g., Maniatis, Fritsch & Sambrook, Molecular Cloning: A Laboratory Manual (1982); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription and Translation (B. D. Harries & S. J. Higgins eds. (1984)); Animal Cell Culture (R. I. Freshney, ed. (1986)); Immobilized Cells And Enzymes (IRL Press, (1986)); B. Perbal, A Practical Guide To Molecular Cloning, (1984).

The following examples serves to better illustrate the invention described herein and are not intended to limit the invention in any way. Those skilled in the art will recognize that there are several different parameters which may be altered using routine experimentation and which are intended to be within the scope of this invention.

### Example 1

The following tables include data showing associations between the markers and androstenone and skatole content in fat. Androstenone in back fat was measured using an ELISA method described in Squires, E. J. and K. Lundström 1997. Relationship between cytochrome P450IIE1 in liver and levels of skatole and its metabolites in entire male pigs. J. Anim. Sci. 75:2506-2511. Skatole in back fat was measured using a HPLC method described in Dehnhard, M., Claus, R., Hillenbrand, M. and A Herzog, 1993. High-performance liquid chromatographic method for the determination of 3-methylindole (skatole) and indole in adipose tissue of pigs. J. Chromatogr. 616:205-209.

As can be seen from the tables significant associations exist for one or both of the alleles in one or more populations of different lines of pigs with either skatole or androstenone. Certain of these markers do not show significant associations for these particular populations: however it is expected that with a larger sample size such associations will be evidenced. The detailed results of the single marker analysis were conducted on Log transformed data (Skatole and Androstenone).

The natural logarithm (ln) transformation was used to transform the variables in the following tables prior to the analysis.

| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | 0.569 | 0.172 | | 0.042 | 0.029 | | −0.001 |
| CYP2A | 240CP | BT_240 | ANDRO | Pvalue | 0.259 | | 0.874 | 0.488 | | 0.169 | 0.221 | | 0.828 | 0.899 | | 0.887 |
| CYP2A | 240CP | BT_240 | ANDRO | LSM | 0.17< | 0.45 | <0.63 | −0.36< | 0.046 | >−1 | 0.71< | 0.95 | <1.09 | −0.61< | −0.68 | <−0.55 |
| CYP2A | 240CP | BT_240 | ANDRO | Contrasts | 0.284 | 0.649 | 0.269 | 0.167 | 0.268 | 0.486 | 0.180 | 0.684 | 0.221 | 0.862 | 0.949 | 0.899 |
| CYP2A | 240CP | BT_240 | ANDRO | N | 46. | 50 | .10 | 90. | 24 | .2 | 139. | 55 | .14 | 106. | 59 | .5 |
| CYP2A | 240CP | BT_240 | ANDRO | Freq | 106 | | 0.67 | 116 | | 0.88 | 208 | | 0.80 | 170 | | 0.80 |
| CYP2A | 240CP | BT_240 | SKAT | Effect | −0.067 | | −0.270 | 0.072 | | −0.180 | 0.019 | | 0.140 | −0.110 | | −0.004 |
| CYP2A | 240CP | BT_240 | SKAT | Effect/ | −0.052 | | −0.207 | 0.062 | | −0.151 | 0.019 | | 0.141 | −0.098 | | −0.004 |
| | | | | RMSE | | | | | | | | | | | | |
| CYP2A | 240CP | BT_240 | SKAT | Pvalue | 0.765 | | 0.355 | 0.863 | | 0.716 | 0.891 | | 0.469 | 0.670 | | 0.989 |
| CYP2A | 240CP | BT_240 | SKAT | LSM | 3.9> | 3.56 | <3.76 | 4.6> | 4.49 | <4.74 | 4.53< | 4.69 | >4.57 | 4.14> | 4.02 | >3.91 |
| CYP2A | 240CP | BT_240 | SKAT | Contrasts | 0.214 | 0.659 | 0.785 | 0.706 | 0.773 | 0.689 | 0.316 | 0.664 | 0.891 | 0.537 | 0.640 | 0.670 |
| CYP2A | 240CP | BT_240 | SKAT | N | 46. | 47 | .10 | 76. | 24 | .2 | 134. | 55 | .14 | 104. | 58 | .5 |
| CYP2A | 240CP | BT_240 | SKAT | Freq | 103 | | 0.67 | 102 | | 0.66 | 203 | | 0.80 | 167 | | 0.80 |
| CYP2E1 | 152CP | BT_152 | ANDRO | Effect | 0.320 | | −0.310 | 0.140 | | −0.550 | 0.160 | | −0.130 | 0.150 | | −0.280 |
| CYP2E1 | 152CP | BT_152 | ANDRO | Effect/ | 0.270 | | −0.264 | 0.110 | | −0.431 | 0.164 | | −0.114 | 0.158 | | −0.291 |
| | | | | RMSE | | | | | | | | | | | | |
| CYP2E1 | 152CP | BT_152 | ANDRO | Pvalue | 0.242 | | 0.501 | 0.669 | | 0.171 | 0.316 | | 0.574 | 0.340 | | 0.177 |
| CYP2E1 | 152CP | BT_152 | ANDRO | LSM | 0.31< | 0.32 | <0.96 | −0.19> | −0.6 | <0.089 | 0.76< | 0.81 | <1.12 | −0.58> | −0.71 | <−0.27 |
| CYP2E1 | 152CP | BT_152 | ANDRO | Contrasts | 0.884 | 0.332 | 0.242 | 0.131 | 0.312 | 0.668 | 0.736 | 0.411 | 0.318 | 0.426 | 0.194 | 0.340 |
| CYP2E1 | 152CP | BT_152 | ANDRO | N | 91. | 10 | .5 | 82. | 31 | .4 | 133. | 70 | .10 | 110. | 53 | .10 |
| CYP2E1 | 152CP | BT_152 | ANDRO | Freq | 106 | | 0.91 | 117 | | 0.83 | 213 | | 0.79 | 173 | | 0.79 |
| CYP2E1 | 152CP | BT_152 | SKAT | Effect | 0.670 | | 0.370 | 0.018 | | 0.032 | −0.170 | | 0.280 | −0.045 | | 0.029 |
| CYP2E1 | 152CP | BT_152 | SKAT | Effect/ | 0.542 | | 0.297 | 0.016 | | 0.027 | −0.175 | | 0.286 | −0.039 | | 0.025 |
| | | | | RMSE | | | | | | | | | | | | |
| CYP2E1 | 152CP | BT_152 | SKAT | Pvalue | 0.020 | | 0.449 | 0.958 | | 0.940 | 0.287 | | 0.162 | 0.614 | | 0.908 |
| CYP2E1 | 152CP | BT_152 | SKAT | LSM | 3.57< | 4.6 | <4.9 | 4.58< | 4.63 | >4.62 | 4.57< | 4.67 | >4.23 | 4.09> | 4.07 | >4 |
| CYP2E1 | 152CP | BT_152 | SKAT | Contrasts | 0.013 | 0.665 | 0.020 | 0.653 | 0.985 | 0.958 | 0.460 | 0.175 | 0.287 | 0.934 | 0.853 | 0.814 |
| CYP2E1 | 152CP | BT_152 | SKAT | N | 88. | 10 | .5 | 74. | 26 | .3 | 130. | 68 | .10 | 109. | 51 | .10 |
| CYP2E1 | 152CP | BT_152 | SKAT | Freq | 103 | | 0.90 | 103 | | 0.84 | 208 | | 0.79 | 170 | | 0.79 |
| CYP2E1 | 153CP | BT_153 | ANDRO | Effect | 0.078 | | −0.005 | −0.240 | | −0.170 | 0.160 | | 0.030 | 0.087 | | −0.031 |
| CYP2E1 | 153CP | BT_153 | ANDRO | Effect/ | 0.065 | | −0.004 | −0.183 | | −0.134 | 0.140 | | 0.027 | 0.088 | | −0.032 |
| | | | | RMSE | | | | | | | | | | | | |
| CYP2E1 | 153CP | BT_153 | ANDRO | Pvalue | 0.678 | | 0.984 | 0.338 | | 0.5 | 0.246 | | 0.667 | 0.444 | | 0.840 |
| CYP2E1 | 153CP | BT_153 | ANDRO | LSM | 0.25< | 0.33 | <0.41 | 0.12> | −0.29 | >−0.35 | 0.57< | 0.75 | <0.88 | −0.69< | −0.63 | <−0.51 |
| CYP2E1 | 153CP | BT_153 | ANDRO | Contrasts | 0.837 | 0.744 | 0.678 | 0.405 | 0.797 | 0.333 | 0.491 | 0.433 | 0.246 | 0.799 | 0.473 | 0.444 |
| CYP2E1 | 153CP | BT_153 | ANDRO | N | 14. | 56 | .36 | 8. | 51 | .58 | 21. | 94 | .98 | 27. | 84 | .62 |
| CYP2E1 | 153CP | BT_153 | ANDRO | Freq | 106 | | 0.40 | 117 | | 0.29 | 213 | | 0.32 | 173 | | 0.40 |
| CYP2E1 | 153CP | BT_153 | SKAT | Effect | 0.750 | | 0.013 | 0.092 | | 0.017 | 0.150 | | 0.240 | −0.035 | | −0.042 |
| CYP2E1 | 153CP | BT_153 | SKAT | Effect/ | 0.625 | | 0.011 | 0.078 | | 0.014 | 0.159 | | 0.244 | −0.030 | | −0.036 |
| | | | | RMSE | | | | | | | | | | | | |
| CYP2E1 | 153CP | BT_153 | SKAT | Pvalue | 0.000 | | 0.958 | 0.682 | 0.053 | 0.653 | | 0.127 | 0.727 | 0.797 | | 0.823 |
| CYP2E1 | 153CP | BT_153 | SKAT | LSM | 2.81< | 3.58 | <4.31 | 4.46< | 4.57 | <4.64 | 4.27< | 4.66 | >4.58 | 4.14> | 4.06 | <4.07 |
| CYP2E1 | 153CP | BT_153 | SKAT | Contrasts | 0.042 | 0.005 | 0.000 | 0.810 | 0.756 | 0.882 | 0.0 | 0.582 | 0.188 | 0.769 | 0.972 | 0.797 |
| CYP2E1 | 153CP | BT_153 | SKAT | N | 13. | 55 | .35 | 8. | 44 | .51 | 21. | 92 | .95 | 26. | 83 | .61 |
| CYP2E1 | 153CP | BT_153 | SKAT | Freq | 103 | | 0.39 | 103 | | 0.29 | 208 | | 0.32 | 170 | | 0.40 |
| CYP2E1 | 158CP | BT_158 | ANDRO | Effect | 0.067 | | −0.026 | −0.240 | | −0.170 | 0.160 | | 0.022 | 0.110 | | 0.031 |
| CYP2E1 | 158CP | BT_158 | ANDRO | Effect/ | 0.056 | | −0.022 | −0.183 | | −0.132 | 0.148 | | 0.020 | 0.109 | | 0.032 |
| | | | | RMSE | | | | | | | | | | | | |
| CYP2E1 | 158CP | BT_158 | ANDRO | Pvalue | 0.733 | | 0.918 | 0.335 | | 0.575 | 0.22 | | 0.900 | 0.352 | | 0.84 |
| CYP2E1 | 158CP | BT_158 | ANDRO | LSM | 0.26< | 0.3 | <0.4 | 0.12> | −0.28 | >−0.35 | 0.57< | 0.75 | <0.89 | −0.76< | −0.62 | <−0.55 |
| CYP2E1 | 158CP | BT_158 | ANDRO | Contrasts | 0.913 | 0.722 | 0.733 | 0.410 | 0.795 | 0.335 | 0.487 | 0.380 | 0.221 | 0.631 | 0. [symbol] | 0.352 |
| CYP2E1 | 158CP | BT_158 | ANDRO | N | 13. | 55 | .35 | 8. | 50 | .58 | 21. | 93 | .96 | 26. | 81 | .61 |
| CYP2E1 | 158CP | BT_158 | ANDRO | Freq | 103 | | 0.39 | 116 | | 0.28 | 210 | | 0.32 | 168 | | 0.40 |
| CYP2E1 | 158CP | BT_158 | SKAT | Effect | 0.700 | | −0.050 | 0.092 | | 0.017 | 0.150 | | 0.230 | −0.078 | | −0.072 |
| CYP2E1 | 158CP | BT_158 | SKAT | Effect/ | 0.583 | | −0.041 | 0.078 | | 0.014 | 0.155 | | 0.239 | −0.068 | | −0.063 |
| | | | | RMSE | | | | | | | | | | | | |
| CYP2E1 | 158CP | BT_158 | SKAT | Pvalue | 0.001 | | 0.849 | 0.882 | | 0.953 | 0.200 | | 0.137 | 0.568 | | 0.701 |
| CYP2E1 | 158CP | BT_158 | SKAT | LSM | 2.9< | 3.55 | <4.3 | 4.46< | 4.57 | <4.64 | 4.27< | 4.65 | >4.57 | 4.24> | 4.09 | >4.08 |
| CYP2E1 | 158CP | BT_158 | SKAT | Contrasts | 0.093 | 0.00 | 0.001 | 0.810 | 0.755 | 0.682 | 0.105 | 0. | 0.200 | 0.5 [symbol] | 0. [symbol] | 0. [symbol] |
| CYP2E1 | 158CP | BT_158 | SKAT | N | 12. | 54 | .34 | 8. | 44 | .51 | 21. | 91 | .94 | 25. | 80 | .60 |
| CYP2E1 | 158CP | BT_158 | SKAT | Freq | 100 | | 0.39 | 103 | | 0.29 | 206 | | 0.32 | 165 | | 0.39 |
| CYP2E1 | 193CP | BT_193 | ANDRO | Effect | −0.096 | | 0.030 | 0.240 | | −0.160 | −0.150 | | 0.027 | −0.095 | | −0.014 |
| CYP2E1 | 193CP | BT_193 | ANDRO | Effect/ | −0.079 | | 0.024 | 0.183 | | −0.123 | −0.135 | | 0.024 | −0.096 | | −0.014 |
| | | | | RMSE | | | | | | | | | | | | |
| CYP2E1 | 193CP | BT_193 | ANDRO | Pvalue | 0.650 | | 0.813 | 0.335 | | 0.505 | 0.262 | | 0.880 | 0.415 | | 0.930 |
| CYP2E1 | 193CP | BT_193 | ANDRO | LSM | 0.4> | 0.33 | >0.21 | −0.35< | −0.28 | <0.12 | 0.87> | 0.74 | >0.57 | −0.49> | −0.6 | >−0.68 |
| CYP2E1 | 193CP | BT_193 | ANDRO | Contrasts | 0.803 | 0.757 | 0.650 | 0.759 | 0.425 | 0.335 | 0.449 | 0.511 | 0.262 | 0.624 | 0.720 | 0.415 |
| CYP2E1 | 193CP | BT_193 | ANDRO | N | 35. | 53 | .11 | 58. | 49 | .8 | 97. | 90 | .21 | 60. | 76 | .26 |
| CYP2E1 | 193CP | BT_193 | ANDRO | Freq | 99 | | 0.62 | 113 | | 0.71 | 208 | | 0.68 | 162 | | 0.60 |
| CYP2E1 | 193CP | BT_193 | SKAT | Effect | −0.660 | | −0.007 | −0.100 | | −0.001 | −0.150 | | 0.230 | 0.084 | | −0.005 |
| CYP2E1 | 193CP | BT_193 | SKAT | Effect/ | −0.543 | | −0.006 | −0.087 | | −0.001 | −0.155 | | 0.232 | 0.072 | | −0.005 |
| | | | | RMSE | | | | | | | | | | | | |
| CYP2E1 | 193CP | BT_193 | SKAT | Pvalue | 0.003 | | 0.979 | 0.650 | | 0.996 | 0.200 | | 0.151 | 0.545 | | 0.974 |
| CYP2E1 | 193CP | BT_193 | SKAT | LSM | 4.3> | 3.63 | >2.97 | 4.66> | 4.56 | >4.46 | 4.57< | 4.65 | >4.27 | 4.06< | 4.14 | <4.23 |
| CYP2E1 | 193CP | BT_193 | SKAT | Contrasts | 0.016 | 0.123 | 0.003 | 0.675 | 0.824 | 0.650 | 0.605 | 0.112 | 0.200 | 0.702 | 0.737 | 0.546 |
| CYP2E1 | 193CP | BT_193 | SKAT | N | 34. | 52 | .10 | 49. | 43 | .8 | 94. | 88 | .21 | 59. | 75 | .25 |
| CYP2E1 | 193CP | BT_193 | SKAT | Freq | 96 | | 0.63 | 100 | | 0.71 | 203 | | 0.68 | 159 | | 0.61 |
| CYTB5 | 156CP | BT_156 | ANDRO | Effect | −0.059 | | 0.000 | −0.320 | | 0.910 | −0.900 | | 0.600 | −0.340 | | 0.110 |
| CYTB5 | 156CP | BT_156 | ANDRO | Effect/ | −0.058 | | 0.000 | −0.247 | | 0.708 | −0.816 | | 0.544 | −0.352 | | 0.114 |
| | | | | RMSE | | | | | | | | | | | | |
| CYTB5 | 156CP | BT_156 | ANDRO | Pvalue | 0.826 | | | 0.623 | | 0.295 | 0.006 | | 0.120 | 0.231 | | 0.721 |
| CYTB5 | 156CP | BT_156 | ANDRO | LSM | 0.36> | 0.29 | | −0.31< | 0.28 | >−0.94 | 0.85> | 0.56 | >−0.94 | −0.5> | −0.73 | >−1.19 |
| CYTB5 | 156CP | BT_156 | ANDRO | Contrasts | 0.826 | | | 0.316 | 0.385 | 0.623 | 0.190 | 0.027 | 0.905 | 0.134 | 0.432 | 0.231 |
| CYTB5 | 156CP | BT_156 | ANDRO | N | 69. | 17 | .0 | 111. | 5 | .1 | 182. | 27 | .3 | 103. | 65 | .3 |
| CYTB5 | 156CP | BT_156 | ANDRO | Freq | 106 | | 0.92 | 117 | | 0.97 | 212 | | 0.92 | 171 | | 0.79 |
| CYTB5 | 156CP | BT_156 | SKAT | Effect | −0.890 | | 0.000 | −0.270 | | 0.410 | −0.390 | | 0.100 | 0.091 | | −0.710 |
| CYTB5 | 156CP | BT_156 | SKAT | Effect/ | −0.714 | | 0.000 | −0.230 | | 0.347 | −0.399 | | 0.107 | 0.081 | | −0.639 |
| | | | | RMSE | | | | | | | | | | | | |
| CYTB5 | 156CP | BT_156 | SKAT | Pvalue | 0.00 | | | 0.648 | | 0.607 | 0.172 | | 0.760 | 0.783 | | 0.048 |
| CYTB5 | 156CP | BT_156 | SKAT | LSM | 3.88> | 2.99 | | 4.59< | 4.73 | >4.05 | 4.63> | 4.35 | >3.86 | 4.3> | 3.56 | <4.48 |
| CYTB5 | 156CP | BT_156 | SKAT | Contrasts | 0.00 | | | 0.800 | 0.600 | 0.648 | 0.167 | 0.408 | 0.172 | 0.001 | 0.225 | 0.783 |
| CYTB5 | 156CP | BT_156 | SKAT | N | 86. | 17 | .0 | 97. | 5 | .1 | 178. | 26 | .3 | 100. | 65 | .3 |
| CYTB5 | 156CP | BT_156 | SKAT | Freq | 103 | | 0.92 | 103 | | 0.97 | 207 | | 0.92 | 168 | | 0.79 |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | −0.124 | 0.084 | | 0.061 | −1.143 | | 0.831 |
| CYTB5 | 161CP | BT_161 | ANDRO | Pvalue | 0.064 | | 0.601 | 0.8 | | 0.673 | 0.867 | | 0.016 | 0.024 | | 0.157 |
| CYTB5 | 161CP | BT_161 | ANDRO | LSM | 0.46> | 0.21 | >−0.4 | −0.25> | −0.45 | <−0.32 | 0.78< | 0.95 | <0.97 | −0.57> | −0.86 | >−2.75 |
| CYTB5 | 161CP | BT_161 | ANDRO | Contrasts | 0.373 | 0.231 | 0.064 | 0.514 | 0.832 | 0.899 | 0.626 | 0. [symbol] | 0.867 | 0.319 | 0.060 | 0.024 |
| CYTB5 | 161CP | BT_161 | ANDRO | N | 74. | 22 | .7 | 85. | 24 | .6 | 191. | 12 | .1 | 155. | 11 | .1 |
| CYTB5 | 161CP | BT_161 | ANDRO | Freq | 103 | | 0.83 | 115 | | 0.84 | 204 | | 0.97 | 167 | | 0.96 |
| CYTB5 | 161CP | BT_161 | SKAT | Effect | −0.840 | | −0.450 | 0.340 | | 0.200 | −0.500 | | 0.440 | 0.360 | | −1.230 |
| CYTB5 | 161CP | BT_161 | SKAT | Effect/ | −0.744 | | −0.395 | 0.291 | | 0.169 | −0.511 | | 0.454 | 0.326 | | −1.116 |
| | | | | RMSE | | | | | | | | | | | | |
| CYTB5 | 161CP | BT_161 | SKAT | Pvalue | 0.000 | | 0.183 | 0.210 | | 0.600 | 0.310 | | 0.433 | 0.517 | | 0.058 |
| CYTB5 | 161CP | BT_161 | SKAT | LSM | 4.09> | 2.8 | >2.4 | 4.45< | 4.98 | <5.12 | 4.56> | 4.53 | >3.59 | 4.16> | 3.26 | <4.86 |
| CYTB5 | 161CP | BT_161 | SKAT | Contrasts | 0.000 | 0.425 | 0.000 | 0.070 | 0.807 | 0.210 | 0.850 | 0.355 | 0.310 | 0.012 | 0.1 [symbol] | 0.517 |
| CYTB5 | 161CP | BT_161 | SKAT | N | 72. | 21 | .7 | 76. | 20 | .5 | 188. | 12 | .1 | 152. | 11 | .1 |
| CYTB5 | 161CP | BT_161 | SKAT | Freq | 100 | | 0.83 | 101 | | 0.85 | 201 | | 0.97 | 164 | | 0.96 |
| SULT1A1 | 140CP | BT_140 | ANDRO | Effect | −0.340 | | 0.150 | 0.180 | | −0.029 | 0.130 | | 0.051 | −0.150 | | −0.050 |
| SULT1A1 | 140CP | BT_140 | ANDRO | Effect/ | −0.313 | | 0.140 | 0.143 | | −0.022 | 0.118 | | 0.046 | −0.151 | | −0.051 |
| | | | | RMSE | | | | | | | | | | | | |
| SULT1A1 | 140CP | BT_140 | ANDRO | Pvalue | 0.025 | | 0.4 [symbol] | 0.501 | | 0. [symbol] | 0.1 [symbol] | | 0.759 | 0.161 | | 0.764 |
| SULT1A1 | 140CP | BT_140 | ANDRO | LSM | 0.65> | 0.46 | >−0.025 | −0.34< | −0.19 | <0.024 | 0.67< | 0.85 | <0.83 | −0.4> | −0.59 | >−0.69 |
| SULT1A1 | 140CP | BT_140 | ANDRO | Contrasts | 0.451 | 0.0 [symbol] | 0.02 [symbol] | 0.587 | 0.715 | 0.501 | 0.303 | 0.703 | 0.186 | 0.331 | 0.683 | 0.161 |
| SULT1A1 | 140CP | BT_140 | ANDRO | N | 33. | 46 | .22 | 84. | 27 | .6 | 94. | 69 | .48 | 36. | 67 | .56 |
| SULT1A1 | 140CP | BT_140 | ANDRO | Freq | 101 | | 0.55 | 117 | | 0.63 | 211 | | 0.61 | 159 | | 0.44 |
| SULT1A1 | 140CP | BT_140 | SKAT | Effect | −0.190 | | 0.016 | 0.140 | | −0.110 | 0.130 | | −0.200 | 0.062 | | −0.220 |
| SULT1A1 | 140CP | BT_140 | SKAT | Effect/ | −0.152 | | 0.013 | 0.119 | | −0.091 | 0.131 | | −0.211 | 0.054 | | −0.190 |
| | | | | RMSE | | | | | | | | | | | | |
| SULT1A1 | 140CP | BT_140 | SKAT | Pvalue | 0.27 | | 0.949 | 0.576 | | 0.754 | 0.143 | | 0.164 | 0.618 | | 0.246 |
| SULT1A1 | 140CP | BT_140 | SKAT | LSM | 3.93> | 3.75 | >3.54 | 4.57< | 4.6 | <4.85 | 4.54> | 4.46 | <4.79 | 4.16> | 4 | <4.28 |
| SULT1A1 | 140CP | BT_140 | SKAT | Contrasts | 0.548 | 0.529 | 0.271 | 0.902 | 0.643 | 0.678 | 0. [symbol] | 0.071 | 0. [symbol] | 0.612 | 0.187 | 0. [symbol] |
| SULT1A1 | 140CP | BT_140 | SKAT | N | 33. | 43 | .22 | 71. | 25 | .6 | 91. | 67 | .48 | 36. | 67 | .53 |
| SULT1A1 | 140CP | BT_140 | SKAT | Freq | 98 | | 0.55 | 103 | | 0.82 | 206 | | 0.60 | 156 | | 0.45 |
| SULT1A1 | 141CP | BT_141 | ANDRO | Effect | −0.450 | | −0.750 | −0.470 | | −0.950 | −0.059 | | −0.087 | 0.210 | | 0.140 |
| SULT1A1 | 141CP | BT_141 | ANDRO | Effect/ | −0.375 | | −0.629 | −0.369 | | −0.743 | −0.063 | | −0.078 | 0.214 | | 0.145 |
| | | | | RMSE | | | | | | | | | | | | |
| SULT1A1 | 141CP | BT_141 | ANDRO | Pvalue | 0.457 | | 0.3 [symbol] | 0.304 | | 0.1 [symbol] | 0. [symbol] | | 0.858 | 0.2 [symbol] | | 0. [symbol] |
| SULT1A1 | 141CP | BT_141 | ANDRO | LSM | 126> | 0.065 | <0.37 | 0.67> | −0.76 | <−0.28 | 0.91> | 0.76 | <0.79 | −0.96< | −0.61 | <−0.54 |
| SULT1A1 | 141CP | BT_141 | ANDRO | Contrasts | 0.346 | 0.4 [symbol] | 0.457 | 0.1 [symbol] | 0.340 | 0.304 | 0.861 | 0.920 | 0. [symbol] | 0. [symbol] | 0.720 | 0.241 |
| SULT1A1 | 141CP | BT_141 | ANDRO | N | 1. | 8 | .96 | 2. | 7 | .108 | 2. | 18 | .192 | 8. | 34 | .128 |
| SULT1A1 | 141CP | BT_141 | ANDRO | Freq | 105 | | 0.05 | 117 | | 0.05 | 212 | | 0.05 | 168 | | 0.15 |
| SULT1A1 | 141CP | BT_141 | SKAT | Effect | −0.770 | | 0.430 | −0.810 | | −1.480 | 0.008 | | −0.090 | 0.420 | | 0.160 |
| SULT1A1 | 141CP | BT_141 | SKAT | Effect/ | −0.621 | | 0.345 | −0.708 | | −1.291 | 0.009 | | −0.093 | 0.365 | | 0.142 |
| | | | | RMSE | | | | | | | | | | | | |
| SULT1A1 | 141CP | BT_141 | SKAT | Pvalue | 0.220 | | 0.576 | 0.050 | | 0.016 | 0. [symbol] | | 0.828 | 0.047 | | 0.670 |
| SULT1A1 | 141CP | BT_141 | SKAT | LSM | 5.18> | 4.84 | >3.64 | 6.23> | 3.94 | <4.61 | 4.57> | 4.49 | <4.59 | 3.36< | 3.94 | <4.19 |
| SULT1A1 | 141CP | BT_141 | SKAT | Contrasts | 0.796 | 0.010 | 0.220 | 0.014 | 0.140 | 0.050 | 0. [symbol] | 0.681 | 0.981 | 0. [symbol] | 0.2 [symbol] | 0.047 |
| SULT1A1 | 141CP | BT_141 | SKAT | N | 1. | 8 | .93 | 2. | 7 | .94 | 2. | 18 | .187 | 8. | 34 | .123 |
| SULT1A1 | 141CP | BT_141 | SKAT | Freq | 102 | | 0.05 | 103 | | 0.05 | 207 | | 0.05 | 165 | | 0.15 |
| SULT1A1 | 162CP | BT_162 | ANDRO | Effect | 0.550 | | −0.710 | −0.350 | | −0.330 | −0.023 | | 0.021 | 0.230 | | 0.068 |
| SULT1A1 | 162CP | BT_162 | ANDRO | Effect/ | 0.476 | | −0.612 | −0.281 | | −0.255 | −0.021 | | 0.019 | 0.244 | | 0.071 |
| | | | | RMSE | | | | | | | | | | | | |
| SULT1A1 | 162CP | BT_162 | ANDRO | Pvalue | 0.013 | | 0.020 | 0.021 | | 0.265 | 0.825 | [symbol] | [symbol] | 0.102 | | 0.718 |
| SULT1A1 | 162CP | BT_162 | ANDRO | LSM | 0.28> | 0.12 | <1.39 | 0.27> | −0.41 | > −0.43 | 0.62> | 0.81 | >0.77 | −0.74< | −0.44 | <−0.28 |
| SULT1A1 | 162CP | BT_162 | ANDRO | Contrasts | 0.534 | 0.007 | 0.013 | 0.064 | 0.947 | 0.021 | 0 [symbol] | 0.839 | 0.825 | [symbol] | 0.575 | 0.102 |
| SULT1A1 | 162CP | BT_162 | ANDRO | N | 62. | 32 | .8 | 23. | 24 | .70 | 95. | 74 | .40 | 93. | 59 | .13 |
| SULT1A1 | 162CP | BT_162 | ANDRO | Freq | 102 | | 0.76 | 117 | | 0.30 | 209 | | 0.63 | 165 | | 0.74 |
| SULT1A1 | 162CP | BT_162 | SKAT | Effect | 0.460 | | −0.370 | −0.320 | | −0.750 | 0.046 | | −0.230 | 0.042 | | 0.018 |
| SULT1A1 | 162CP | BT_162 | SKAT | Effect/ | 0.371 | | −0.298 | −0.285 | | −0.665 | 0.048 | | −0.239 | 0.037 | | 0.016 |
| | | | | RMSE | | | | | | | | | | | | |
| SULT1A1 | 162CP | BT_162 | SKAT | Pvalue | 0.052 | | 0.255 | 0.022 | | 0.008 | 0. [symbol] | | 0.118 | 0.803 | | 0.935 |
| SULT1A1 | 162CP | BT_162 | SKAT | LSM | 3.59< | 3.68 | <4.52 | 5.19> | 4.12 | <4.54 | 4.63> | 4.44 | <4.72 | 4.07< | 4.13 | <4.16 |
| SULT1A1 | 162CP | BT_162 | SKAT | Contrasts | 0.744 | | 0.052 | 0.002 | 0.13 | 0.022 | 0.223 | 0.150 | 0. [symbol] | 0.752 | 0.948 | 0.603 |
| SULT1A1 | 162CP | BT_162 | SKAT | N | 60. | 31 | .8 | 23. | 22 | .58 | 94. | 72 | .39 | 90. | 59 | .13 |
| SULT1A1 | 162CP | BT_162 | SKAT | Freq | 99 | | 0.76 | 103 | | 0.33 | 205 | | 0.63 | 162 | | 0.74 |
| SULT1A1 | 171CP | BT_171 | ANDRO | Effect | 0.240 | | −0.330 | −0.350 | | −0.340 | 0.009 | | 0.052 | 0.140 | | 0.076 |
| SULT1A1 | 171CP | BT_171 | ANDRO | Effect/ | 0.200 | | −0.276 | −0.276 | | −0.267 | 0.008 | | 0.046 | 0.139 | | 0.078 |
| | | | | RMSE | | | | | | | | | | | | |
| SULT1A1 | 171CP | BT_171 | ANDRO | Pvalue | 0.147 | | 0.251 | 0.033 | | 0.311 | 0.828 | | 0.756 | 0.191 | | 0.622 |
| SULT1A1 | 171CP | BT_171 | ANDRO | LSM | 0.29> | 0.2 | <0.76 | 0.28> | −0.41 | >−0.42 | 0.75< | 0.81 | >0.77 | −0.74< | −0.53 | <−0.47 |
| SULT1A1 | 171CP | BT_171 | ANDRO | Contrasts | 0.746 | 0.130 | 0.147 | 0.097 | 0.975 | 0.033 | 0.738 | 0.833 | 0.928 | 0.205 | 0.787 | 0.191 |
| SULT1A1 | 171CP | BT_171 | ANDRO | N | 62. | 26 | .17 | 19. | 19 | .78 | 85. | 69 | .54 | 64. | 74 | .34 |
| SULT1A1 | 171CP | BT_171 | ANDRO | Freq | 105 | | 0.71 | 116 | | 0.25 | 208 | | 0.57 | 172 | | 0.59 |
| SULT1A1 | 171CP | BT_171 | SKAT | Effect | 0.540 | | −0.810 | −0.280 | | −0.650 | 0.011 | | −0.180 | −0.180 | | −0.240 |
| SULT1A1 | 171CP | BT_171 | SKAT | Effect/ | 0.443 | | −0.667 | −0.244 | | −0.573 | 0.011 | | −0.183 | −0.158 | | −0.216 |
| | | | | RMSE | | | | | | | | | | | | |
| SULT1A1 | 171CP | BT_171 | SKAT | Pvalue | 0.002 | | 0.007 | 0.054 | | 0.040 | 0.901 | | 0.226 | 0.142 | | 0.174 |
| SULT1A1 | 171CP | BT_171 | SKAT | LSM | 3.63> | 3.36 | <4.71 | 5.09> | 4.16 | <4.54 | 4.62> | 4.45 | <4.64 | 4.35> | 3.92 | <3.99 |
| SULT1A1 | 171CP | BT_171 | SKAT | Contrasts | 0.350 | 0.001 | 0.002 | 0.018 | 0.229 | 0.064 | 0.296 | 0.293 | 0.901 | 0.0.32 | 0.779 | 0.142 |
| SULT1A1 | 171CP | BT_171 | SKAT | N | 80. | 25 | .17 | 19. | 17 | .66 | 83. | 67 | .53 | 61. | 74 | .34 |
| SULT1A1 | 171CP | BT_171 | SKAT | Freq | 102 | | 0.71 | 102 | | 0.27 | 203 | | 0.57 | 169 | | 0.58 |
| BAC-CT | 223CP | BT_223 | ANDRO | Effect | 0.380 | | 0.053 | −0.150 | | 0.160 | 0.180 | | −0.042 | −0.019 | | −0.018 |
| BAC-CT | 223CP | BT_223 | ANDRO | Effect/ | 0.330 | | 0.046 | −0.115 | | 0.127 | 0.161 | | −0.038 | −0.020 | | −0.018 |
| | | | | RMSE | | | | | | | | | | | | |
| BAC-CT | 223CP | BT_223 | ANDRO | Pvalue | 0.038 | | 0.831 | 0.563 | | 0.625 | 0.201 | | 0.833 | 0.905 | | 0.930 |
| BAC-CT | 223CP | BT_223 | ANDRO | LSM | 0.053< | 0.49 | <0.82 | −0.27< | −0.26 | >−0.57 | 0.72< | 0.86 | <1.08 | −0.57> | −0.61 | =−0.61 |
| BAC-CT | 223CP | BT_223 | ANDRO | Contrasts | 0.073 | 0.385 | 0.038 | 0.953 | 0.560 | 0.583 | 0.427 | 0.459 | 0.201 | 0.812 | 0.996 | 0.905 |
| BAC-CT | 223CP | BT_223 | ANDRO | N | 45. | 48 | .13 | 73. | 36 | .7 | 131. | 61 | .18 | 93. | 68 | .10 |
| BAC-CT | 223CP | BT_223 | ANDRO | Freq | 106 | | 0.65 | 116 | | 0.78 | 210 | | 0.77 | 171 | | 0.74 |
| BAC-CT | 223CP | BT_223 | SKAT | Effect | 0.041 | | −0.040 | 0.360 | | −0.220 | −0.042 | | 0.330 | −0.200 | | 0.250 |
| BAC-CT | 223CP | BT_223 | SKAT | Effect/ | 0.032 | | −0.031 | 0.303 | | −0.183 | −0.043 | | 0.339 | −0.172 | | 0.217 |
| | | | | RMSE | | | | | | | | | | | | |
| BAC-CT | 223CP | BT_223 | SKAT | Pvalue | 0.841 | | 0.887 | 0.243 | | 0.652 | 0.731 | | 0.061 | 0.305 | | 0.295 |
| BAC-CT | 223CP | BT_223 | SKAT | LSM | 3.72= | 3.72 | <3.8 | 4.51< | 4.65 | <5.22 | 4.5< | 4.79 | >4.42 | 4.09< | 4.14 | >3.69 |
| BAC-CT | 223CP | BT_223 | SKAT | Contrasts | 0.997 | 0.843 | 0.841 | 0.569 | 0.358 | 0.243 | 0.060 | 0.156 | 0.731 | 0.778 | 0.263 | 0.305 |
| BAC-CT | 223CP | BT_223 | SKAT | N | 45. | 45 | .13 | 62. | 35 | .4 | 127. | 60 | .18 | 91. | 67 | .10 |
| BAC-CT | 223CP | BT_223 | SKAT | Freq | 103 | | 0.66 | 102 | | 0.78 | 205 | | 0.77 | 168 | | 0.74 |
| BAC-CT | 224CP | BT_224 | ANDRO | Effect | −0.380 | | 0.053 | 0.150 | | 0.150 | −0.180 | | −0.049 | 0.019 | | −0.035 |
| BAC-CT | 224CP | BT_224 | ANDRO | Effect/ | −0.330 | | 0.046 | 0.113 | | 0.116 | −0.162 | | −0.044 | 0.020 | | −0.035 |
| | | | | RMSE | | | | | | | | | | | | |
| BAC-CT | 224CP | BT_224 | ANDRO | Pvalue | 0.038 | | 0.831 | 0.668 | | 0.656 | 0.168 | | 0.808 | 0.906 | | 0.863 |
| BAC-CT | 224CP | BT_224 | ANDRO | LSM | 0.82> | 0.49 | >0.053 | −0.57< | −0.27 | >−0.28 | 1.08> | 0.85 | >0.72 | −0.61> | −0.62 | <−0.57 |
| BAC-CT | 224CP | BT_224 | ANDRO | Contrasts | 0.365 | 0.073 | 0.038 | 0.581 | 0.988 | 0.588 | 0.442 | 0.444 | 0.198 | 0.963 | 0.728 | 0.905 |
| BAC-CT | 224CP | BT_224 | ANDRO | N | 13. | 48 | .45 | 7. | 35 | .74 | 18. | 62 | .132 | 10. | 69 | .93 |
| BAC-CT | 224CP | BT_224 | ANDRO | Freq | 106 | | 0.35 | 116 | | 0.21 | 212 | | 0.23 | 172 | | 0.26 |
| BAC-CT | 224CP | BT_224 | SKAT | Effect | −0.041 | | −0.040 | −0.350 | | −0.200 | 0.047 | | 0.300 | 0.200 | | 0.230 |
| [symbol] | [symbol] | [symbol] | [symbol] | [symbol] | [symbol] | [symbol] | [symbol] | [symbol] | [symbol] | [symbol] | [symbol] | [symbol] | 0.000 | 0.137 | | −0.060 |
| 3_alfa-HSD | 157CP | BT_157 | ANDRO | Pvalue | 0.856 | [symbol] | [symbol] | 0.168 | | | [symbol] | | | 0.267 | | 0.746 |
| 3_alfa-HSD | 157CP | BT_157 | ANDRO | LSM | | 0.28 | <0.35 | | 0.21 | >−0.35 | | 0.85 | >0.78 | −0.77< | −0.7 | <−0.5 |
| 3_alfa-HSD | 157CP | BT_157 | ANDRO | Contrasts | | | | | 0.168 | | | 0.741 | | 0.772 | 0.253 | 0.287 |
| 3_alfa-HSD | 157CP | BT_157 | ANDRO | N | 0. | 9 | .97 | 0. | 11 | .105 | 0. [symbol] | 29 | .183 | 20. | 52 | .98 |
| 3_alfa-HSD | 157CP | BT_157 | ANDRO | Freq | 106 | | 0.04 | 116 | | 0.05 | 212 | | 0.07 | 170 | | 0.27 |
| 3_alfa-HSD | 157CP | BT_157 | SKAT | Effect | −0.960 | | 0.000 | −0.590 | | 0.000 | −0.090 | | 0.000 | 0.230 | | 0.091 |
| 3_alfa-HSD | 157CP | BT_157 | SKAT | Effect/ | −0.759 | | 0.000 | −0.512 | | 0.000 | −0.093 | | 0.000 | 0.199 | | 0.079 |
| | | | | RMSE | | | | | | | | | | | | |
| 3_alfa-HSD | 157CP | BT_157 | SKAT | Pvalue | 0.032 | | | 0.127 | | | 0.649 | | | 0.114 | | 0.677 |
| 3_alfa-HSD | 157CP | BT_157 | SKAT | LSM | | 4.6 | >3.65 | | 5.12 | >4.52 | | 4.67 | >4.58 | 3.7< | 4.02 | <4.16 |
| 3_alfa-HSD | 157CP | BT_157 | SKAT | Contrasts | | 0.032 | | | 0.127 | | | 0.649 | | 0.804 | 0.490 | 0.114 |
| 3_alfa-HSD | 157CP | BT_157 | SKAT | N | 0. | 9 | .94 | 0. | 10 | .92 | 0. | 28 | .179 | 19. | 50 | .98 |
| 3_alfa-HSD | 157CP | BT_157 | SKAT | Freq | 103 | | 0.04 | 102 | | 0.05 | 207 | | 0.07 | 167 | | 0.26 |
| 3_beta_HSD | 221CP | BT_221 | ANDRO | Effect | 0.080 | | −0.003 | 0.005 | | 0.140 | 0.008 | | −0.150 | −0.110 | | −0.057 |
| 3_beta_HSD | 221CP | BT_221 | ANDRO | Effect/ | 0.067 | | −0.002 | 0.004 | | 0.109 | 0.007 | | −0.136 | −0.115 | | −0.058 |
| | | | | RMSE | | | | | | | | | | | | |
| 3_beta_HSD | 221CP | BT_221 | ANDRO | Pvalue | 0.646 | | 0.991 | 0.976 | | 0.681 | 0.944 | | 0.339 | 0.356 | | 0.733 |
| 3_beta_HSD | 221CP | BT_221 | ANDRO | LSM | 0.23< | 0.31 | <0.39 | −0.36< | −0.22 | >−0.35 | 0.87> | 0.72 | <0.89 | −0.43> | −0.6 | >−0.66 |
| 3_beta_HSD | 221CP | BT_221 | ANDRO | Contrasts | 0.628 | 0.748 | 0.645 | 0.593 | 0.880 | 0.976 | 0.486 | 0.399 | 0.944 | 0.486 | 0.732 | 0.356 |
| 3_beta_HSD | 221CP | BT_221 | ANDRO | N | 16. | 40 | .48 | 47. | 44 | .24 | 46. | 108 | .52 | 21. | 74 | .73 |
| 3_beta_HSD | 221CP | BT_221 | ANDRO | Freq | 104 | | 0.35 | 115 | | 0.60 | 206 | | 0.49 | 168 | | 0.35 |
| 3_beta_HSD | 221CP | BT_221 | SKAT | Effect | −0.140 | | 0.390 | −0.042 | | 0.068 | −0.088 | | −0.041 | −0.140 | | −0.260 |
| 3_beta_HSD | 221CP | BT_221 | SKAT | Effect/ | −0.113 | | 0.308 | −0.036 | | 0.058 | −0.091 | | −0.042 | −0.124 | | −0.227 |
| | | | | RMSE | | | | | | | | | | | | |
| 3_beta_HSD | 221CP | BT_221 | SKAT | Pvalue | 0.438 | | 0.160 | 0.789 | | 0.786 | 0.368 | | 0.768 | 0.320 | | 0.188 |
| 3_beta_HSD | 221CP | BT_221 | SKAT | LSM | 3.75< | 4 | >3.47 | 4.59< | 4.61 | >4.5 | 4.7> | 4.57 | >4.52 | 4.36> | 3.96 | <4.08 |
| 3_beta_HSD | 221CP | BT_221 | SKAT | Contrasts | 0.516 | 0.058 | 0.438 | 0.923 | 0.733 | 0.789 | 0.464 | 0. [symbol] | 0.368 | 0.159 | 0.638 | 0.320 |
| 3_beta_HSD | 221CP | BT_221 | SKAT | N | 16. | 38 | .47 | 43. | 37 | .21 | 46. | 103 | .52 | 21. | 73 | .71 |
| 3_beta_HSD | 221CP | BT_221 | SKAT | Freq | 101 | | 0.35 | 101 | | 0.61 | 201 | | 0.49 | 165 | | 0.35 |
| 3_beta_HSD | 222CP | BT_222 | ANDRO | Effect | 0.820 | | −0.320 | −0.021 | | −0.160 | 0.082 | | −0.110 | 0.220 | | −0.570 |
| 3_beta_HSD | 222CP | BT_222 | ANDRO | Effect/ | 0.713 | | −0.279 | −0.017 | | −0.124 | 0.073 | | −0.094 | 0.230 | | −0.597 |
| | | | | RMSE | | | | | | | | | | | | |
| 3_beta_HSD | 222CP | BT_222 | ANDRO | Pvalue | 0.006 | | 0.452 | 0.929 | | 0.626 | 0.541 | | 0.605 | 0.099 | | 0.002 |
| 3_beta_HSD | 222CP | BT_222 | ANDRO | LSM | 0.22< | 0.71 | <1.85 | −0.24> | −0.42 | <−0.28 | 0.77> | 0.74 | <0.93 | −0.51> | −0.68 | <−0.072 |
| 3_beta_HSD | 222CP | BT_222 | ANDRO | Contrasts | 0.135 | 0.083 | 0.008 | 0.500 | 0.786 | 0.929 | 0.898 | 0.523 | 0.641 | 0.027 | 0.005 | 0.099 |
| 3_beta_HSD | 222CP | BT_222 | ANDRO | N | 88. | 14 | .4 | 76. | 33 | .8 | 135. | 54 | .20 | 98. | 59 | .15 |
| 3_beta_HSD | 222CP | BT_222 | ANDRO | Freq | 106 | | 0.90 | 117 | | 0.79 | 209 | | 0.78 | 172 | | 0.74 |
| 3_beta_HSD | 222CP | BT_222 | SKAT | Effect | 0.680 | | 0.820 | 0.068 | | −0.010 | 0.083 | | −0.180 | 0.071 | | −0.049 |
| 3_beta_HSD | 222CP | BT_222 | SKAT | Effect/ | 0.580 | | 0.699 | 0.058 | | −0.009 | 0.085 | | −0.185 | 0.061 | | −0.042 |
| | | | | RMSE | | | | | | | | | | | | |
| 3_beta_HSD | 222CP | BT_222 | SKAT | Pvalue | 0.026 | | 0.062 | 0.771 | | 0.978 | 0.478 | | 0.318 | 0.661 | | 0.826 |
| 3_beta_HSD | 222CP | BT_222 | SKAT | LSM | 3.48< | 4.97 | >4.83 | 4.57< | 4.63 | <4.71 | 4.59> | 4.49 | <4.76 | 4.06< | 4.08 | <4.2 |
| 3_beta_HSD | 222CP | BT_222 | SKAT | Contrasts | 0.000 | 0.834 | 0.026 | 0.831 | 0.877 | 0.771 | 0.542 | 0.305 | 0.478 | 0.909 | 0.722 | 0.661 |
| 3_beta_HSD | 222CP | BT_222 | SKAT | N | 85. | 14 | .4 | 70. | 26 | .7 | 131. | 53 | .20 | 95. | 59 | .15 |
| 3_beta_HSD | 222CP | BT_222 | SKAT | Freq | 103 | | 0.89 | 103 | | 0.81 | 204 | | 0.77 | 0.169 | | 0.74 |
| CYP17A1 | 173CP | BT_173 | ANDRO | Effect | −0.150 | | −0.400 | 0.190 | | 0.100 | 0.041 | | 0.120 | 0.120 | | 0.070 |
| CYP17A1 | 173CP | BT_173 | ANDRO | Effect/ | −0.121 | | −0.335 | 0.145 | | 0.079 | 0.037 | | 0.104 | 0.120 | | 0.072 |
| | | | | RMSE | | | | | | | | | | | | |
| CYP17A1 | 173CP | BT_173 | ANDRO | Pvalue | 0.459 | | 0.203 | 0.238 | | 0.873 | 0.760 | | 0.623 | 0.250 | | 0.642 |
| CYP17A1 | 173CP | BT_173 | ANDRO | LSM | 0.65> | 0.1 | <0.36 | −0.52< | −0.23 | <−0.16 | 0.69< | 0.85 | >0.77 | −0.74< | −0.55 | <−0.5 |
| CYP17A1 | 173CP | BT_173 | ANDRO | Contrasts | 0.214 | 0.371 | 0.459 | 0.320 | 0.767 | 0.238 | 0.568 | 0.648 | 0.780 | 0.272 | 0.810 | 0.250 |
| CYP17A1 | 173CP | BT_173 | ANDRO | N | 11. | 24 | .67 | 33. | 50 | .34 | 21. | 83 | .106 | 58. | 75 | 0.38 |
| CYP17A1 | 173CP | BT_173 | ANDRO | Freq | 102 | | 0.23 | 117 | | 0.50 | 210 | | 0.30 | 171 | | 0.56 |
| CYP17A1 | 173CP | BT_173 | SKAT | Effect | −0.630 | | −0.370 | 0.140 | | 0.230 | −0.036 | | 0.029 | 0.280 | | −0.130 |
| CYP17A1 | 173CP | BT_173 | SKAT | Effect/ | −0.519 | | −0.307 | 0.119 | | 0.201 | −0.037 | | 0.030 | 0.245 | | −0.111 |
| | | | | RMSE | | | | | | | | | | | | |
| CYP17A1 | 173CP | BT_173 | SKAT | Pvalue | 0.002 | | 0.243 | 0.364 | | 0.316 | 0.756 | | 0. [symbol] | 0.0 [symbol] | | 0.478 |
| CYP17A1 | 173CP | BT_173 | SKAT | LSM | 4.8> | 3.79 | >3.53 | 4.35< | 4.73 | >4.63 | 4.61> | 4.6 | >4.54 | 3.88< | 4.03 | <4.44 |
| CYP17A1 | 173CP | BT_173 | SKAT | Contrasts | 0.026 | 0.376 | 0.002 | 0.185 | 0.729 | 0.364 | 0.975 | 0.853 | 0.756 | 0.451 | 0.076 | 0.021 |
| CYP17A1 | 173CP | BT_173 | SKAT | N | 11. | 24 | .64 | 29. | 44 | .30 | 21. | 82 | .102 | 56. | 74 | .38 |
| CYP17A1 | 173CP | BT_173 | SKAT | Freq | 99 | | 0.23 | 103 | | 0.50 | 205 | | 0.30 | 168 | | 0.55 |
| CYP2A | 238CP | BT_238 | ANDRO | Effect | 0.700 | | 0.000 | 0.970 | | 0.000 | 1.080 | | 0.950 | −0.480 | | −0.440 |
| CYP2A | 238CP | BT_238 | ANDRO | Effect/ | 0.596 | | 0.000 | 0.769 | | 0.000 | 0.980 | | 0.879 | −0.488 | | −0.445 |
| | | | | RMSE | | | | | | | | | | | | |
| CYP2A | 238CP | BT_238 | ANDRO | Pvalue | 0.245 | | | 0.069 | | | 0.000 | | 0.023 | 0.332 | | 0.444 |
| CYP2A | 238CP | BT_238 | ANDRO | LSM | | −0.33 | <0.37 | | −1.21 | <−0.24 | −1.29< | 0.72 | <0.83 | 0.36> | −0.56 | >−0.6 |
| CYP2A | 238CP | BT_238 | ANDRO | Contrasts | | 0.245 | | | 0.069 | | 0.001 | 0.733 | 0.000 | 0.372 | 0.885 | 0.332 |
| CYP2A | 238CP | BT_238 | ANDRO | N | 0. | 4 | .102 | 0. | 6 | .111 | 4. | 12 | .195 | 1. | 12 | .156 |
| CYP2A | 238CP | BT_238 | ANDRO | Freq | 106 | | 0.02 | 117 | | 0.03 | 211 | | 0.05 | 169 | | 0.04 |
| CYP2A | 238CP | BT_238 | SKAT | Effect | −1.350 | | 0.000 | 0.610 | | 0.000 | 0.520 | | 0.820 | −0.110 | | 0.040 |
| CYP2A | 238CP | BT_238 | SKAT | Effect/ | −1.059 | | 0.000 | 0.525 | | 0.000 | 0.539 | | 0.856 | −0.095 | | 0.035 |
| | | | | RMSE | | | | | | | | | | | | |
| CYP2A | 238CP | BT_238 | SKAT | Pvalue | 0.039 | | | 0.215 | | | 0.034 | | 0.027 | 0.850 | | 0.952 |
| CYP2A | 238CP | BT_238 | SKAT | LSM | | 5.03 | >3.68 | | 4.02 | <4.63 | 3.54< | 4.88 | >4.58 | 4.31> | 4.24 | >4.09 |
| CYP2A | 238CP | BT_238 | SKAT | Contrasts | | 0.030 | | | 0.215 | | 0.017 | 0.288 | 0.034 | 0.954 | 0.867 | 0.850 |
| CYP2A | 238CP | BT_238 | SKAT | N | 0. | 4 | .99 | 0. | 6 | .97 | 4. | 12 | .190 | 1. | 12 | .153 |
| CYP2A | 238CP | BT_238 | SKAT | Freq | 103 | | 0.02 | 103 | | 0.03 | 206 | | 0.05 | 166 | | 0.04 |
| CYP2A | 239CP | BT_239 | ANDRO | Effect | 0.840 | | 0.530 | −0.500 | | −0.250 | 0.059 | | 0.120 | −0.370 | | −0.570 |
| CYP2A | 239CP | BT_239 | ANDRO | Effect/ | 0.726 | | 0.462 | −0.394 | | −0.199 | 0.053 | | 0.107 | −0.385 | | −0.585 |
| | | | | RMSE | | | | | | | | | | | | |
| CYP2A | 239CP | BT_239 | ANDRO | Pvalue | 0.016 | | 0.177 | 0.126 | | 0.541 | 0.698 | | 0.527 | 0.094 | | 0.029 |
| CYP2A | 239CP | BT_239 | ANDRO | LSM | −1.18< | 0.19 | <0.49 | 0.63> | −0.13 | >−0.38 | 0.66< | 0.84 | >0.78 | 0.19> | −0.75 | <−0.56 |
| CYP2A | 239CP | BT_239 | ANDRO | Contrasts | 0.051 | 0.208 | 0.016 | 0.273 | 0.393 | 0.126 | 0.555 | 0.708 | 0.898 | 0.039 | 0.228 | 0.094 |
| CYP2A | 239CP | BT_239 | ANDRO | N | 3. | 35 | .68 | 4. | 25 | .87 | 16. | 99 | .93 | 5. | 55 | .110 |
| CYP2A | 239CP | BT_239 | ANDRO | Freq | 106 | | 0.19 | 116 | | 0.14 | 208 | | 0.31 | 171 | | 0.19 |
| CYP2A | 239CP | BT_239 | SKAT | Effect | 0.096 | | 0.130 | −0.440 | | −0.270 | −0.030 | | 0.073 | −0.570 | | −0.500 |
| CYP2A | 239CP | BT_239 | SKAT | Effect/ | 0.074 | | 0.102 | −0.377 | | −0.235 | −0.031 | | 0.075 | −0.498 | | −0.437 |
| | | | | RMSE | | | | | | | | | | | | |
| CYP2A | 239CP | BT_239 | SKAT | Pvalue | 0.603 | | 0.766 | 0.145 | | 0.472 | 0.820 | | 0.659 | 0.031 | | 0.103 |
| CYP2A | 239CP | BT_239 | SKAT | LSM | 3.53< | 3.76 | >3.72 | 5.37> | 4.66 | >4.5 | 4.58< | 4.63 | >4.52 | 5.17> | 4.09 | >4.02 |
| CYP2A | 239CP | BT_239 | SKAT | Contrasts | 0.771 | 0.895 | 0.803 | 0.259 | 0.541 | 0.145 | 0.871 | 0.470 | 0.820 | 0.047 | 0.714 | 0.031 |
| CYP2A | 239CP | BT_239 | SKAT | N | 3. | 35 | .65 | 4. | 25 | .73 | 16. | 97 | .90 | 5. | 54 | .109 |
| CYP2A | 239CP | BT_239 | SKAT | Freq | 103 | | 0.20 | 102 | | 0.16 | 203 | | 0.32 | 158 | | 0.19 |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| BAC-CT | 224CP | BT_224 | SKAT | Pvalue | 0.841 | | 0.887 | 0.251 | | 0.590 | 0.700 | | 0.085 | 0.305 | | 0.337 |
| BAC-CT | 224CP | BT_224 | SKAT | LSM | 3.8> | 3.72 | =3.72 | 5.22> | 4.68 | >4.52 | 4.42< | 4.77 | >4.51 | 3.69< | 4.12 | >4.09 |
| BAC-CT | 224CP | BT_224 | SKAT | Contrasts | 0.643 | 0.987 | 0.841 | 0.381 | 0.638 | 0.261 | 0.182 | 0.093 | 0.70 | 0.276 | 0.865 | 0.305 |
| BAC-CT | 224CP | BT_224 | SKAT | N | 13. | 45 | .45 | 4. | 35 | .63 | 18. | 61 | .128 | 10. | 68 | .91 |
| BAC-CT | 224CP | BT_224 | SKAT | Freq | 103 | | 0.34 | 102 | | 0.21 | 207 | | 0.23 | 169 | | 0.26 |
| BAC-CT | 225CP | BT_225 | ANDRO | Effect | −0.380 | | 0.053 | 0.150 | | 0.170 | −0.170 | | −0.052 | 0.026 | | −0.042 |
| BAC-CT | 225CP | BT_225 | ANDRO | Effect/ | −0.330 | | 0.046 | 0.114 | | 0.129 | −0.151 | | −0.047 | 0.027 | | −0.043 |
| | | | | RMSE | | | | | | | | | | | | |
| BAC-CT | 225CP | BT_225 | ANDRO | Pvalue | 0.038 | | 0.831 | 0.587 | | 0.619 | 0.242 | | 0.796 | 0.872 | | 0.836 |
| BAC-CT | 225CP | BT_225 | ANDRO | LSM | 0.82> | 0.49 | >0.053 | −0.57< | −0.26 | >−0.28 | 1.07> | 0.85 | >0.74 | −0.61> | −0.62 | <−0.56 |
| BAC-CT | 225CP | BT_225 | ANDRO | Contrasts | −0.365 | 0.07 [symbol] | 0.038 | 0.658 | 0.940 | 0.587 | 0.470 | 0.499 | 0.242 | 0.9 [symbol] | 0.664 | 0.872 |
| BAC-CT | 225CP | BT_225 | ANDRO | N | 13. | 48 | .45 | 7. | 36 | .74 | 17. | 62 | .131 | 10. | 69 | .92 |
| BAC-CT | 225CP | BT_225 | ANDRO | Freq | 106 | | 0.35 | 117 | | 0.21 | 210 | | 0.23 | 171 | | 0.26 |
| BAC-CT | 225CP | BT_225 | SKAT | Effect | −0.041 | | −0.040 | −0.350 | | −0.220 | 0.063 | | 0.310 | 0.210 | | 0.220 |
| BAC-CT | 225CP | BT_225 | SKAT | Effect/ | −0.032 | | −0.031 | −0.298 | | −0.190 | 0.065 | | 0.325 | 0.178 | | 0.192 |
| | | | | RMSE | | | | | | | | | | | | |
| BAC-CT | 225CP | BT_225 | SKAT | Pvalue | 0.841 | | 0.887 | 0.250 | | 0.638 | 0.616 | | 0.078 | 0.286 | | 0.353 |
| BAC-CT | 225CP | BT_225 | SKAT | LSM | 3.8> | 3.72 | =3.72 | 5.22> | 4.65 | 4.52 | 4.39< | 4.77 | >4.51 | 3.69< | 4.12 | >4.1 |
| BAC-CT | 225CP | BT_225 | SKAT | Contrasts | 0.843 | 0.997 | 0.841 | 0.357 | 0.504 | 0.250 | 0.157 | 0.097 | 0.616 | 0.276 | 0.831 | 0.28 [symbol] |
| BAC-CT | 225CP | BT_225 | SKAT | N | 13. | 45 | .45 | 4. | 35 | .63 | 17. | 61 | .127 | 10. | 68 | .90 |
| BAC-CT | 225CP | BT_225 | SKAT | Freq | 103 | | 0.34 | 103 | | 0.21 | 205 | | 0.23 | 168 | | 0.26 |
| BAC-CT | 226CP | BT_225 | ANDRO | Effect | 0.380 | | 0.053 | −0.150 | | 0.170 | 0.170 | | −0.052 | −0.026 | | −0.038 |
| BAC-CT | 226CP | BT_226 | ANDRO | Effect/ | 0.330 | | 0.046 | −0.114 | | 0.129 | 0.151 | | −0.047 | −0.027 | | −0.039 |
| | | | | RMSE | | | | | | | | | | | | |
| BAC-CT | 226CP | BT_226 | ANDRO | Pvalue | 0.038 | | 0.831 | 0.567 | | 0.619 | 0.242 | | 0.796 | 0.873 | | 0.850 |
| BAC-CT | 226CP | BT_226 | ANDRO | LSM | 0.053< | 0.49 | <0.82 | −0.28< | −0.26 | >−0.57 | 0.74< | 0.85 | <1.07 | −0.56> | −0.62 | <−0.61 |
| BAC-CT | 226CP | BT_226 | ANDRO | Contrasts | 0.073 | 0.365 | 0.038 | 0.940 | 0.658 | 0.587 | 0.499 | 0.470 | 0.242 | 0.682 | 0.971 | 0.873 |
| BAC-CT | 226CP | BT_226 | ANDRO | N | 45. | 48 | .13 | 74. | 36 | .7 | 131. | 52 | .17 | 92. | 68 | .10 |
| BAC-CT | 226CP | BT_226 | ANDRO | Freq | 106 | | 0.65 | 117 | | 0.79 | 210 | | 0.77 | 170 | | 0.74 |
| BAC-CT | 226CP | BT_226 | SKAT | Effect | 0.041 | | −0.040 | 0.350 | | −0.220 | −0.063 | | 0.310 | −0.210 | | 0.250 |
| BAC-CT | 226CP | BT_226 | SKAT | Effect/ | 0.032 | | −0.031 | 0.298 | | −0.190 | −0.065 | | 0.325 | −0.180 | | 0.222 |
| | | | | RMSE | | | | | | | | | | | | |
| BAC-CT | 226CP | BT_226 | SKAT | Pvalue | 0.841 | | 0.887 | 0.250 | | 0.538 | 0.616 | | 0.078 | 0.283 | | 0.284 |
| BAC-CT | 226CP | BT_226 | SKAT | LSM | 3.72= | 3.72 | <3.6 | 4.52< | 4.65 | <5.22 | 4.51< | 4.77 | >4.39 | 4.1< | 4.15 | >3.69 |
| BAC-CT | 226CP | BT_226 | SKAT | Contrasts | 0.997 | 0.843 | 0.841 | 0.604 | 0.357 | 0.250 | 0.097 | 0.167 | 0.616 | 0.793 | 0.238 | 0.283 |
| BAC-CT | 226CP | BT_226 | SKAT | N | 45. | 45 | .13 | 63. | 36 | .4 | 127. | 61 | .17 | 90. | 67 | .10 |
| BAC-CT | 226CP | BT_226 | SKAT | Freq | 103 | | 0.66 | 103 | | 0.79 | 205 | | 0.77 | 167 | | 0.74 |
| [symbol] | [symbol] | [symbol] | [symbol] | [symbol] | [symbol] | [symbol] | [symbol] | [symbol] | [symbol] | [symbol] | −0.430 | | 0.000 | −0.095 | | −0.041 |
| [symbol] | [symbol] | [symbol] | [symbol] | [symbol] | 0.020 | | 0.025 | −0.114 | | 0.400 | −0.417 | | 0.000 | −0.083 | | −0.040 |
| CYP2A | 240CP | BT_240 | ANDRO | Pvalue | 0.899 | | 0.900 | 0.821 | | 0.511 | 0.087 | | | 0.391 | | 0.808 |
| CYP2A | 240CP | BT_240 | ANDRO | LSM | −0.67< | −0.61 | >−0.62 | −0.99< | −0.74 | >−1.2 | −0.026> | −0.45 | | −0.54> | −0.67 | >−0.73 |
| CYP2A | 240CP | BT_240 | ANDRO | Contrasts | 0.758 | 0.988 | 0.899 | 0.428 | 0.628 | 0.821 | 0.087 | | | 0.423 | 0.816 | 0.391 |
| CYP2A | 240CP | BT_240 | ANDRO | N | 126. | 67 | .11 | 59. | 9 | .1 | 118. | 20 | .0 | 83. | 65 | .29 |
| 0.284CYP2A | 240CP | BT_240 | ANDRO | Freq | 204 | | 0.78 | 69 | | 0.92 | 139 | | 0.93 | 177 | | 0.65 |
| CYP2A | 240CP | BT_240 | SKAT | Effect | 0.170 | | −0.043 | 0.500 | | −0.850 | −0.190 | | 0.000 | 0.070 | | 0.024 |
| CYP2A | 240CP | BT_240 | SKAT | Effect/ | 0.147 | | −0.036 | 0.489 | | −0.836 | −0.157 | | 0.000 | 0.067 | | 0.023 |
| | | | | RMSE | | | | | | | | | | | | |
| CYP2A | 240CP | BT_240 | SKAT | Pvalue | 0.373 | | 0.680 | 0.386 | | 0.172 | 0.529 | | | 0.597 | | 0.688 |
| CYP2A | 240CP | BT_240 | SKAT | LSM | 4.25< | 4.38 | <4.6 | 4.19> | 3.84 | <5.19 | 3.76> | 3.57 | | 3.12< | 3.22 | <3.26 |
| CYP2A | 240CP | BT_240 | SKAT | Contrasts | 0.469 | 0.591 | 0.373 | 0.338 | 0.213 | 0.336 | 0.529 | | | 0.587 | 0.645 | 0.537 |
| CYP2A | 240CP | BT_240 | SKAT | N | 126. | 66 | .10 | 57. | 9 | .1 | 107. | 19 | .0 | 83. | 66 | .29 |
| CYP2A | 240CP | BT_240 | SKAT | Freq | 202 | | 0.79 | 67 | | 0.92 | 128 | | 0.93 | 178 | | 0.65 |
| CYP2E1 | 152CP | BT_152 | ANDRO | Effect | −0.230 | | 0.300 | 0.500 | | −0.570 | −0.130 | | −0.076 | −0.270 | | 0.130 |
| CYP2E1 | 152CP | BT_152 | ANDRO | Effect/ | −0.163 | | 0.233 | 0.698 | | −0.667 | −0.129 | | −0.074 | −0.262 | | 0.124 |
| | | | | RMSE | | | | | | | | | | | | |
| CYP2E1 | 152CP | BT_152 | ANDRO | Pvalue | 0.172 | | 0.187 | 0.021 | | 0.140 | 0.611 | | 0.838 | 0.130 | | 0.593 |
| CYP2E1 | 152CP | BT_152 | ANDRO | LSM | −0.62< | −0.56 | >−1.09 | −1.02< | −0.99 | <0.18 | −0.057> | −0.27 | >−0.33 | −0.57> | −0.71 | >−1.1 |
| CYP2E1 | 152CP | BT_152 | ANDRO | Contrasts | 0.733 | 0.132 | 0.172 | 0.930 | 0.045 | 0.021 | 0.460 | 0.821 | 0.611 | 0.444 | 0.295 | 0.130 |
| CYP2E1 | 152CP | BT_152 | ANDRO | N | 117. | 75 | .16 | 57. | 9 | .3 | 119. | 15 | .4 | 126. | 41 | .9 |
| CYP2E1 | 152CP | BT_152 | ANDRO | Freq | 208 | | 0.74 | 69 | | 0.89 | 138 | | 0.92 | 176 | | 0.83 |
| CYP2E1 | 152CP | BT_152 | SKAT | Effect | −0.150 | | 0.110 | 0.300 | | −0.960 | 0.110 | | −0.086 | 0.190 | | −0.260 |
| CYP2E1 | 152CP | BT_152 | SKAT | Effect/ | −0.130 | | 0.092 | 0.299 | | −0.953 | 0.090 | | −0.071 | 0.185 | | −0.249 |
| | | | | RMSE | | | | | | | | | | | | |
| CYP2E1 | 152CP | BT_152 | SKAT | Pvalue | 0.330 | | 0.601 | 0.317 | | 0.043 | 0.723 | | 0.846 | 0.284 | | 0.284 |
| CYP2E1 | 152CP | BT_152 | SKAT | LSM | 4.34> | 4.3 | >4.03 | 4.21> | 3.55 | <4.81 | 3.72< | 3.75 | <3.95 | 3.17> | 3.11 | <3.56 |
| CYP2E1 | 152CP | BT_152 | SKAT | Contrasts | 0.799 | 0.420 | 0.330 | 0.088 | 0.069 | 0.317 | 0.949 | 0.775 | 0.723 | 0.724 | 0.238 | 0.284 |
| CYP2E1 | 152CP | BT_152 | SKAT | N | 115. | 75 | .16 | 56. | 8 | .3 | 107. | 15 | .4 | 126. | 42 | .9 |
| CYP2E1 | 152CP | BT_152 | SKAT | Freq | 206 | | 0.74 | 67 | | 0.90 | 126 | | 0.91 | 177 | | 0.83 |
| CYP2E1 | 153CP | BT_153 | ANDRO | Effect | 0.100 | | 0.100 | 0.160 | | 0.130 | −0.065 | | 0.200 | −0.160 | | −0.001 |
| CYP2E1 | 153CP | BT_153 | ANDRO | Effect/ | 0.080 | | 0.078 | 0.185 | | 0.148 | −0.063 | | 0.198 | −0.162 | | −0.001 |
| | | | | RMSE | | | | | | | | | | | | |
| CYP2E1 | 153CP | BT_153 | ANDRO | Pvalue | 0.650 | | 0.661 | 0.304 | | 0.580 | 0.688 | | 0.248 | 0.120 | | 0.995 |
| CYP2E1 | 153CP | BT_153 | ANDRO | LSM | −0.82< | −0.62 | =−0.62 | −1.23< | −0.93 | <−0.9 | −0.12< | 0.022 | >−0.25 | −0.48> | −0.64 | >−0.81 |
| CYP2E1 | 153CP | BT_153 | ANDRO | Contrasts | | 0.933 | 0.550 | 0.345 | | 0.304 | 0.507 | 0.214 | 0.5 | 0.348 | 0.413 | 0.120 |
| CYP2E1 | 153CP | BT_153 | ANDRO | N | 16. | 71 | .121 | 11. | 31 | .27 | 39. | 63 | .36 | 58. | 79 | 0.39 |
| CYP2E1 | 153CP | BT_153 | ANDRO | Freq | 208 | | 0.25 | 69 | | 0.38 | 138 | | 0.51 | 176 | | 0.55 |
| CYP2E1 | 153CP | BT_153 | SKAT | Effect | −0.042 | | −0.084 | −0.004 | | −0.230 | 0.083 | | −0.270 | 0.014 | | −0.011 |
| CYP2E1 | 153CP | BT_153 | SKAT | Effect/ | −0.036 | | −0.071 | −0.004 | | −0.223 | 0.088 | | −0.219 | 0.013 | | −0.010 |
| | | | | RMSE | | | | | | | | | | | | |
| CYP2E1 | 153CP | BT_153 | SKAT | Pvalue | 0.788 | | 0.691 | 0.982 | | 0.388 | 0.674 | | 0.222 | 0.899 | | 0.947 |
| CYP2E1 | 153CP | BT_153 | SKAT | LSM | 4.39> | 4.27 | <4.31 | 4.27> | 4.04 | <4.26 | 3.77> | 3.59 | <3.94 | 3.17< | 3.17 | <3.2 |
| CYP2E1 | 153CP | BT_153 | SKAT | Contrasts | 0.700 | 0.813 | 0.788 | 0.521 | 0.419 | 0.982 | 0.485 | 0.183 | 0.574 | 0.988 | 0.905 | 0.899 |
| CYP2E1 | 153CP | BT_153 | SKAT | N | 16. | 70 | .120 | 11. | 31 | .25 | 34. | 57 | .35 | 58. | 79 | .40 |
| CYP2E1 | 153CP | BT_153 | SKAT | Freq | 206 | | 0.25 | 67 | | 0.40 | 126 | | 0.50 | 177 | | 0.55 |
| CYP2E1 | 158CP | BT_158 | ANDRO | Effect | 0.100 | | 0.100 | 0.230 | | 0.180 | −0.050 | | 0.190 | −0.140 | | 0.019 |
| CYP2E1 | 158CP | BT_158 | ANDRO | Effect/ | 0.080 | | 0.078 | 0.256 | | 0.205 | −0.048 | | 0.187 | −0.143 | | 0.019 |
| | | | | RMSE | | | | | | | | | | | | |
| CYP2E1 | 158CP | BT_158 | ANDRO | Pvalue | 0.649 | | 0.661 | 0.172 | | 0.432 | 0.681 | | 0.279 | 0.169 | | 0.828 |
| CYP2E1 | 158CP | BT_158 | ANDRO | LSM | −0.82< | −0.62 | <−0.62 | −1.35< | −0.94 | <−0.9 | −0.12< | 0.022 | >−0.22 | −0.52> | −0.64 | >−0.81 |
| CYP2E1 | 158CP | BT_158 | ANDRO | Contrasts | 0.568 | 0.990 | 0.549 | 0.211 | 0.851 | 0.172 | 0.600 | 0.2 [symbol] | 0.681 | 0.476 | 0.407 | 0.189 |
| CYP2E1 | 158CP | BT_158 | ANDRO | N | 16. | 72 | .121 | 10. | 30 | .27 | 38 | 63 | .35 | 57. | 79 | .39 |
| CYP2E1 | 158CP | BT_158 | ANDRO | Freq | 209 | | 0.25 | 67 | | 0.37 | 135 | | 0.51 | 175 | | 0.55 |
| CYP2E1 | 158CP | BT_158 | SKAT | Effect | −0.042 | | −0.072 | −0.031 | | −0.260 | 0.081 | | −0.240 | 0.031 | | 0.007 |
| CYP2E1 | 158CP | BT_158 | SKAT | Effect/ | −0.036 | | −0.061 | −0.030 | | −0.250 | 0.066 | | −0.198 | 0.030 | | 0.006 |
| | | | | RMSE | | | | | | | | | | | | |
| CYP2E1 | 158CP | BT_158 | SKAT | Pvalue | 0.788 | | 0.733 | 0.873 | | 0.343 | 0.588 | | 0.278 | 0.774 | | 0.967 |
| CYP2E1 | 158CP | BT_158 | SKAT | LSM | 4.39> | 4.28 | <4.31 | 4.32> | 4.03 | <4.26 | 3.75> | 3.59 | <3.91 | 3.13< | 3.17 | <3.2 |
| CYP2E1 | 158CP | BT_158 | SKAT | Contrasts | 0.727 | 0.867 | 0.788 | 0.448 | 0.420 | 0.873 | 0.553 | 0.227 | 0.668 | 0.837 | 0.905 | 0.774 |
| CYP2E1 | 158CP | BT_158 | SKAT | N | 16. | 71 | .120 | 10. | 30 | .25 | 33. | 57 | .34 | 57. | 79 | .40 |
| CYP2E1 | 158CP | BT_158 | SKAT | Freq | 207 | | 0.25 | 65 | | 0.38 | 124 | | 0.50 | 176 | | 0.55 |
| CYP2E1 | 193CP | BT_193 | ANDRO | Effect | −0.240 | | 0.170 | −0.240 | | 0.200 | 0.081 | | 0.130 | 0.270 | | −0.170 |
| CYP2E1 | 193CP | BT_193 | ANDRO | Effect/ | −0.189 | | 0.134 | −0.260 | | 0.219 | 0.078 | | 0.127 | 0.286 | | −0.181 |
| | | | | RMSE | | | | | | | | | | | | |
| CYP2E1 | 193CP | BT_193 | ANDRO | Pvalue | 0.198 | | 0.480 | 0.172 | | 0.415 | 0.517 | | 0.473 | 0.014 | | 0.282 |
| CYP2E1 | 193CP | BT_193 | ANDRO | LSM | −0.59> | −0.66 | >−1.07 | −0.88> | −0.91 | >−1.35 | −0.24< | −0.028 | >−0.079 | −0.76< | −0.66 | <−0.22 |
| CYP2E1 | 193CP | BT_193 | ANDRO | Contrasts | 0.722 | 0.287 | 0.198 | 0.881 | 0.198 | 0.172 | 0.339 | 0.818 | 0.517 | 0.619 | 0.019 | 0.014 |
| CYP2E1 | 193CP | BT_193 | ANDRO | N | 119. | 68 | .13 | .24 | 28 | .10 | 35. | 60 | .35 | .34 | 67 | .42 |
| CYP2E1 | 193CP | BT_193 | ANDRO | Freq | 200 | | 0.77 | 62 | | 0.61 | 130 | | 0.50 | 143 | | 0.47 |
| CYP2E1 | 193CP | BT_193 | SKAT | Effect | −0.008 | | −0.082 | 0.024 | | −0.340 | −0.069 | | −0.270 | 0.037 | | −0.052 |
| CYP2E1 | 193CP | BT_193 | SKAT | Effect/ | −0.007 | | −0.070 | 0.023 | | −0.323 | −0.055 | | −0.220 | 0.035 | | −0.049 |
| | | | | RMSE | | | | | | | | | | | | |
| CYP2E1 | 193CP | BT_193 | SKAT | Pvalue | 0.963 | | 0.713 | 0.905 | | 0.234 | 0.648 | | 0.234 | 0.782 | | 0.771 |
| CYP2E1 | 193CP | BT_193 | SKAT | LSM | 4.29> | 4.2 | <4.28 | 4.28> | 3.96 | <4.32 | 3.94> | 3.6 | <3.8 | 3.19> | 3.18 | <3.27 |
| CYP2E1 | 193CP | BT_193 | SKAT | Contrasts | 0.615 | 0.835 | 0.983 | 0.298 | 0.352 | 0.905 | 0.206 | 0.470 | 0.648 | 0.946 | 0.872 | 0.782 |
| CYP2E1 | 193CP | BT_193 | SKAT | N | 119. | 67 | .13 | 22. | 28 | .10 | 35. | 54 | .31 | 35. | 67 | .42 |
| CYP2E1 | 193CP | BT_193 | SKAT | Freq | 199 | | 0.77 | 60 | | 0.60 | 120 | | 0.52 | 144 | | 0.48 |
| CYTB5 | 156CP | BT_156 | ANDRO | Effect | 0.480 | | 0.000 | −0.420 | | 0.000 | 0.440 | | 0.000 | −0.370 | | 0.014 |
| CYTB5 | 156CP | BT_156 | ANDRO | Effect/ | 0.380 | | 0.000 | −0.482 | | 0.000 | 0.420 | | 0.000 | −0.365 | | 0.014 |
| | | | | RMSE | | | | | | | | | | | | |
| CYTB5 | 156CP | BT_156 | ANDRO | Pvalue | 0.108 | | | 0.303 | | | 0.358 | | | 0.082 | | 0.959 |
| CYTB5 | 156CP | BT_156 | ANDRO | LSM | −0.69< | −0.21 | | −0.93> | −1.36 | | −0.1< | 0.34 | | −0.54> | −0.89 | >−1.28 |
| CYTB5 | 156CP | BT_156 | ANDRO | Contrasts | 0.108 | | | 0.303 | | | 0.358 | | | 0.079 | 0.397 | 0.082 |
| CYTB5 | 156CP | BT_156 | ANDRO | N | 187. | 20 | .0 | 64. | 5 | .0 | 131. | 5 | .0 | 138. | 31 | .6 |
| CYTB5 | 156CP | BT_156 | ANDRO | Freq | 207 | | 0.95 | 69 | | 0.96 | 136 | | 0.98 | 175 | | 0.88 |
| CYTB5 | 156CP | BT_156 | SKAT | Effect | −0.380 | | 0.000 | −0.320 | | 0.000 | 0.530 | | 0.000 | 0.085 | | −0.032 |
| CYTB5 | 156CP | BT_156 | SKAT | Effect/ | −0.319 | | 0.000 | −0.310 | | 0.000 | 0.440 | | 0.000 | 0.082 | | −0.031 |
| | | | | RMSE | | | | | | | | | | | | |
| CYTB5 | 156CP | BT_156 | SKAT | Pvalue | 0.178 | | | 0.507 | | | 0.388 | | | 0.696 | | 0.911 |
| CYTB5 | 156CP | BT_156 | SKAT | LSM | 4.35> | 3.97 | | 4.18> | 3.86 | | 3.7< | 4.24 | | 3.17< | 3.22 | <3.34 |
| CYTB5 | 156CP | BT_156 | SKAT | Contrasts | 0.176 | | | 0.507 | | | 0.358 | | | 0.799 | 0.802 | 0.696 |
| CYTB5 | 156CP | BT_156 | SKAT | N | 185. | 20 | .0 | 62. | 5 | .0 | 121. | 4 | .0 | 139. | 31 | .6 |
| CYTB5 | 156CP | BT_156 | SKAT | Freq | 205 | | 0.95 | 67 | | 0.96 | 125 | | 0.98 | 176 | | 0.88 |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | 0.217 | | 0.000 | 0.286 | | 0.000 | 1.585 | | 0.000 | 0.647 | | 0.000 |
| CYTB5 | 161CP | BT_161 | ANDRO | Pvalue | 0.485 | | | 0.691 | | | 0.002 | | | 0.269 | | |
| CYTB5 | 161CP | BT_161 | ANDRO | LSM | −0.65< | −0.38 | | −0.97< | −0.72 | | −0.14< | 1.45 | | −0.66< | −0.0097 | |
| CYTB5 | 161CP | BT_161 | ANDRO | Contrasts | 0.485 | | | 0.69 | | | 0.002 | | | 0.268 | | |
| CYTB5 | 161CP | BT_161 | ANDRO | N | 195. | 11 | .0 | 67. | 2 | .0 | 133. | 4 | .0 | 164. | 3 | .0 |
| CYTB5 | 161CP | BT_161 | ANDRO | Freq | 205 | | 0.97 | 69 | | 0.99 | 137 | | 0.99 | 167 | | 0.99 |
| CYTB5 | 161CP | BT_161 | SKAT | Effect | −0.340 | | 0.000 | −0.005 | | 0.000 | 1.550 | | 0.000 | 0.100 | | 0.000 |
| CYTB5 | 161CP | BT_161 | SKAT | Effect/ | −0.286 | | 0.000 | −0.005 | | 0.000 | 1.301 | | 0.000 | 0.102 | | 0.000 |
| | | | | RMSE | | | | | | | | | | | | |
| CYTB5 | 161CP | BT_161 | SKAT | Pvalue | 0.357 | | | 0.994 | | | 0.023 | | | 0.881 | | |
| CYTB5 | 161CP | BT_161 | SKAT | LSM | 4.31> | 3.97 | | 4.16> | 4.15 | | 3.69< | 5.25 | | 3.11< | 3.21 | |
| CYTB5 | 161CP | BT_161 | SKAT | Contrasts | 0.357 | | | 0.994 | | | 0.028 | | | 0.961 | | |
| CYTB5 | 161CP | BT_161 | SKAT | N | 193. | 11 | .0 | 65. | 2 | .0 | 122. | 3 | .0 | 165. | 3 | .0 |
| CYTB5 | 161CP | BT_161 | SKAT | Freq | 204 | | 0.97 | 67 | | 0.99 | 125 | | 0.99 | 168 | | 0.99 |
| SULT1A1 | 140CP | BT_140 | ANDRO | Effect | 0.230 | | −0.077 | 0.170 | | −0.390 | 0.120 | | −0.082 | 0.150 | | −0.230 |
| SULT1A1 | 140CP | BT_140 | ANDRO | Effect/ | 0.181 | | −0.061 | 0.188 | | −0.438 | 0.114 | | −0.078 | 0.140 | | −0.215 |
| | | | | RMSE | | | | | | | | | | | | |
| SULT1A1 | 140CP | BT_140 | ANDRO | Pvalue | 0.124 | | 0.738 | 0.314 | | 0.149 | 0.410 | | 0.894 | 0.425 | | 0.354 |
| SULT1A1 | 140CP | BT_140 | ANDRO | LSM | −0.72< | −0.57 | <−0.26 | −0.93> | −1.15 | <−0.59 | −0.13< | −0.094 | <0.11 | −0.63> | −0.71 | <−0.34 |
| SULT1A1 | 140CP | BT_140 | ANDRO | Contrasts | 0.455 | 0.348 | 0.124 | 0.385 | 0.130 | 0.314 | 0.850 | 0.509 | 0.410 | 0.687 | 0.326 | 0.426 |
| SULT1A1 | 140CP | BT_140 | ANDRO | N | 133. | 54 | .21 | 38. | 18 | .9 | 74. | 46 | .16 | 87. | 54 | .9 |
| SULT1A1 | 140CP | BT_140 | ANDRO | Freq | 208 | | 0.77 | 65 | | 0.72 | 136 | | 0.71 | 150 | | 0.76 |
| SULT1A1 | 140CP | BT_140 | SKAT | Effect | 0.160 | | −0.300 | 0.240 | | −0.260 | −0.070 | | −0.150 | 0.210 | | −0.080 |
| SULT1A1 | 140CP | BT_140 | SKAT | Effect/ | 0.138 | | −0.250 | 0.228 | | −0.252 | −0.057 | | −0.121 | 0.215 | | −0.080 |
| | | | | RMSE | | | | | | | | | | | | |
| SULT1A1 | 140CP | BT_140 | SKAT | Pvalue | 0.240 | | 0.168 | 0.228 | | 0.413 | 0.583 | | 0.559 | 0.222 | | 0.718 |
| SULT1A1 | 140CP | BT_140 | SKAT | LSM | 4.3> | 4.17 | <4.63 | 4.1> | 4.07 | <4.58 | 3.83> | 3.61 | <3.69 | 3.08< | 3.21 | <3.51 |
| SULT1A1 | 140CP | BT_140 | SKAT | Contrasts | 0.482 | 0.133 | 0.240 | 0.534 | 0.249 | 0.228 | 0.362 | 0.829 | 0.693 | 0.437 | 0.414 | 0.222 |
| SULT1A1 | 140CP | BT_140 | SKAT | N | 132. | 53 | .21 | 37. | 17 | .9 | 64. | 45 | .15 | .88 | 54 | .9 |
| SULT1A1 | 140CP | BT_140 | SKAT | Freq | 206 | | 0.77 | 63 | | 0.72 | 124 | | 0.70 | 151 | | 0.76 |
| SULT1A1 | 141CP | BT_141 | ANDRO | Effect | −0.160 | | −0.120 | −0.170 | | −0.610 | −0.380 | | −0.530 | 0.380 | | 0.660 |
| SULT1A1 | 141CP | BT_141 | ANDRO | Effect/ | −0.129 | | −0.096 | −0.191 | | −0.682 | −0.383 | | −0.507 | 0.367 | | 0.639 |
| | | | | RMSE | | | | | | | | | | | | |
| SULT1A1 | 141CP | BT_141 | ANDRO | Pvalue | 0.456 | | 0.651 | 0.708 | | 0.435 | 0.311 | | 0.289 | 0.486 | | 0.238 |
| SULT1A1 | 141CP | BT_141 | ANDRO | LSM | −0.33> | −0.62 | >−0.65 | 0.62> | −1.39 | <−0.96 | 0.67> | −0.23 | <−0.082 | −1.42< | −0.3 | >−0.66 |
| SULT1A1 | 141CP | BT_141 | ANDRO | Contrasts | 0.528 | 0.827 [symbol] | 0.456 | 0.478 | 0.4 [symbol] | 0.264 [symbol] | 0.264 | 0.682 | 0.311 | 0.324 | 0.197 | 0.485 |
| SULT1A1 | 141CP | BT_141 | ANDRO | N | 9. | 56 | .134 | 1. | 2 | .66 | 2. | 10 | .124 | 1. | 27 | .141 |
| SULT1A1 | 141CP | BT_141 | ANDRO | Freq | 208 | | 0.20 | 69 | | 0.03 | 136 | | 0.05 | 169 | | 0.09 |
| SULT1A1 | 141CP | BT_141 | SKAT | Effect | 0.350 | | 0.081 | 0.380 | | −0.055 | −0.520 | | −0.260 | −0.460 | | −0.600 |
| SULT1A1 | 141CP | BT_141 | SKAT | Effect/ | 0.295 | | 0.069 | 0.367 | | −0.053 | −0.428 | | −0.212 | −0.442 | | −0.570 |
| | | | | RMSE | | | | | | | | | | | | |
| SULT1A1 | 141CP | BT_141 | SKAT | Pvalue | 0.088 | | [symbol] | 0.469 | | 0.95 | [symbol] | | 0.721 | 0.380 | | 0.290 |
| SULT1A1 | 141CP | BT_141 | SKAT | LSM | 3.72< | 4.15 | <4.42 | 3.43< | 3.75 | <4.18 | 4.75> | 3.97 | >3.71 | 4.11> | 3.05 | <3.18 |
| SULT1A1 | 141CP | BT_141 | SKAT | Contrasts | 0.307 | 0.138 | 0.088 | 0.799 | 0.560 | 0.469 | 0.543 | 0.515 | 0. [symbol] | 0.322 | 0.542 | 0.380 |
| SULT1A1 | 141CP | BT_141 | SKAT | N | 9. | 64 | .134 | 1. | 2 | .64 | 1. | 10 | .113 | 1. | 27 | .142 |
| SULT1A1 | 141CP | BT_141 | SKAT | Freq | 207 | | 0.20 | 67 | | 0.03 | 124 | | 0.05 | 170 | | 0.09 |
| SULT1A1 | 162CP | BT_162 | ANDRO | Effect | −0.160 | | 0.200 | −0.009 | | −0.320 | −0.083 | | −0.310 | −0.130 | | −0.072 |
| SULT1A1 | 162CP | BT_162 | ANDRO | Effect/ | −0.127 | | 0.157 | −0.010 | | −0.358 | −0.080 | −0.304 | −0.304 | −0.129 | | −0.071 |
| | | | | RMSE | | | | | | | | | | | | |
| SULT1A1 | 162CP | BT_162 | ANDRO | Pvalue | 0.161 | | 0.268 | 0.948 | | 0.173 | 0.634 | | 0.088 | 0.260 | | 0.665 |
| SULT1A1 | 162CP | BT_162 | ANDRO | LSM | −0.56< | −0.52 | >−0.88 | −0.64> | −1.17 | <0.66 | 0.17> | −0.23 | <0.0051 | −0.47> | −0.67 | >−0.73 |
| SULT1A1 | 162CP | BT_162 | ANDRO | Contrasts | 0.861 [symbol] | 0.095 [symbol] | 0.151 | 0.277 | 0.205 | [symbol] | 0.107 | 0.258 | 0.534 | 0.371 | 0.738 | 0.260 |
| SULT1A1 | 162CP | BT_162 | ANDRO | N | 63. | 65 | .59 | 14. | 24 | .31 | 24. | 70 | .41 | 28. | 74 | .62 |
| SULT1A1 | 162CP | BT_162 | ANDRO | Freq | 207 | | 0.51 | 69 | | 0.38 | 135 | | 0.44 | 164 | | 0.40 |
| SULT1A1 | 162CP | BT_162 | SKAT | Effect | 0.061 | | −0.170 | −0.080 | | 0.029 | −0.003 | | −0.044 | −0.150 | | −0.160 |
| SULT1A1 | 162CP | BT_162 | SKAT | Effect/ | 0.053 | | −0.146 | −0.077 | | 0.028 | −0.003 | | −0.036 | −0.142 | | −0.154 |
| | | | | RMSE | | | | | | | | | | | | |
| SULT1A1 | 162CP | BT_162 | SKAT | Pvalue | 0.559 | | 0.308 | 0.643 | | 0.815 | 0.964 | | 0.844 | 0.212 | | 0.348 |
| SULT1A1 | 162CP | BT_162 | SKAT | LSM | 4.32> | 4.21 | <4.45 | 4.25> | 4.2 | >4.09 | 3.74> | 3.69 | <3.73 | 3.42> | 3.11 | <3.12 |
| SULT1A1 | 162CP | BT_162 | SKAT | Contrasts | 0.577 | 0.212 | 0.559 | 0.889 | 0.703 | 0.643 | 0.873 | 0.875 | 0.964 | 0.184 | 0.946 | 0.212 |
| SULT1A1 | 162CP | BT_162 | SKAT | N | 63. | 82 | .60 | 13. | 23 | .31 | 23. | 67 | .34 | .28 | 74 | .63 |
| SULT1A1 | 162CP | BT_162 | SKAT | Freq | 205 | | 0.51 | 67 | | 0.37 | 124 | | 0.46 | 165 | | 0.39 |
| SULT1A1 | 171CP | BT_171 | ANDRO | Effect | −0.100 | | 0.022 | −0.044 | | −0.310 | 0.032 | | −0.120 | −0.120 | | −0.140 |
| SULT1A1 | 171CP | BT_171 | ANDRO | Effect/ | −0.080 | | 0.017 | −0.050 | | −0.345 | 0.031 | | −0.115 | −0.115 | | −0.133 |
| | | | | RMSE | | | | | | | | | | | | |
| SULT1A1 | 171CP | BT_171 | ANDRO | Pvalue | 0.379 | | 0.918 | 0.767 | | 0.208 | 0.818 | | 0.527 | 0.403 | | 0.459 |
| SULT1A1 | 171CP | BT_171 | ANDRO | LSM | −0.51> | −0.59 | >−0.71 | −0.8> | −1.15 | <−0.89 | −0.088> | −0.18 | <−0.025 | −0.39> | −0.65 | <−0.63 |
| SULT1A1 | 171CP | BT_171 | ANDRO | Contrasts | 0.768 | 0.567 | 0.379 | 0.275 | 0.284 | 0.767 | 0.742 | 0.436 | 0.816 | 0.370 | 0.911 | 0.403 |
| SULT1A1 | 171CP | BT_171 | ANDRO | N | 41. | 49 | .118 | 12. | 22 | .34 | 20. | 64 | .52 | 16. | 76 | .80 |
| SULT1A1 | 171CP | BT_171 | ANDRO | Freq | 208 | | 0.31 | 68 | | 0.34 | 136 | | 0.38 | 172 | | 0.31 |
| SULT1A1 | 171CP | BT_171 | SKAT | Effect | −0.110 | | −0.260 | −0.130 | | 0.058 | 0.100 | | −0.110 | −0.280 | | −0.240 |
| SULT1A1 | 171CP | BT_171 | SKAT | Effect/ | −0.096 | | −0.224 | −0.130 | | 0.056 | 0.082 | | −0.086 | −0.270 | | −0.235 |
| | | | | RMSE | | | | | | | | | | | | |
| SULT1A1 | 171CP | BT_171 | SKAT | Pvalue | 0.291 | | 0.190 | 0.443 | | 0.840 | 0.650 | | 0.648 | 0.0 [symbol] | | 0.189 |
| SULT1A1 | 171CP | BT_171 | SKAT | LSM | 4.52> | 4.15 | <4.3 | 4.32> | 4.24 | >4.05 | 3.65= | 3.65 | <3.86 | 3.67> | 3.15 | >3.11 |
| SULT1A1 | 171CP | BT_171 | SKAT | Contrasts | 0.133 | 0.455 | 0.291 | 0.839 | 0.506 | 0.443 | 0.987 | 0.395 | 0.550 | 0.068 | 0.828 | 0.050 |
| SULT1A1 | 171CP | BT_171 | SKAT | N | 41. | 48 | .117 | 12. | 21 | .34 | 19. | 61 | .44 | 16. | 76 | .81 |
| SULT1A1 | 171CP | BT_171 | SKAT | Freq | 206 | | 0.32 | 67 | | 0.34 | 124 | | 0.40 | 173 | | 0.31 |
| BAC-CT | 223CP | BT_223 | ANDRO | Effect | −0.032 | | 0.180 | −0.470 | | −0.150 | 0.034 | | −0.066 | −0.150 | | 0.130 |
| BAC-CT | 223CP | BT_223 | ANDRO | Effect/ | −0.026 | | 0.140 | −0.582 | | −0.184 | 0.032 | | −0.063 | −0.150 | | 0.128 |
| | | | | RMSE | | | | | | | | | | | | |
| BAC-CT | 223CP | BT_223 | ANDRO | Pvalue | 0.856 | | 0.450 | 0.001 | | 0.453 | 0.899 | | 0.831 | 0.177 | | 0.420 |
| BAC-CT | 223CP | BT_223 | ANDRO | LSM | −0.66< | −0.52 | >−0.73 | −0.38 | −1 | >−1.32 | −0.079> | −0.11 | <−0.012 | −0.56> | −0.58 | >−0.56 |
| BAC-CT | 223CP | BT_223 | ANDRO | Contrasts | 0.448 | 0.672 | 0.856 | 0.015 | 0.167 | 0.001 | 0.867 | 0.856 | 0.899 | 0.698 | 0.204 | 0.177 |
| BAC-CT | 223CP | BT_223 | ANDRO | N | 120. | 70 | .14 | 16. | 32 | .20 | 89. | 45 | .4 | 70. | 77 | .29 |
| BAC-CT | 223CP | BT_223 | ANDRO | Freq | 204 | | 0.76 | 68 | | 0.47 | 138 | | 0.81 | 176 | | 0.62 |
| BAC-CT | 223CP | BT_223 | SKAT | Effect | 0.220 | | −0.075 | 0.082 | | 0.110 | −0.350 | | 0.400 | −0.068 | | 0.170 |
| BAC-CT | 223CP | BT_223 | SKAT | Effect/ | 0.183 | | −0.063 | 0.079 | | 0.104 | −0.284 | | 0.331 | −0.065 | | 0.165 |
| | | | | RMSE | | | | | | | | | | | | |
| BAC-CT | 223CP | BT_223 | SKAT | Pvalue | 0.212 | | 0.740 | 0.646 | | 0.676 | 0.336 | | 0.323 | 0.558 | | 0.298 |
| BAC-CT | 223CP | BT_223 | SKAT | LSM | 4.22< | 4.36 | <4.66 | 4.03< | 4.22 | >4.19 | 3.73< | 3.79 | >3.04 | 3.16< | 3.26 | >3.02 |
| BAC-CT | 223CP | BT_223 | SKAT | Contrasts | 0.429 | 0.418 | 0.212 | 0. [symbol] | 0.931 | 0.648 | 0. [symbol] | 0.306 | 0.336 | 0.544 | 0.292 | 0. [symbol] |
| BAC-CT | 223CP | BT_223 | SKAT | N | 120. | 69 | .13 | 15. | 31 | .20 | 83. | 40 | .3 | 70. | 78 | .29 |
| BAC-CT | 223CP | BT_223 | SKAT | Freq | 202 | | 0.76 | 56 | | 0.46 | 126 | | 0.82 | 177 | | 0.62 |
| BAC-CT | 224CP | BT_224 | ANDRO | Effect | 0.032 | | 0.110 | 0.420 | | −0.100 | −0.034 | | −0.066 | 0.130 | | 0.110 |
| BAC-CT | 224CP | BT_224 | ANDRO | Effect/ | 0.025 | | 0.087 | 0.509 | | −0.120 | −0.032 | | −0.063 | 0.131 | | 0.113 |
| | | | | RMSE | | | | | | | | | | | | |
| BAC-CT | 224CP | BT_224 | ANDRO | Pvalue | 0.858 | | 0.637 | 0.003 | | 0.621 | 0.899 | | 0.831 | 0.230 | | 0.478 |
| BAC-CT | 224CP | BT_224 | ANDRO | LSM | −0.73< | −0.58 | >−0.66 | −1.32< | −1 | <−0.47 | −0.012> | −0.11 | <−0.079 | −0.82< | −0.58 | <−0.56 |
| BAC-CT | 224CP | BT_224 | ANDRO | Contrasts | 0.702 | 0.679 | 0.859 | 0.177 | 0.040 | 0.003 | 0.855 | 0.657 | 0.889 | 0.259 | 0.910 | 0.230 |
| BAC-CT | 224CP | BT_224 | ANDRO | N | 14. | 73 | .120 | 20. | 32 | .17 | 4. | 45 | .89 | 30. | 76 | .70 |
| BAC-CT | 224CP | BT_224 | ANDRO | Freq | 207 | | 0.24 | 69 | | 0.52 | 138 | | 0.19 | 176 | | 0.39 |
| BAC-CT | 224CP | BT_224 | SKAT | Effect | −0.220 | | −0.070 | −0.100 | | 0.130 | 0.350 | | 0.400 | 0.049 | | 0.160 |
| [symbol] | [symbol] | [symbol] | [symbol] | [symbol] | 0.373 | | 0.602 | −0.137 | | 0.000 | 0.689 | | 0.636 | 0.136 | | 0.000 |
| 3_alfa-HSD | 157CP | BT_157 | ANDRO | Pvalue | 0.457 | | 0.274 | 0.818 | | | 0.172 | | 0.247 | 0.373 | | |
| 3_alfa-HSD | 157CP | BT_157 | ANDRO | LSM | −1.61< | −0.37 | >−0.66 | | −0.87 | >−1 | −1.5< | −0.12 | <−0.069 | | −0.68 | <−0.55 |
| 3_alfa-HSD | 157CP | BT_157 | ANDRO | Contrasts | 0.342 | 0.332 | 0.457 | | 0. [symbol] | | 0.198 | 0.824 | 0.172 | | 0.373 | |
| 3_alfa-HSD | 157CP | BT_157 | ANDRO | N | 1. | 20 | .188 | 0. | 18 | .51 | 1. | 21 | .115 | 0. [symbol] | 101 | .75 |
| 3_alfa-HSD | 157CP | BT_157 | ANDRO | Freq | 209 | | 0.05 | 69 | | 0.13 | 137 | | 0.08 | 176 | | 0.29 |
| 3_alfa-HSD | 157CP | BT_157 | SKAT | Effect | 0.460 | | 0.770 | −0.580 | | 0.000 | 1.030 | | 0.800 | 0.082 | | 0.000 |
| 3_alfa-HSD | 157CP | BT_157 | SKAT | Effect/ | 0.387 | | 0.656 | −0.582 | | 0.000 | 0.856 | | 0.666 | 0.078 | | 0.000 |
| | | | | RMSE | | | | | | | | | | | | |
| 3_alfa-HSD | 157CP | BT_157 | SKAT | Pvalue | 0.441 | | 0.233 | 0.038 | | | 0.091 | | 0.230 | 0.607 | | |
| 3_alfa-HSD | 157CP | BT_157 | SKAT | LSM | 3.37< | 4.59 | >4.28 | | 4.58 | >4 | 1.73< | 3.57 | <3.8 | | 3.14 | <3.22 |
| 3_alfa-HSD | 157CP | BT_157 | SKAT | Contrasts | 0.310 | 0.254 | 0.441 | | 0.038 | | 0.140 | 0.446 | 0.091 | | 0.607 | |
| 3_alfa-HSD | 157CP | BT_157 | SKAT | N | 1. | 20 | .186 | 0. | 18 | .49 | 1. | 19 | .105 | 0. | 102 | .75 |
| 3_alfa-HSD | 157CP | BT_157 | SKAT | Freq | 207 | | 0.05 | 67 | | 0.13 | 125 | | 0.08 | 177 | | 0.29 |
| 3_beta_HSD | 221CP | BT_221 | ANDRO | Effect | 0.140 | | 0.260 | −0.400 | | −0.440 | −0.230 | | −0.550 | −0.400 | | −0.650 |
| 3_beta_HSD | 221CP | BT_221 | ANDRO | Effect/ | 0.107 | | 0.204 | −0.478 | | −0.528 | −0.229 | | −0.539 | −0.396 | | −0.649 |
| | | | | RMSE | | | | | | | | | | | | |
| 3_beta_HSD | 221CP | BT_221 | ANDRO | Pvalue | 0.266 | | 0.153 | 0.033 | | 0.0 [symbol] | 0.157 | | 0.014 | 0.177 | | 0.052 |
| 3_beta_HSD | 221CP | BT_221 | ANDRO | LSM | −0.89< | −0.49 | >−0.62 | −0.24> | −1.09 | <−1.04 | 0.45> | −0.33 | <−0.015 | 0.23> | −0.84 | <−0.58 |
| 3_beta_HSD | 221CP | BT_221 | ANDRO | Contrasts | 0.088 | 0.648 | 0.266 | 0.035 | 0.857 | 0.033 | 0.023 | 0.082 | 0.157 | 0.083 | 0.160 | 0.177 |
| 3_beta_HSD | 221CP | BT_221 | ANDRO | N | 47. | 91 | .66 | 6. | 20 | .40 | 11. | 48 | .79 | 3. | 41 | .128 |
| 3_beta_HSD | 221CP | BT_221 | ANDRO | Freq | 204 | | 0.45 | 66 | | 0.24 | 138 | | 0.25 | 172 | | 0.14 |
| 3_beta_HSD | 221CP | BT_221 | SKAT | Effect | −0.190 | | −0.098 | −0.430 | | −0.130 | −0.230 | | −0.260 | −0.460 | | −0.700 |
| 3_beta_HSD | 221CP | BT_221 | SKAT | Effect/ | −0.162 | | −0.082 | −0.425 | | −0.130 | −0.193 | | −0.217 | −0.438 | | −0.675 |
| | | | | RMSE | | | | | | | | | | | | |
| 3_beta_HSD | 221CP | BT_221 | SKAT | Pvalue | 0.095 | | 0.563 | 0.057 | | 0.683 | 0.257 | | 0.333 | 0.135 | | 0.043 |
| 3_beta_HSD | 221CP | BT_221 | SKAT | LSM | 4.56> | 4.27 | >4.18 | 4.83> | 4.27 | >3.97 | 4.18> | 3.68 | <3.71 | 4.15> | 2.99 | <3.24 |
| 3_beta_HSD | 221CP | BT_221 | SKAT | Contrasts | 0.179 | 0.628 | 0.095 | 0.241 | 0.296 | 0.057 | 0.241 | 0.897 | 0.257 | 0.0 [symbol] | 0.188 | 0.135 |
| 3_beta_HSD | 221CP | BT_221 | SKAT | N | 46. | 91 | .65 | 6. | 19 | .39 | 10. | 47 | .69 | 3. | 41 | .129 |
| 3_beta_HSD | 221CP | BT_221 | SKAT | Freq | 202 | | 0.45 | 64 | | 0.24 | 126 | | 0.27 | 173 | | 0.14 |
| 3_beta_HSD | 222CP | BT_222 | ANDRO | Effect | −0.030 | | 0.130 | −0.300 | | −0.150 | 0.025 | | −0.180 | −0.008 | | −0.071 |
| 3_beta_HSD | 222CP | BT_222 | ANDRO | Effect/ | −0.024 | | 0.104 | −0.342 | | −0.173 | 0.024 | | −0.177 | −0.007 | | −0.069 |
| | | | | RMSE | | | | | | | | | | | | |
| 3_beta_HSD | 222CP | BT_222 | ANDRO | Pvalue | 0.801 | | 0.489 | 0.049 | | 0.518 | 0.847 | | 0.325 | 0.955 | | 0.701 |
| 3_beta_HSD | 222CP | BT_222 | ANDRO | LSM | −0.66< | −0.56 | >−0.72 | −0.52> | −0.97 | >−1.12 | −0.025> | −0.18 | <0.024 | −0.59> | −0.67 | <−0.6 |
| 3_beta_HSD | 222CP | BT_222 | ANDRO | Contrasts | 0.609 | 0.514 | 0.801 | 0.150 | 0.539 | 0.048 | 0.418 | 0.409 | 0.847 | 0.775 | 0.706 | 0.955 |
| 3_beta_HSD | 222CP | BT_222 | ANDRO | N | 92. | 74 | .41 | 12. | 24 | .30 | 54. | 59 | .24 | 18. | 64 | .91 |
| 3_beta_HSD | 222CP | BT_222 | ANDRO | Freq | 207 | | 0.62 | 66 | | 0.36 | 137 | | 0.61 | 173 | | 0.28 |
| 3_beta_HSD | 222CP | BT_222 | SKAT | Effect | −0.190 | | −0.030 | −0.091 | | 0.020 | 0.045 | | 0.250 | −0.089 | | −0.340 |
| 3_beta_HSD | 222CP | BT_222 | SKAT | Effect/ | −0.158 | | −0.025 | −0.087 | | 0.019 | 0.037 | | 0.207 | −0.085 | | −0.330 |
| | | | | RMSE | | | | | | | | | | | | |
| 3_beta_HSD | 222CP | BT_222 | SKAT | Pvalue | 0.098 | | 0.857 | 0.626 | | 0.945 | 0.780 | | 0.274 | 0.510 | | 0.088 |
| 3_beta_HSD | 222CP | BT_222 | SKAT | LSM | 4.45> | 4.23 | >4.08 | 4.27> | 4.2 | >4.09 | 3.59< | 3.88 | >3.68 | 3.44> | 3.01 | <3.26 |
| 3_beta_HSD | 222CP | BT_222 | SKAT | Contrasts | 0.245 | 0.496 | 0.0 [symbol] | 0.853 | 0.703 | 0.626 | 0.213 | 0.518 | 0.780 | 0.122 | 0.135 | 0.510 |
| 3_beta_HSD | 222CP | BT_222 | SKAT | N | 90. | 74 | .41 | 11. | 24 | .29 | 50. | 56 | .20 | 18. | 64 | .92 |
| 3_beta_HSD | 222CP | BT_222 | SKAT | Freq | 205 | | 0.62 | 64 | | 0.36 | 126 | | 0.62 | 174 | | 0.29 |
| CYP17A1 | 173CP | BT_173 | ANDRO | Effect | 0.160 | | −0.061 | 0.410 | | −0.068 | 0.310 | | 0.130 | 0.150 | | 0.200 |
| CYP17A1 | 173CP | BT_173 | ANDRO | Effect/ | 0.129 | | −0.048 | 0.483 | | −0.080 | 0.308 | | 0.126 | 0.149 | | 0.206 |
| | | | | RMSE | | | | | | | | | | | | |
| CYP17A1 | 173CP | BT_173 | ANDRO | Pvalue | 0.210 | | 0.753 | 0.008 | | 0.0788 | 0.009 | | 0.541 | 0.213 | | 0.208 |
| CYP17A1 | 173CP | BT_173 | ANDRO | LSM | −0.83< | −0.73 | <−0.5 | −1.19< | −0.85 | <−0.37 | −0.54< | −0.11 | <0.079 | −0.86< | −0.51 | >−0.57 |
| CYP17A1 | 173CP | BT_173 | ANDRO | Contrasts | 0.700 | 0.248 | 0.210 | 0.170 | 0.151 | 0.006 | 0.100 | 0.376 | 0.009 | 0.112 | 0.748 | 0.213 |
| CYP17A1 | 173CP | BT_173 | ANDRO | N | 32. | 79 | .94 | 39. | 17 | .11 | 25. | 35 | .74 | 27. | 82 | .51 |
| CYP17A1 | 173CP | BT_173 | ANDRO | Freq | 205 | | 0.35 | 67 | | 0.71 | 134 | | 0.32 | 160 | | 0.43 |
| CYP17A1 | 173CP | BT_173 | SKAT | Effect | −0.180 | | −0.063 | −0.320 | | 0.460 | −0.290 | | −0.310 | 0.002 | | 0.370 |
| CYP17A1 | 173CP | BT_173 | SKAT | Effect/ | −0.151 | | −0.054 | −0.323 | | 0.468 | −0.240 | | −0.253 | 0.002 | | 0.350 |
| | | | | RMSE | | | | | | | | | | | | |
| CYP17A1 | 173CP | BT_173 | SKAT | Pvalue | 0.144 | | 0.724 | 0.0 [symbol] | | 0.135 | 0.043 | | 0.23 [symbol] | 0. [symbol] | | 0.032 |
| CYP17A1 | 173CP | BT_173 | SKAT | LSM | 4.53> | 4.29 | >4.18 | 4.19< | 4.34 | >3.55 | 4.19> | 3.59 | <3.6 | 2.99< | 3.36 | >2.99 |
| CYP17A1 | 173CP | BT_173 | SKAT | Contrasts | 0.330 | 0.628 | 0.144 | 0.635 | 0.050 | 0.083 | 0.067 | 0.963 | 0.0 [symbol] | 0.115 | 0.852 | 0.988 |
| CYP17A1 | 173CP | BT_173 | SKAT | N | 32. | 80 | .91 | 39. | 15 | .11 | 25. | 32 | .65 | .27 | 82 | .52 |
| CYP17A1 | 173CP | BT_173 | SKAT | Freq | 203 | | 0.35 | 65 | | 0.72 | 123 | | 0.33 | 161 | | 0.42 |
| CYP2A | 238CP | BT_238 | ANDRO | Effect | −0.270 | | −0.110 | −0.097 | | 0.000 | −0.005 | | 0.000 | −0.270 | | −0.290 |
| CYP2A | 238CP | BT_238 | ANDRO | Effect/ | −0.209 | | −0.087 | −0.110 | | 0.000 | −0.005 | | 0.000 | −0.266 | | −0.284 |
| | | | | RMSE | | | | | | | | | | | | |
| CYP2A | 238CP | BT_238 | ANDRO | Pvalue | 0.223 | | 0.717 | 0.748 | | | 0.989 | | | 0.364 | | 0.388 |
| CYP2A | 238CP | BT_238 | ANDRO | LSM | −0.15> | −0.53 | >−0.69 | | −0.88 | >−0.98 | | −0.083 | >−0.088 | −0.08> | −0.64 | <−0.62 |
| CYP2A | 238CP | BT_238 | ANDRO | Contrasts | 0.428 | 0.507 | 0.223 | | 0.748 | | | 0.989 | | 0.357 | 0.913 | 0.384 |
| CYP2A | 238CP | BT_238 | ANDRO | N | 9. | 36 | .163 | 0. | 10 | .59 | 0. | 10 | .128 | 3. | 46 | .127 |
| CYP2A | 238CP | BT_238 | ANDRO | Freq | 206 | | 0.13 | 69 | | 0.07 | 138 | | 0.04 | 176 | | 0.15 |
| CYP2A | 238CP | BT_238 | SKAT | Effect | −0.310 | | −0.150 | −0.790 | | 0.000 | −0.005 | | 0.000 | 0.048 | | 0.190 |
| CYP2A | 238CP | BT_238 | SKAT | Effect/ | −0.262 | | −0.129 | −0.798 | | 0.000 | −0.004 | | 0.000 | 0.046 | | 0.180 |
| | | | | RMSE | | | | | | | | | | | | |
| CYP2A | 238CP | BT_238 | SKAT | Pvalue | 0.127 | | 0.592 | 0.023 | | | 0.990 | | | 0.876 | | 0.582 |
| CYP2A | 238CP | BT_238 | SKAT | LSM | 4.66> | 4.4 | >4.25 | | 4.83 | >4.04 | | 3.74 | >3.73 | 3.06< | 3.3 | >3.16 |
| CYP2A | 238CP | BT_238 | SKAT | Contrasts | 0.295 | 0.478 | 0.127 | | 0.023 | | | 0.990 | | 0.704 | 0.436 | 0. [symbol] |
| CYP2A | 238CP | BT_238 | SKAT | N | 9. | 35 | .162 | 0. | 10 | .57 | 0. | 10 | .116 | 3. | 46 | .128 |
| CYP2A | 238CP | BT_238 | SKAT | Freq | 206 | | 0.13 | 67 | | 0.07 | 126 | | 0.04 | 177 | | 0.15 |
| CYP2A | 239CP | BT_239 | ANDRO | Effect | 0.190 | | 0.130 | −0.360 | | −0.071 | 0.046 | | −0.160 | −0.060 | | −0.064 |
| CYP2A | 239CP | BT_239 | ANDRO | Effect/ | 0.148 | | 0.101 | −0.415 | | −0.081 | 0.044 | | −0.160 | −0.058 | | −0.063 |
| | | | | RMSE | | | | | | | | | | | | |
| CYP2A | 239CP | BT_239 | ANDRO | Pvalue | 0.121 | | 0.504 | 0.171 | | 0.820 | 0.741 | | 0.396 | 0.679 | | 0.748 |
| CYP2A | 239CP | BT_239 | ANDRO | LSM | −0.93< | −0.61 | <−0.55 | −0.39> | −0.82 | >−1.11 | −0.078> | −0.2 | <0.013 | −0.51> | −0.63 | =−0.63 |
| CYP2A | 239CP | BT_239 | ANDRO | Contrasts | 0.211 | 0.766 | 0.121 | 0.418 | 0.188 | 0.171 | 0.670 | 0.268 | 0.741 | 0.680 | 0.978 | 0.873 |
| CYP2A | 239CP | BT_239 | ANDRO | N | 39. | 72 | .94 | 3. | 27 | .39 | 18. | 58 | .62 | 15. | 52 | .110 |
| CYP2A | 239CP | BT_239 | ANDRO | Freq | 205 | | 0.37 | 69 | | 0.24 | 138 | | 0.34 | 177 | | 0.23 |
| CYP2A | 239CP | BT_239 | SKAT | Effect | 0.099 | | −0.220 | 0.420 | | 0.076 | 0.160 | | −0.310 | −0.029 | | −0.360 |
| CYP2A | 239CP | BT_239 | SKAT | Effect/ | 0.084 | | −0.186 | 0.415 | | 0.075 | 0.135 | | −0.256 | −0.028 | | −0.350 |
| | | | | RMSE | | | | | | | | | | | | |
| CYP2A | 239CP | BT_239 | SKAT | Pvalue | 0.383 | | 0.223 | 0.171 | | 0.838 | 0.356 | | 0.198 | 0.839 | | 0.073 |
| CYP2A | 239CP | BT_239 | SKAT | LSM | 4.26> | 4.14 | <4.46 | 3.46< | 3.98 | <4.32 | 3.66> | 3.51 | <3.98 | 3.33> | 2.94 | <3.28 |
| CYP2A | 239CP | BT_239 | SKAT | Contrasts | 0.611 | 0.087 | 0.383 | 0.425 | 0.189 | 0.171 | 0.678 | 0.042 | 0.356 | 0.19 | 0.056 | 0.839 |
| CYP2A | 239CP | BT_239 | SKAT | N | 38. | 72 | .93 | 3. | 25 | .39 | 15. | 56 | .55 | 15. | 53 | .110 |
| CYP2A | 239CP | BT_239 | SKAT | Freq | 203 | | 0.36 | 67 | | 0.23 | 126 | | 0.34 | 178 | | 0.23 |
| [symbol] | [symbol] | [symbol] | [symbol] | [symbol] | [symbol] | [symbol] | [symbol] | [symbol] | [symbol] | [symbol] | [symbol] | [symbol] | 0.331 | 0.047 | | 0.154 |
| [symbol] | [symbol] | [symbol] | [symbol] | Pvalue | 0.210 | | 0.751 | 0.561 | | 0.818 | 0.338 | | 0.323 | 0.688 | | 0.332 |
| BAC-CT | 224CP | BT_224 | SKAT | LSM | 4.66> | 4.37 | >4.22 | 4.19< | 4.22 | >3.99 | 3.04< | 3.79 | >3.73 | 3.06< | 3.27 | >3.16 |
| BAC-CT | 224CP | BT_224 | SKAT | Contrasts | 0.421 | 0.406 | 0.210 | 0.930 | 0.475 | 0.561 | 0.306 | 0.608 | 0.336 | 0.353 | 0.519 | 0.568 |
| BAC-CT | 224CP | BT_224 | SKAT | N | 13. | 72 | .120 | 20. | 31 | .16 | 3. | 40 | .83 | 30. | 77 | .70 |
| BAC-CT | 224CP | BT_224 | SKAT | Freq | 205 | | 0.24 | 67 | | 0.53 | 126 | | 0.16 | 177 | | 0.39 |
| BAC-CT | 225CP | BT_225 | ANDRO | Effect | 0.032 | | 0.110 | 0.420 | | −0.100 | −0.034 | | −0.066 | 0.160 | | 0.150 |
| BAC-CT | 225CP | BT_225 | ANDRO | Effect/ | 0.025 | | 0.087 | 0.509 | | −0.120 | −0.032 | | −0.063 | 0.153 | | 0.143 |
| | | | | RMSE | | | | | | | | | | | | |
| BAC-CT | 225CP | BT_225 | ANDRO | Pvalue | 0.859 | | 0.637 | 0.003 | | 0.621 | 0.898 | | 0.831 | 0.183 | | 0.389 |
| BAC-CT | 225CP | BT_225 | ANDRO | LSM | −0.73< | −0.58 | >−0.66 | −1.32< | −1 | <−0.47 | −0.012> | −0.11 | <−0.079 | −0.87< | −0.57 | <−0.56 |
| BAC-CT | 225CP | BT_225 | ANDRO | Contrasts | 0.702 | 0.679 | 0.858 | 0.177 | 0.040 | 0.003 | 0.85 [symbol] | 0.867 | 0.899 | 0.172 | 0.950 | 0.16 [symbol] |
| BAC-CT | 225CP | BT_225 | ANDRO | N | 14. | 73 | .120 | 20. | 32 | .17 | 4. | 45 | .89 | 30. | 76 | .70 |
| BAC-CT | 225CP | BT_225 | ANDRO | Freq | 207 | | 0.24 | 69 | | 0.52 | 138 | | 0.19 | 176 | | 0.39 |
| BAC-CT | 225CP | BT_225 | SKAT | Effect | −0.220 | | −0.070 | −0.100 | | 0.130 | 0.350 | | 0.400 | 0.062 | | 0.180 |
| BAC-CT | 225CP | BT_225 | SKAT | Effect/ | −0.164 | | −0.060 | −0.098 | | 0.123 | 0.284 | | 0.331 | 0.060 | | 0.168 |
| | | | | RMSE | | | | | | | | | | | | |
| BAC-CT | 225CP | BT_225 | SKAT | Pvalue | 0.210 | | 0.751 | 0.661 | | 0.618 | 0.338 | | 0.323 | 0.586 | | 0.288 |
| BAC-CT | 225CP | BT_225 | SKAT | LSM | 4.66> | 4.37 | >4.22 | 4.19< | 4.22 | >3.99 | 3.04< | 3.79 | >3.73 | 3.03< | 3.27 | >3.16 |
| BAC-CT | 225CP | BT_225 | SKAT | Contrasts | 0.421 | 0.406 | 0.210 | 0.830 | 0.475 | 0. [symbol] | 0.306 | 0.808 | 0.336 | 0.282 | 0.513 | 0.586 |
| BAC-CT | 225CP | BT_225 | SKAT | N | 13. | 72 | .120 | 20. | 31 | .16 | 3. | 40 | .83 | 30. | 77 | .70 |
| BAC-CT | 225CP | BT_225 | SKAT | Freq | 205 | | 0.24 | 67 | | 0.53 | 126 | | 0.16 | 177 | | 0.39 |
| BAC-CT | 226CP | BT_225 | ANDRO | Effect | −0.032 | | 0.110 | −0.420 | | −0.100 | 0.034 | | −0.066 | −0.150 | | 0.150 |
| BAC-CT | 226CP | BT_226 | ANDRO | Effect/ | −0.025 | | 0.087 | −0.509 | | −0.120 | 0.032 | | −0.063 | −0.152 | | 0.144 |
| | | | | RMSE | | | | | | | | | | | | |
| BAC-CT | 226CP | BT_226 | ANDRO | Pvalue | 0.859 | | 0.637 | 0.003 | | 0.621 | 0.899 | | 0.831 | 0.181 | | 0.363 |
| BAC-CT | 226CP | BT_226 | ANDRO | LSM | −0.66< | −0.58 | >−0.73 | −0.47> | −1 | >−1.32 | −0.079> | −0.11 | <−0.012 | −0.056> | −0.57 | >−0.87 |
| BAC-CT | 226CP | BT_226 | ANDRO | Contrasts | 0. [symbol] | 0.702 | 0.859 | 0.040 | 0.177 | 0.003 | 0.867 | 0.856 | 0.888 | 0.964 | 0.168 | 0.161 |
| BAC-CT | 226CP | BT_226 | ANDRO | N | 120. | 73 | .14 | 17. | 32 | .20 | 69. | 45 | .4 | 70. | 75 | .31 |
| BAC-CT | 226CP | BT_226 | ANDRO | Freq | 207 | | 0.76 | 69 | | 0.48 | 138 | | 0.81 | 176 | | 0.61 |
| BAC-CT | 226CP | BT_226 | SKAT | Effect | 0.220 | | −0.070 | 0.100 | | 0.130 | −0.350 | | 0.400 | −0.066 | | 0.190 |
| BAC-CT | 226CP | BT_226 | SKAT | Effect/ | 0.184 | | −0.060 | 0.098 | | 0.123 | −0.284 | | 0.331 | −0.064 | | 0.178 |
| | | | | RMSE | | | | | | | | | | | | |
| BAC-CT | 226CP | BT_226 | SKAT | Pvalue | 0.210 | | 0.751 | 0.561 | | 0.818 | 0.336 | | 0.323 | 0.556 | | 0.259 |
| BAC-CT | 226CP | BT_226 | SKAT | LSM | 4.22< | 4.37 | <4.68 | 3.99< | 4.22 | >4.19 | 3.73< | 3.79 | >3.04 | 3.16< | 3.28 | >3.02 |
| BAC-CT | 226CP | BT_226 | SKAT | Contrasts | 0.40 [symbol] | 0.421 | 0.210 | 0.476 | 0.930 | 0.561 | 0.808 | 0.308 | 0.338 | 0.480 | 0.258 | 0.556 |
| BAC-CT | 226CP | BT_226 | SKAT | N | 120. | 72 | .13 | 16. | 31 | .20 | 83. | 40 | .3 | 70. | 76 | .31 |
| BAC-CT | 226CP | BT_226 | SKAT | Freq | 205 | | 0.76 | 57 | | 0.47 | 126 | | 0.82 | 177 | | 0.61 |

[symbol]

indicates data missing or illegible when filed

### Example 3

The following tables show single marker and multiple marker analysis for the different combinations of markers.

| Cells (genotype x marker x breed combinations) with 5% or less animals tested were excluded from the analysis. | | | | | | | | | |
|---|---|---|---|---|---|---|---|---|---|
| Y | Label | Duroc | Hampshire | LW_Duroc | Landrace | LargeWhite | Pietrain | SireLine | Yorkshire |
| SKAT | 157CP | 4.47 | 12.31 | 0.10 | 1.58 | 0.64 | 6.43 | 0.48 | 10.15 |
| SKAT | 221CP | 3.68 | 0.12 | 0.44 | 1.24 | 1.47 | 6.43 | 1.17 | 1.03 |
| SKAT | 222CP | 16.54 | 0.11 | 0.54 | 0.12 | 1.53 | 0.47 | 1.30 | 1.97 |
| SKAT | 173CP | 9.60 | 1.79 | 0.12 | 3.30 | 1.07 | 6.83 | 3.75 | 3.01 |
| SKAT | 227CP | 1.16 | 0.03 | 0.78 | 0.38 | 1.18 | 0.92 | 0.10 | 0.62 |
| SKAT | 238CP | 0.00 | 1.52 | 0.55 | 0.11 | 0.27 | 7.70 | 0.00 | 0.35 |
| SKAT | 239CP | 0.02 | 0.38 | 0.26 | 0.08 | 1.50 | 3.02 | 3.38 | 2.27 |
| SKAT | 240CP | 1.54 | 0.14 | 0.50 | 0.24 | 0.28 | 1.44 | 0.32 | 0.29 |
| SKAT | 152CP | 6.12 | 0.03 | 0.28 | 0.03 | 0.47 | 4.64 | 0.00 | 0.80 |
| SKAT | 153CP | 14.34 | 0.21 | 1.34 | 0.05 | 0.08 | 1.26 | 1.47 | 0.01 |
| SKAT | 158CP | 13.08 | 0.21 | 1.29 | 0.23 | 0.06 | 1.48 | 1.22 | 0.05 |
| SKAT | 193CP | 10.82 | 0.32 | 1.26 | 0.25 | 0.13 | 2.55 | 1.42 | 0.13 |
| SKAT | 156CP | 6.69 | 0.00 | 0.95 | 6.97 | 0.90 | 0.68 | 0.00 | 0.04 |
| SKAT | 161CP | 24.20 | 3.33 | 0.02 | 3.83 | 0.42 | 0.00 | 0.00 | 0.00 |
| SKAT | 140CP | 1.28 | 0.32 | 1.69 | 1.15 | 1.11 | 2.69 | 0.71 | 1.21 |
| SKAT | 141CP | 6.49 | 2.18 | 0.08 | 0.85 | 1.13 | 0.00 | 0.35 | 0.22 |
| SKAT | 162CP | 3.89 | 9.51 | 1.22 | 0.08 | 0.68 | 0.42 | 0.03 | 1.20 |
| SKAT | 171CP | 12.17 | 5.92 | 0.73 | 2.93 | 1.12 | 1.23 | 0.66 | 2.31 |
| SKAT | 223CP | 0.04 | 0.33 | 1.99 | 0.79 | 0.94 | 0.56 | 0.05 | 0.67 |
| SKAT | 224CP | 0.04 | 0.39 | 1.63 | 0.72 | 0.95 | 0.83 | 0.05 | 0.56 |
| SKAT | 225CP | 0.04 | 0.27 | 1.70 | 0.75 | 0.95 | 0.85 | 0.05 | 0.69 |
| SKAT | 226CP | 0.04 | 0.27 | 1.70 | 0.85 | 0.95 | 0.85 | 0.05 | 0.78 |
| ANDRO | 157CP | 0.03 | 1.66 | 0.05 | 1.22 | 0.46 | 0.37 | 0.04 | 0.46 |
| ANDRO | 221CP | 0.24 | 0.29 | 0.47 | 0.52 | 1.46 | 7.61 | 4.41 | 1.21 |
| ANDRO | 222CP | 2.21 | 0.40 | 0.21 | 5.51 | 0.24 | 6.01 | 0.73 | 0.10 |
| ANDRO | 173CP | 1.67 | 1.36 | 0.20 | 1.02 | 1.07 | 11.69 | 5.15 | 1.62 |
| ANDRO | 227CP | 2.02 | 0.54 | 2.18 | 0.01 | 0.10 | 1.26 | 1.30 | 0.72 |
| ANDRO | 238CP | 0.00 | 2.85 | 0.06 | 0.01 | 0.23 | 0.15 | 0.00 | 0.01 |
| ANDRO | 239CP | 1.76 | 0.65 | 0.20 | 0.88 | 1.22 | 2.88 | 0.92 | 0.11 |
| ANDRO | 240CP | 1.82 | 1.88 | 1.38 | 0.02 | 0.05 | 0.95 | 2.14 | 0.59 |
| ANDRO | 152CP | 0.00 | 2.00 | 0.06 | 1.08 | 1.11 | 0.01 | 0.41 | 1.51 |
| ANDRO | 153CP | 0.20 | 0.82 | 0.74 | 0.46 | 0.18 | 1.73 | 1.18 | 1.42 |
| ANDRO | 158CP | 0.17 | 0.82 | 0.86 | 0.53 | 0.18 | 3.08 | 0.99 | 1.10 |
| ANDRO | 193CP | 0.22 | 0.85 | 0.71 | 0.49 | 0.85 | 3.45 | 0.73 | 5.25 |
| ANDRO | 156CP | 0.05 | 0.00 | 0.82 | 1.33 | 1.26 | 1.58 | 0.00 | 1.79 |
| ANDRO | 161CP | 3.76 | 0.38 | 0.12 | 0.60 | 0.24 | 0.00 | 0.00 | 0.00 |
| ANDRO | 140CP | 5.15 | 0.58 | 0.99 | 1.27 | 1.25 | 3.70 | 0.51 | 0.67 |
| ANDRO | 141CP | 0.46 | 0.81 | 0.00 | 0.09 | 0.02 | 0.00 | 0.14 | 1.00 |
| ANDRO | 162CP | 7.25 | 4.80 | 0.03 | 3.10 | 1.52 | 2.90 | 2.28 | 0.80 |
| ANDRO | 171CP | 2.58 | 4.07 | 0.06 | 1.36 | 0.44 | 2.44 | 0.46 | 0.49 |
| ANDRO | 223CP | 5.30 | 0.32 | 0.93 | 0.04 | 0.34 | 15.91 | 0.02 | 1.16 |
| ANDRO | 224CP | 5.30 | 0.30 | 0.92 | 0.07 | 0.12 | 12.75 | 0.02 | 0.92 |
| ANDRO | 225CP | 5.30 | 0.32 | 0.76 | 0.12 | 0.12 | 12.75 | 0.02 | 1.29 |
| ANDRO | 226CP | 5.30 | 0.32 | 0.76 | 0.10 | 0.12 | 12.75 | 0.02 | 1.31 |

Sequence data

| SNP | Gene | | Annealing | Primer | |
|---|---|---|---|---|---|
| code | Code | Gene Name | Temperature | Name | Primer Sequence (5′-3′) |
| 140CP | SULT1A1 | sulfotransferase 1A1 | 58 | 140CP-F | GTACTTTGCAGAGGCACTGG |
| | | | | | (SEQ ID NO: 1) |
| | | | | 140CP-R | GATTTGGGATAGGTGCTGATC |
| | | | | | (SEQ ID NO: 2) |
| 141CP | SULT1A1 | sulfotransferase 1A1 | 58 | 141CP-F | GTTTTGAGCTGCTGAAAGATACAC |
| | | | | | (SEQ ID NO: 3) |
| | | | | 141CP-R | CTGGTCCAGCAGAGTCTGG |
| | | | | | (SEQ ID NO: 4) |
| 152CP | CYP2E1 | cytochrome P450 2E1 | Touch-down | 152/3CP-F | TGACCCCAAGGATATCGAC |
| | | | | | (SEQ ID NO: 5) |
| | | | | 152/3CP-R | GCACATCTCCCTCACACTTGT |
| | | | | | (SEQ ID NO: 6) |
| 153CP | CYP2E1 | cytochrome P450 2E1 | Touch-down | 152/3CP-F | TGACCCCAAGGATATCGAC |
| | | | | | (SEQ ID NO: 7) |
| | | | | 152/3CP-R | GCACATCTCCCTCACACTTGT |
| | | | | | (SEQ ID NO: 8) |
| 156CP | CYTB5 | cytochrome B5 | 58 | 156CP-F | GACTCCCACTCTGTTCCGC |
| | | | | | (SEQ ID NO: 9) |
| | | | | 156CP-R | CCAGGGTGTAATACTTCACGG |
| | | | | | (SEQ ID NO: 10) |
| 157CP | 3αHSD | 3 alpha hydroxysteroid | Touch-down | 157CP-F | CCCAAGAGTGAAGCTCTGGA |
| | | dehydrogenase | | | (SEQ ID NO: 11) |
| | | | | 157CP-R | CTCTCTTCACGGTGCCATCT |
| | | | | | (SEQ ID NO: 12) |
| 158CP | CYP2E1 | cytochrome P450 2E1 | 58 | 158CP-F | CAAGTGTGAGGGAGATGTGC |
| | | | | | (SEQ ID NO: 13) |
| | | | | 158CP-R | TTGATTTCCTATGGAGCCC |
| | | | | | (SEQ ID NO: 14) |
| 161CP | CYB5 | cytochrome B5 | 58 | 161CP-F | TGAGCCATGGTGTTCTAGAGA |
| | | | | | (SEQ ID NO: 15) |
| | | | | 161CP-R | CAGGCAGAGGGTGATATACGT |
| | | | | | (SEQ ID NO: 16) |
| 162CP | SULT1A1 | sulfotransferase 1A1 | 58 | 162CP-F | ACTGTTGGGATGTTGTACAGG |
| | | | | | (SEQ ID NO: 17) |
| | | | | 162CP-R | AGTACTTGATGAGAGGGACCC |
| | | | | | (SEQ ID NO: 18) |
| 171CP | SULT1A1 | sulfotransferase 1A1 | 58 | 171CP-F | AAAAGCTTGGTCAGAGAAAGC |
| | | | | | (SEQ ID NO: 19) |
| | | | | 171CP-R | AGTTTTGTGGCAGCTCTCC |
| | | | | | (SEQ ID NO: 20) |
| 173CP | CYP17A1 | cytochrome P450 17A1 | 56 | 173CP-F | CGGGAAATCCTTGAAAACC |
| | | | | | (SEQ ID NO: 21) |
| | | | | 173CP-R | AGTGTCCAAAATGAACCCAA |
| | | | | | (SEQ ID NO: 22) |
| 193CP | CYP2E1 | cytochrome P450 2E1 | 56 | 193CP-F | TTTGGTAGTAATCAGAGATGAACTT |
| | | | | | (SEQ ID NO: 23) |
| | | | | 193CP-R | TGAATTTCACTCCACTTTGG |
| | | | | | (SEQ ID NO: 24) |
| 221CP | 3βHSD | 3 alpha hydroxysteroid | 58 | 221CP-F | AGTGTTTTCTGGTTCCTGGC |
| | | dehydrogenase | | | (SEQ ID NO: 25) |
| | | | | 221CP-R | CTCTGACCCAGAAACCCTC |
| | | | | | (SEQ ID NO: 26) |
| 222CP | 3βHSD | 3 alpha hydroxysteroid | 58 | 222CP-F | ACGACACACCTCCCCAAAG |
| | | dehydrogenase | | | (SEQ ID NO: 27) |
| | | | | 222CP-R | GCCAGCCAGTACCTCAGAGA |
| | | | | | (SEQ ID NO: 28) |
| 223CP | BAC-CT | BAC end sequence | 58 | 223CP-F | TCAGGTTGCTGCTATGGTG |
| | | CT171681 | | | (SEQ ID NO: 29) |
| | | | | 223CP-R | AAGTGGCATCTTCCTCTGAA |
| | | | | | (SEQ ID NO: 30) |
| 224CP | BAC-CT | BAC end sequence | 58 | 224CP-F | CTCTTAGGTCTCCCCCTCG |
| | | CT171681 | | | (SEQ ID NO: 31) |
| | | | | 224CP-R | AACTTAGGGCTCAGACAGGC |
| | | | | | (SEQ ID NO: 32) |
| 225CP | BAC-CT | BAC end sequence | 58 | 225/6CP-F | CCTTTTAACCTGTTTCACCCT |
| | | CT171681 | | | (SEQ ID NO: 33) |
| | | | | 225/6CP-R | GGCAGGTAGGCACAGAGAC |
| | | | | | (SEQ ID NO: 34) |
| 226CP | BAC-CT | BAC end sequence | 58 | 225/6CP-F | CCTTTTAACCTGTTTCACCCT |
| | | CT171681 | | | (SEQ ID NO: 35) |
| | | | | 225/6CP-R | GGCAGGTAGGCACAGAGAC |
| | | | | | (SEQ ID NO: 36) |
| 238CP | CYP2A | cytochrome P450 2A6 | 58 | 238CP-F | ACTGCTGTGGTCCCTGTGT |
| | | | | | (SEQ ID NO: 37) |
| | | | | 238CP-R | TTCTTCCTCCAGTGATGGG |
| | | | | | (SEQ ID NO: 38) |
| 239CP | CYP2A | cytochrome P450 2A6 | Touch-down | 239CP-F | GTCCTCAGCACACCCACAC |
| | | | | | (SEQ ID NO: 39) |
| | | | | 239CP-R | CAGGTCCTTAGGGAAGCCT |
| | | | | | (SEQ ID NO: 40) |
| 240CP | CYP2A | cytochrome P450 2A6 | Touch-down | 239CP-F | GTCCTCAGCACACCCACAC |
| | | | | | (SEQ ID NO: 41) |
| | | | | 239CP-R | CAGGTCCTTAGGGAAGCCT |
| | | | | | (SEQ ID NO: 42) |

SEQUENCE OF AMPLICON

140CP
(SEQ ID NO: 43)
GTACTTTGCAGAGGCACTGGGGCCACTGGAGAGTTTCCAAGCTTGGCC
CGATGA(C/T)GTGCTGATCAGCACCTATCCCAAATC
141CP
(SEQ ID NO: 44)
GTTTTGAGCTGCTGAAAGATACACCAGCCCCACGGCTCCTCAAGACAC
ACTTGCCCCTG(A/G)CCCTGCTACCCCAGACTCTGCTGGACCAG
152CP
(SEQ ID NO: 45)
TGACCCCAAGGATATCGACCTCAGCCCCAT(C/T)RCGATTGGGTTTG
CCAAGATTCCCCCCCATTACAAACTCTGTGTCATTCCCCGCTCACAAG
TGTGAGGGAGATGTGC
153CP
(SEQ ID NO: 46)
TGACCCCAAGGATATCGACCTCAGCCCCATY(A/G)CGATTGGGTTTG
CCAAGATTCCCCCCCATTACAAACTCTGTGTCATTCCCCGCTCACAAG
TGTGAGGGAGATGTGC
156CP
(SEQ ID NO: 47)
GACTCCCACTCTGTTCCGCTCATCTCTGCCGCTGTCAGCAGGGCCTGA
GGTTCGCCGC(G/T)TTACGAAATGGCCGAACAGTCCGACAAAGCCGT
GAAGTATTACACCCTGG
157CP
(SEQ ID NO: 48)
CCCAAGAGTGAAGCTCTGGAGGCCACCAAATATGCCATAGAAGTTGGG
TTCCGTCA(C/T)ATCGATAGTGCTTATTTATACCAAAATGAAGAGCA
GGTTGGACAGGCCATTCGAAGCAAGATTGCAGATGGCACCGTGAAGAG
AG
158CP
(SEQ ID NO: 49)
CAAGTGTGAGGGAGATGTGCTC(G/T)AAAGGCCCTGGTTCCTTGATG
CTGACCTGGAGGCCTCCTGTCCCCAGTGTCCCCACAGGGAGCGCAGCC
CGGGCTCCATAGGAAATCAA
161CP
(SEQ ID NO: 50)
TGAGCCATGGTGTTCTAGAGAAATAACTAAAACACATTGGAAAGGAAT
TTTTCTAAATAACAGAGCATC(A/G)TAGATTTTTATAATCAATGACG
TATATCACCCTCTGCCTG
162CP
(SEQ ID NO: 51)
ACTGTTGGGATGTTGTACAGGGGAGGAGAG(C/T)GAGCTCGCAGCAT
GGAGCCGGTCCAGGACACCTACCGCCCGCCACTGGAGTACGTGAAGGG
GGTCCCTCTCATCAAGTACT
171CP
(SEQ ID NO: 52)
AAAAGCTTGGTCAGAGAAAGCTGGGGGCTGAGACAGGCAGGCCCTGGA
(A/G)TAGTGATTTTTTTCAAGTGCACACTGGAGCACCCCCGGAGAGC
TGCCACAAAACT
173CP
(SEQ ID NO: 53)
CGGGAAATCCTTGAAAACCGTAAGGTAGGTGGTGATGAAGCAGGAGAG
ATGACGAATTAGGTTGAAAGTGTCCTGA(A/G)AGCAGGCTTGGGTTC
ATTTTGGACACT
193CP
(SEQ ID NO: 54)
TTTGGTAGTAATCAGAGATGAACTTTTTTGAAATTTGTCAACTCTTTT
CCTTTCTCTTTTCCTCCCCCA(C/T)TGAATTTGCCAGTTGATTTCCC
AAAGTGGAGTGAAATTCA
221CP
(SEQ ID NO: 55)
AGTGTTTTCTGGTTCCTGGCAAGTATTTCTCGG(C/T)GCCCAGGTTT
AGCAATGGCTGGATGGAGCTGCCTTGTGACAGGAGGAGGAGGGTTTCT
GGGTCAGAG
222CP
(SEQ ID NO: 56)
ACGACACACCTCCCCAAAGCTACGATGACCTCAATTACACGTTGGGCA
AGGA(A/G)TGGGGCTTCTGCCTTGATTCCAGAAGGAGCCTTCCGCCC
TCTCTGAGGTACTGGCTGGC
223CP
(SEQ ID NO: 57)
TCAGGTTGCTGCTATGGTGCAGGTTTGATCCC(C/T)AGTCTGGGAAT
TTCTGCATGCCATGGGCATGGCCAAAAATAAATAAATAAAATAAAAAG
AGTGTGACTTCAGAGGAAGATGCCACTT
224CP
(SEQ ID NO: 58)
CTCTTAGGTCTCCCCCTCGCTTTCTCCAAGACAATCTGTGAATCCAGG
TGTCATCATACAT(A/G)CAGCCACATGGGGGCAGTGTGGGCCTGTCT
GAGCCCTAAGTT
225CP
(SEQ ID NO: 59)
CCTTTTAACCTGTTTCACCCTCCATCACCGGAGGCCAGGAGAAGC
(A/C)TGGGCTGAGCCCCTTCCTCCCACAGCTCTGCCTCTCCRCAGCT
TTCTATGTCTCTGTGCCTACCTGCC
226CP
(SEQ ID NO: 60)
CCTTTTAACCTGTTTCACCCTCCATCACCGGAGGCCAGGAGAAGCMTG
GGCTGAGCCCCTTCCTCCCACAGCTCTGCCTCTCC(A/G)CAGCTTTC
TATGTCTCTGTGCCTACCTGCC
238CP
(SEQ ID NO: 61)
ACTGCTGTGGTCCCTGTGTCCAATGCTCACACCAGTCTCCGCACCCGC
CCGCTGCTGGACTTGATCTCTGCTTGGCCCCCAGCAT(A/G)GGCCAG
GCCCATCACTGGAGGAAGAA
239CP
(SEQ ID NO: 62)
GTCCTCAGCACACCCACACGTCAAATG(A/G)GAAGCATTGATCCTAA
CAGTGATGCTGCTGCTGCTGCTGCTGATGGAAACGGTCCCATCAACCC
AGCAGGCTTCCCTAAGGACCTG
240CP
(SEQ ID NO: 63)
GTCCTCAGCACACCCACACGTCAAATGAGAAGCATTGATCCTAACAGT
GATGCTGCTGCTGCTGCTGCTGATGGAAA(C/T)GGTCCCATCAACCC
AGCAGGCTTCCCTAAGGACCTG

The SNP of interest id indicated in brackets.

NOVEL SEQUENCE

CYP2A Gene Sequence
(SEQ ID NO: 64)
ACTATAGGGCACGCGTGGTCGACGGCCCGGGCTGGTCCTACCTGATGC
CAAGGGCGGTGCCTACTGCTGTGGTCCCTGTGTCCAATGCTCACACCA
GTCTCCGCACCCGCCCGCTGCTGGACTTGATCTCTGCTTGGCCCCCAG
CAT[G/A]¹⁵⁹⁶GGCCAGGCCCATCACTGGAGGAAGAACAAGGAGAGA
GGGTTCAGATCCCAGCTCCTAAGCTTACCTGCTCCCTGCGTGACCTCC
AGCAAGTGGCTTTAGAGAGGCTCCTCTTCTCAACTGCAAAATGAAGCC
GATGATGGACCTGCCCTGTTGTCATAAGGATTCAATAAGGCCACGCAT
ATGTAGACTCAGTCCTCACAGGCAGTGCTTCCCGGGGTAACCATCGTT
CTAAAGGAAGCACATGGGGTGGGGAGAGGACAGCAGGGCCACCCCCCT
CCTTTCTGCACCCACTTCCAGCATCCCAGGGACCCCTCAGTTCCTGAC
ACAGGAGTCCACCCACTTCTCTCTTAACATAGCTCCCTCTGCCTGCAA
AGAGCAGCCCCGACAAACCGGGAATCACCCCTAAAGGGGACTTGACAC
CCCCTCAAATACAACCTTCTCTTCCCAAATGCTCCCTTTCCATGGTGG
GAAAACTCGACCCCAGAAGGCGAGTGCAAAGCAGGAGAGACAGGGGGC
ACACGTGTGCCCCTTGCCCACTCTCTGTCTTCTGTCCTCAGCACACCC
ACACGTCAAATG[G/A]¹⁰¹⁹GAAGCATTGATCCTAACAGTGATGCTG
CTGCTGCTGCTGCTGATGGAAA[C/T]⁹⁶⁸GGTCCCATCAACCCAGCA
GGCTTCCCTAAGGACCTGGGGAGGGAAGGAGCAGGGCCCTCTGTGAGT
TCTGATCCTTGACACAGTTGGGATTTTTCAGTATCAGGCTGGCGGTTA
GTCCTGTTCCCCAAGCCCTGGCCAGTCCCTCTGCCAGCTGAAACCATG
AGTTATTCTTCTCCAGTTCTGTCAAAGGTTGGACAGAAATGCAGCTCT
GGTCTTCTACCGCTTACCCAACCAGACCTGGGCAATTCTGTGACACCC
TCCTGGCCTCGCTTTGAGGTTCCAATGACAATTCCGGGGATCAAGGGG
CGGCACTGTGTCCAAAATAATAGCAGGTCAATAACTGGGGTCAGGTGC
TAACGCCCTGATCCAGCTGAACTCTCTTCCCAGCAACCCCTCATCCAC
AGCTCTGGTCCTTTCTCACTGCAGCACCCTCAAATCTATTCTCTAGAA
TCCCCTCCCCAGGCATAAGACCCTTGAATCTACCTCCGTTCTCACTGA
AAGATCCCCAAATCTGCAGCCACACATCCTGCCTCATTCCAATACCCT
TAAATCCAGGTCTTTGAATTCTTCTTTCCTGAGACCTCAAAATCCACA
ACTTTGGAGTCAGTTCTCCCTCTGAGACTCCCAATCCAAAGTTCAGGG
GTTCACCCCAAAACAACTAGTCCAAAGTCTTCAGTTCTGTAACTTATC
TACTGCCCCCTCCAAAGTCCAAAGCCAAGACTAGCCCCTTCTGGGGGA
CCACAAATTCCATCTTAGGGCACACTCCCTGTTAATCTGAACTGGGGT
CCCCCTCCTCCTTCCTGGCTGGCTACGTCCCAAGCTAGGCGGGGAGCA
TCACAGGGGGTGTAGTTGGGAGGTGAAATGAGACAGTTATATAATCAG
GACCAAAGCCTGCCCTTCTCTCCCAGGCGGTATAAAAGCACCCATCCC
AACCCATCACCAACTGACCGTCCCTCGCAGTGCCACC [symbol] CTGGCCTC
AGGCTTGCTTCTCGTGGCTCTGCTGACCTGCCTGACCATAATGGTCTT
GATGTCCGTCTGGCGCCAGAGGAAGCTCCAGGGGAAACTGCCCCCCGG
ACCCACCCCGCTGCCCTTCATCGGGAACTACCTGCAGCTGAACACGGA
GCAGATGTACAACTCCCTCATGAAGATCAGCCAGCGCTATGGCCCTGT
GTTCACCGTCCACCTGGGGCCCCGGCGGATAGTGGTGCTGTGTGGATA
CGACGCGGTGAAGGAGGCCCTGGTGGACCAGGCTGAGGAATTCAGCGG
GCGAGGCGAGCAGGCCACTTTCGACTGGCTCTTCAAAGGCTATGGCGT
GGCCTTCAGCAACGGCGAGCGTGCCAAGCAGCTCCGGCGCTTCTCCAT
CACCACGCTGCGGGACTTCGGCGTGGGCAAGCGGGGTATCGAGGAGCG
CATCCAGGAGGAGGCGGGCCACCTCATCGAGGCCTTCCGGGGCACGCG
CGGCGCGTTCATCGACCCCACCTACTTCCTCAGCCGAACGGTTTCCAA
TGTCATCAGCTCCATTGTCTTCGGAGACCGCTTTGACTATGAGGACAA
AGAGTTCCTCGCACTGCTGCGGATGATGCTGGGAAGCTTTCAGTTCAC
AGCTACCTCTACCGGACAGCTCTATGAGATGTTCTACTCGGTGATGAA
ACACCTGCCAGGGCCGCAGCAACAGGCATTTAAGGACCTGCAGGGGCT
GGAGGACTTCATAGCCAGGAAGGTGGAACACAACCAGCGCACGCTGGA
TCCCAACTCCCCGCGAGACTTCATCGACTCCTTCCTCATCCGCATGCA
GGAGGAGAAGAAGAATCCTGACACCGAGTTCTATTGGAAGAACCTGGT
TCTGACCACACTGAACCTCTTCTTCGCGGGCACCGAGACGGTCAGCAC
AACGATGCGCTACGGCTTCCTGCTGCTCATGAAGAAACCGGATGTGGA
GGCCAAAGTCCACGAGGAGATTGACCGCGTGATCGGCAGGAACCGCCA
GGCCAAGTTCGAGGACCGGGCCAAGATGCCCTACACGGAGGCCGTGAT
CCACGAGATCCAGAGATTCOGAGACATGATCCCCATGGGCCTGGCCCG
AAGAGTCACCAAGGATACCAAGTTTCGGGACTTCCTCCTCCCCAAGGG
CACTGAGGTGTTCCCTATGCTGGGCTCTGTGCTGAGAGACCCCAAGTT
CTTCTCCAACCCCCGAGGCTTCAACCCCCAGCACTTCCTGGATGAGAA
CGGGCAGTTTAAGAAGAATGATGCTTTTGTGCCCTTCTCCATCGGAAA
GCGGTACTGTTTCGGAGAAGGTCTGGCTAGAATGGAGCTCTTCCTCTT
CCTCACCAACATCCTGCAGAACTTCCACCTCAAGTCTCCGCAGCTGCC
CCAGGACATCGACGTGTCCCCCAAACACGTGGGCTTCGCCACCATCCC
CCCGACCTACACCATGAGCTTCCAGCCCCGCTGAGCCCGGGCTGTGCC
AGGGCAGGGCTCGGGGGAGGAGCGAGGGGGCGGGGGCGGGGAGGGGGC
GGGGCTAACGCCAGGGGATGGGGGACCCAGGGGGAAGGGTGGAGAGGA
GAGGAGGAAGGAACAGAACGGAGGAGCTGTTCACTTTACTAGAAATGG
AGTCTTCCGAGGCCCGGCGGGAGGGAAAGAAGACTTTTCTTCTTTTTA
AGACGATGCTTGGAGTAATAACAATAACACGTTTTTTTTCCTAAAAAA
AAAAAAAAAAAAAAAAAA
nb:- Start codon is boxed; sequence starts at
−1743 from start codon (also see GenBank entry
AJ888470)
SNP at position −1596 = 238CP
SNP at position −1019 = 239CP
SNP at position −968 = 240CP
3 αHSD Gene Sequence
(SEQ ID NO: 65)
CGGGAGCTCTGGTG [symbol] GATCCCAAAAGCCAGCGTCTTCGGCTTAACG
ATGGTCACTTCATTCCTGTACTGGGATTTGGTACCTATGCACCTGAAG
AGGTTCCCAAGAGTGAAGCTCTGGAGGCCACCAAATATGCCATAGAAG
TTGGGTTCCGTCA[C/T]ATCGATAGTGCTTATTTATACCAAAATGAA
GAGCAGGTTGGACAGGCCATTCGAAGCAAGATTGCAGATGGCACCGTG
AAGAGAGAAGACATATTCTACACGTCAAAGCTTTGGGCCACTTTCCTT
CGACCAGAGTTGGTCCGACCAGCCTTGGAAAAGTCCCTGAAGAATCTC
CAACTGGACTATGTGGATCTCTATATTATTCATTTTCCAGTGGCTCTG
AAGCCCGGGGAGGAACTTTTGCCAACAGATGAAAACGGAAAAGCACTA
TTTGACACAGTGGATCTCTGTCGCACGTGGGAGGCCTTGGAGAAGTGT
AAGGACGCAGGACTGACCAAGTCCATCGGCGTGTCCAACTTTAACCAC
CAACAGCTGGAGAGGATCCTGAACAAGCCAGGGCTCAAGTACAAGCCC
GTCTGCAACCAGGTGGAATGTCATCCTTACCTCAACCAGAGCAAGCTT
CTGGAGTTTTGCAAGTCCAAGGACATCGTTCTAGTTGCCTATAGTGCA
CTGGGATCCCAAAGAAACTCAAAGTGGGTGGAAGAGAGCAACCCATAT
CTCTTAGAGGATCCAGTCTTAAATGCTATTGCCAAGAAACACAACAGA
AGCCCAGCGCAGGTTGCCCTGCGCTACCAGCTGCAGCGGGGAGTGGTG
GTCCTGGCCAAGAGCTTCAATGAGCAGAGGATCAAAGAGAACTTCCAG
GTTTTTGACTTTGAATTGCCTCCAGAAGATATGAAAACAATCGATGGC
CTCAACCAAAATTTAAGATATTTTAAGTTACTCTTTGCTGTCGATCAC
CCTTACTACCCCTATTCTGAAGAGTACTGAGCGGGAGCTCTCCATCGG
GTGGGCTACCAGAACCTCTTGCTTCTCGGGCTGTGAAGAGGGTTTCTG
TACTTGGTAGAGGTGTTTAAT
nb:- Start codon is boxed; sequence starts at
−14 from start codon
SNP at position 144 = 157CP

As can be seen from the foregoing the invention accomplishes at least all of its objectives. All references cited herein are hereby incorporated in their entirety herein by reference.

What is claimed is:

1. A method of identifying a pig which possesses a genotype indicative of a boar taint, said method comprising: obtaining a nucleic acid sample from said pig, and assaying for the presence of a genotype characterized by a polymorphism or haplotype at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene of the sample, or a polymorphism linked thereto, said genotype being one which has been shown to be significantly associated with a boar taint trait; and associating said pig with said phenotypic trait based upon the genotype present in said pig.

2. The method of claim 1 wherein said step of assaying is selected from the group consisting of restriction fragment length polymorphism (RFLP) analysis, heteroduplex analysis, single strand conformational polymorphism (SSCP), denaturing gradient gel electrophoresis (DGGE), temperature gradient gel electrophoresis (TGGE), allelic PCR, ligase chain reaction, direct sequencing, primer extension, Pyrosequencing, nucleic acid hybridization, micro-array-type detection.

3. The method of claim 1 wherein said amplification includes the steps of: selecting a forward and a reverse primer capable of amplifying a region of an 3αHSD, 3βHSD, CYP17A1, CYP2A, CYP2E1, CYTB5, BAC-CT and/or SULT1A1 nucleotide sequence which contains one or more polymorphic sites.

4. The method of claim 3 wherein said forward and reverse primers for amplifying a region of a 3αHSD nucleotide sequence which contains one or more polymorphic sites are selected from SEQ ID NOs: 11-12 , wherein said forward and reverse primers for amplifying a region of a 3βHSD nucleotide sequence which contains one or more polymorphic sites are selected from SEQ ID NOs: 25-28, wherein said forward and reverse primers for amplifying a region of a CYP17A1 nucleotide sequence which contains one or more polymorphic sites are selected from SEQ ID NOs: 21-22, wherein said forward and reverse primers for amplifying a region of a CYP2A nucleotide sequence which contains one or more polymorphic sites are selected from SEQ ID NOs: 37-42, wherein said forward and reverse primers for amplifying a region of a CYP2E1 nucleotide sequence which contains one or more polymorphic sites are selected from SEQ ID NOs: 5-8, SEQ ID NOs: 13-14, and/or SEQ ID NOs: 23-24, wherein said forward and reverse primers for amplifying a region of a CYTB5 nucleotide sequence which contains one or more polymorphic sites are selected from SEQ ID NOs: 9-10 and/or SEQ ID NOs: 15-16, wherein said forward and reverse primers for amplifying a region of a BAC-CT nucleotide sequence which contains one or more polymorphic sites are selected from SEQ ID NOs: 29-36 and/or wherein said forward and reverse primers for amplifying a region of a SULT1A1 nucleotide sequence which contains one or more polymorphic sites are selected from SEQ ID NOs: 1-4 and/or SEQ ID NOs: 17-20.

5. A method of screening pigs to determine those more likely to exhibit improved boar taint trials comprising: obtaining a biological sample of material from said pig; and assaying for the presence of a genotype in said pig which is associated with favorable boar taint traits said genotype characterized by the following:
a) a polymorphism at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene of the sample, or a polymorphism linked thereto, said polymorphism resulting in one or more restriction sites.

6. The method of claim 5 further comprising the step of amplifying the amount of an 3αHSD, 3βHSD, CYP17A1, CYP2A, CYP2E1, CYTB5, BAC-CT and/or SULT1A1 encoding nucleotide sequence gene or a portion thereof which contains said polymorphism.

7. A method of identifying an pig which possess a desired genotype indicative of a significantly correlated phenotypic trait, the method comprising: obtaining a nucleic acid sample from an pig, said sample comprising a 3αHSD, 3βHSD, CYP17A1, CYP2A, CYP2E1, CYTB5, BAC-CT and/or SULT1A1 gene, digesting the sample with a restriction enzyme that recognizes a polymorphic site, separating the fragments obtained from the digestion, and identifying the presence or absence of restriction site in one allele of the 3αHSD, 3βHSD, CYP17A1, CYP2A, CYP2E1, CYTB5, BAC-CT and/or SULT1A1 gene, wherein the presence of a said allele indicates that the pig possesses a genotype indicative of a significantly associated boar taint trait.

8. A method for selecting pigs for desired boar taint characteristics comprising the steps of: obtaining a nucleic acid sample from an pig, identifying a polymorphism, said polymorphism being a nucleotide at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene characterized by a restriction site, and selecting the pigs which have the nucleotide associated with the desired trait.

9. A method for indirect selection for a polymorphism in an 3αHSD, 3βHSD, CYP17A1, CYP2A, CYP2E1, CYTB5, BAC-CT and/or SULT1A1 gene associated with boar taint comprising: obtaining a nucleic acid sample from an pig, and identifying a polymorphism in an 3αHSD, 3βHSD, CYP17A1, CYP2A, CYP2E1, CYTB5, BAC-CT and/or SULT1A1 gene characterized by a restriction site with a DNA marker known to be associated with the 3αHSD, 3βHSD, CYP17A1, CYP2A, CYP2E1, CYTB5, BAC-CT and/or SULT1A1 gene, said DNA marker further being one which is known to be associated with favorable boar taint traits used to make the indirect identification of the nucleotide substitution, and selecting said pigs based upon the presence of nucleotide substitution.

10. A method of identifying pigs which possess a desired genotype indicative of phenotypic traits, the method comprising: determining an association between an 3αHSD, 3βHSD, CYP17A1, CYP2A, CYP2E1, CYTB5, BAC-CT and/or SULT1A1 H1B genotype and a trait of interest by obtaining a sample of pigs from a line or breed of interest, preparing a nucleic acid sample from each pig in the sample, determining the genotype of the 3αHSD, 3βHSD, CYP17A1, CYP2A, CYP2E1, CYTB5, BAC-CT and/or SULT1A1 gene by screening for a polymorphism, wherein the presence of the polymorphism indicates that the pig possesses a genotype indicative of favorable boar taint trait and calculating the association between the 3αHSD, 3βHSD, CYP17A1, CYP2A, CYP2E1, CYTB5 and/or SULT1A1 genotype and the trait.

11. A method of selecting pigs for breeding, said method comprising: obtaining a nucleic acid sample from said pig; assaying for the presence of a polymorphism at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene of said sample, said polymorphism being one which has previously been shown to be significantly correlated with a boar taint trait; and using the 3αHSD, 3βHSD, CYP17A1, CYP2A, CYP2E1, CYTB5, BAC-CT and/or SULT1A1 genotype as part of a selection model based on the estimated value of the effect of the marker genotype, and thereafter selecting pigs on the basis of this estimated value for use in breeding.

12. A method of segregating pigs in order to provide uniformity at slaughter comprising: obtaining a nucleic acid sample from said pig; and assaying for the presence of a polymorphism at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene of said sample, said polymorphism being one which is associated with boar taint traits, segregating said pigs based upon the polymorphism present in said pig.

13. A method of screening pigs to determine those more likely to produce desired boar taint traits comprising: obtaining a sample of genetic material from said pig; and assaying for the presence of a genotype in said pig which is associated with boar taint, said genotype characterized by the following:
a) a polymorphism at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position 1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene.

14. The method of claim 13 wherein said polymorphism results in an amino acid change of an 3αHSD, 3βHSD, CYP17A1, CYP2A, CYP2E1, CYTB5, BAC-CT and/or SULT1A1 gene or its equivalent as determined by a BLAST comparison.

15. The method of claim 13 wherein said polymorphisms are located in the 3αHSD, 3βHSD, CYP17A1, CYP2A, CYP2E1, CYB5, BAC-CT and/or SULT1A1 genes.

16. The method of claim 13 wherein said genotype is a restriction site polymorphism.

17. The method of claim 13 wherein said step of assaying is selected from the group consisting of: restriction fragment length polymorphism (RFLP) analysis, minisequencing, MALD-TOF, SINE, heteroduplex analysis, single strand conformational polymorphism (SSCP), denaturing gradient gel electrophoresis (DGGE) and temperature gradient gel electrophoresis (TGGE).

18. The method of claim 13 further comprising the step of amplifying the amount of an 3αHSD, 3βHSD, CYP17A1, CYP2A, CYP2E1, CYTB5, BAC-CT and/or SULT1A1 nucleotide sequence or a portion thereof which contains said polymorphism.

19. The method of claim 18 wherein said amplification includes the steps of selecting a forward and a reverse primer capable of amplifying a region of an 3αHSD, 3βHSD, CYP17A1, CYP2A, CYP2E1, CYTB5, BAC-CT and/or SULT1A1 nucleotide sequence which contains one or more polymorphic sites.

20. The method of claim 18 wherein said forward and reverse primers for amplifying a region of a 3αHSD nucleotide sequence which contains one or more polymorphic sites are selected from SEQ ID NOs: 11-12 , wherein said forward and reverse primers for amplifying a region of a 3βHSD nucleotide sequence which contains one or more polymorphic sites are selected from SEQ ID NOs: 25-28, wherein said forward and reverse primers for amplifying a region of a CYP17A1 nucleotide sequence which contains one or more polymorphic sites are selected from SEQ ID NOs: 21-22, wherein said forward and reverse primers for amplifying a region of a CYP2A nucleotide sequence which contains one or more polymorphic sites are selected from SEQ ID NOs: 37-42, wherein said forward and reverse primers for amplifying a region of a CYP2E1 nucleotide sequence which contains one or more polymorphic sites are selected from SEQ ID NOs: 5-8, SEQ ID NOs: 13-14, and/or SEQ ID NOs: 23-24, wherein said forward and reverse primers for amplifying a region of a CYTB5 nucleotide sequence which contains one or more polymorphic sites are selected from SEQ ID NOs: 9-10 and/or SEQ ID NOs: 15-16, wherein said forward and reverse primers for amplifying a region of a BAC-CT nucleotide sequence which contains one or more polymorphic sites are selected from SEQ ID NOs: 29-36 and/or wherein said forward and reverse primers for amplifying a region of a SULT1A1 nucleotide sequence which contains one or more polymorphic sites are selected from SEQ ID NOs: 1-4 and/or SEQ ID NOs: 17-20.

21. An isolated nucleotide sequence or allele which encodes upon expression an 3αHSD, 3βHSD, CYP17A1, CYP2A, CYP2E1, CYTB5, BAC-CT and/or SULT1A1 protein, said nucleotide sequence comprising a polymorphism at position 144 relative to the first nucleotide of the start codon of a 3αHSD gene, position −15 relative to the first nucleotide of the start codon of a 3βHSD gene, position 830 relative to the first nucleotide of the start codon of a 3βHSD gene, in intron 4 of a CYP17A1 gene, position −1596 relative to the first nucleotide of the start codon of a CYP2A gene, position −1019 relative to the first nucleotide of the start codon of a CYP2A gene, position −968 relative to the first nucleotide of the start codon of a CYP2A gene, position 1422 relative to the first nucleotide of the start codon of a CYP2E1 gene, position 1423 relative to the first nucleotide of the start codon of a CYP2E1 gene, position 1502 relative to the first nucleotide of the start codon of a CYP2E1 gene, position 2412 relative to Genbank accession number AJ697882 (CYP2E1 gene), position −8 relative to the first nucleotide of the start codon of a CYTB5 gene, position 1500 relative to the first nucleotide of the start codon of a CYTB5 gene, position 166 relative to the first nucleotide of the start codon of a BAC-CT gene, position 523 relative to the first nucleotide of the start codon of a BAC-CT gene, position 707 relative to the first nucleotide of the start codon of a BAC-CT gene, position 745 relative to the first nucleotide of the start codon of a BAC-CT gene, position −12 relative to the first nucleotide of the start codon of a SULT1A1 gene, position 120 relative to the first nucleotide of the start codon of a SULT1A1 gene, position 334 relative to the first nucleotide of the start codon of a SULT1A1 gene and/or in intron 1 of the SULT1A1 gene.

22. An isolated 3αHSD, 3βHSD, CYP17A1, CYP2A, CYP2E1, CYTB5, BAC-CT and/or SULT1A1 protein according to claim 21.

23. A method of identifying a polymorphism correlated with desired boar taint traits comprising the steps of: obtaining a sample of genetic material from a pig, said sample comprising a 3αHSD, 3βHSD, CYP17A1, CYP2A, CYP2E1, CYTB5, BAC-CT and/or SULT1A1 gene with a sequence set forth in the Examples herein; assaying for said 3αHSD, 3βHSD, CYP17A1, CYP2A, CYP2E1, CYTB5, BAC-CT and/or SULT1A1 gene presented in said sample for a polymorphism; correlating whether a statistically significant association exists between said polymorphism and boar taint in a pig of a particular breed, population or group whereby said pig can be characterized for said polymorphism.

24. An isolated nucleotide sequence which encodes porcine 3αHSD and is as set forth in SEQ ID NO: 66.

25. An isolated nucleotide sequence which encodes porcine CYP2A and is as set forth in SEQ ID NO: 65.

26. A method of identifying a pig which possesses a genotype indicative of a boar taint, said method comprising: obtaining a nucleic acid sample from said pig, and assaying for the presence of a genotype characterized by a polymorphism or haplotype identified within GenBank Accession Number CT171681, said genotype being one which has been shown to be significantly associated with a boar taint trait; and associating said pig with said phenotypic trait based upon the genotype present in said pig.

27. The method of claim 26 wherein said SNP's are at position 166, 523, 707 and/or 745 of GenBank Accession Number CT171681.

28. A method for indirect selection for a polymorphism in an 3αHSD, 3βHSD, CYP17A1, CYP2A, CYP2E1, CYTB5, BAC-CT and/or SULT1A1 gene associated with boar taint comprising: selecting specific alleles of an alternative DNA marker associated with an 3αHSD, 3βHSD, CYP17A1, CYP2A, CYP2E1, CYTB5, BAC-CT and/or SULT1A1 gene, wherein one of the genes is associated with a favorable boar taint trait; making an indirect selection of a polymorphism; and establishing linkage between the specific allele of the alternative DNA and alleles of the DNA marker associated with the boar taint trait.

29. A method for identifying a genetic marker for a favorable boar taint trait in pigs comprising the steps of: breeding male and female pigs of the same breed or breed cross or derived from similar genetic lineages; determining whether the offspring produced have favorable boar taint traits; determining the polymorphism in a 3αHSD, 3βHSD, CYP17A1, CYP2A, CYP2E1, CYTB5, BAG-CT and/or SULT1A 1 gene of each pig; and associating the favorable boar taint traits of offspring produced by pig with said polymorphism thereby identifying a polymorphism for favorable boar taint traits.

30. The method of claim 1 further comprising the step of selecting animals for breeding which are predicted to have favorable boar taint traits by said marker.

31. A method for identifying a marker correlated with favorable boar taint traits comprising the steps of obtaining a sample of genetic material from a pig, said sample comprising a 3αHSD, 3βHSD, CYP17A1, CYP2A, CYP2E1, CYTB5, BAG-CT and/or SULT1A1 gene; assaying said a 3αHSD, 3βHSD, CYP17A1, CYP2A, CYP2E1, CYTB5, BAC-CT and/or SULT1A1 gene presented in said sample for a polymorphism; correlating whether a statistically significant association exists between said polymorphism and favorable boar taint traits in a pig of a particular breed, strain, population, or group whereby said pig can be characterized for said marker.


SEQUENCE LISTING (65 sequences)

SEQ ID NO: 1 - DNA, 20 bp, Sus scrofa
  primer sequence
gtactttgcagaggcactgg

SEQ ID NO: 2 - DNA, 21 bp, Sus scrofa
  primer sequence.
gatttgggataggtgctgatc

SEQ ID NO: 3 - DNA, 24 bp, Sus scrofa
  primer sequence.
gttttgagctgctgaaagatacac

SEQ ID NO: 4 - DNA, 19 bp, Sus scrofa
  primer sequence.
ctggtccagcagagtctgg

SEQ ID NO: 5 - DNA, 19 bp, Sus scrofa
  primer sequence.
tgaccccaaggatatcgac

SEQ ID NO: 6 - DNA, 21 bp, Sus scrofa
  primer sequence.
gcacatctccctcacacttgt

SEQ ID NO: 7 - DNA, 19 bp, Sus scrofa
  primer sequence.
tgaccccaaggatatcgac

SEQ ID NO: 8 - DNA, 21 bp, Sus scrofa
  primer sequence.
gcacatctccctcacacttgt

SEQ ID NO: 9 - DNA, 19 bp, Sus scrofa
  primer sequence.
gactcccactctgttccgc

SEQ ID NO: 10 - DNA, 21 bp, Sus scrofa
  primer sequence.
ccagggtgtaatacttcacgg

SEQ ID NO: 11 - DNA, 20 bp, Sus scrofa
  Primer sequence.
cccaagagtgaagctctgga

SEQ ID NO: 12 - DNA, 20 bp, Sus scrofa
  Primer sequence.
ctctcttcacggtgccatct

SEQ ID NO: 13 - DNA, 20 bp, Sus scrofa
  Primer sequence.
caagtgtgagggagatgtgc

SEQ ID NO: 14 - DNA, 19 bp, Sus scrofa
  Primer sequence.
ttgatttcctatggagccc

SEQ ID NO: 15 - DNA, 21 bp, Sus scrofa
  Primer sequence.
tgagccatggtgttctagaga

SEQ ID NO: 16 - DNA, 21 bp, Sus scrofa
  Primer sequence.
caggcagagggtgatatacgt

SEQ ID NO: 17 - DNA, 21 bp, Sus scrofa
  Primer sequence.
actgttgggatgttgtacagg

SEQ ID NO: 18 - DNA, 21 bp, Sus scrofa
  Primer sequence.
agtacttgatgagagggaccc

SEQ ID NO: 19 - DNA, 21 bp, Sus scrofa
  Primer sequence.
aaaagcttggtcagagaaagc

SEQ ID NO: 20 - DNA, 19 bp, Sus scrofa
  Primer sequence.
agttttgtggcagctctcc

SEQ ID NO: 21 - DNA, 19 bp, Sus scrofa
  Primer sequence.
cgggaaatccttgaaaacc

SEQ ID NO: 22 - DNA, 20 bp, Sus scrofa
  Primer sequence.
agtgtccaaaatgaacccaa

SEQ ID NO: 23 - DNA, 25 bp, Sus scrofa
  Primer sequence.
tttggtagtaatcagagatgaactt

SEQ ID NO: 24 - DNA, 20 bp, Sus scrofa
  Primer sequence.
tgaatttcactccactttgg

SEQ ID NO: 25 - DNA, 20 bp, Sus scrofa
  Primer sequence.
agtgttttctggttcctggc

SEQ ID NO: 26 - DNA, 19 bp, Sus scrofa
  Primer sequence.
ctctgacccagaaaccctc

SEQ ID NO: 27 - DNA, 19 bp, Sus scrofa
  Primer sequence.
acgacacacctccccaaag

SEQ ID NO: 28 - DNA, 20 bp, Sus scrofa
  Primer sequence.
gccagccagtacctcagaga

SEQ ID NO: 29 - DNA, 19 bp, Sus scrofa
  Primer sequence.
tcaggttgctgctatggtg

SEQ ID NO: 30 - DNA, 20 bp, Sus scrofa
  Primer sequence.
aagtggcatcttcctctgaa

SEQ ID NO: 31 - DNA, 19 bp, Sus scrofa
  Primer sequence.
ctcttaggtctccccctcg

SEQ ID NO: 32 - DNA, 20 bp, Sus scrofa
  Primer sequence.
aacttagggctcagacaggc

SEQ ID NO: 33 - DNA, 21 bp, Sus scrofa
  Primer sequence.
ccttttaacctgtttcaccct

SEQ ID NO: 34 - DNA, 19 bp, Sus scrofa
  Primer sequence.
ggcaggtaggcacagagac

SEQ ID NO: 35 - DNA, 21 bp, Sus scrofa
  Primer sequence.
ccttttaacctgtttcaccct

SEQ ID NO: 36 - DNA, 19 bp, Sus scrofa
  Primer sequence.
ggcaggtaggcacagagac

SEQ ID NO: 37 - DNA, 19 bp, Sus scrofa
  Primer sequence.
actgctgtggtccctgtgt

SEQ ID NO: 38 - DNA, 19 bp, Sus scrofa
  Primer sequence.
ttcttcctccagtgatggg

SEQ ID NO: 39 - DNA, 19 bp, Sus scrofa
  Primer sequence.
gtcctcagcacacccacac

SEQ ID NO: 40 - DNA, 19 bp, Sus scrofa
  Primer sequence.
caggtccttagggaagcct

SEQ ID NO: 41 - DNA, 19 bp, Sus scrofa
  Primer sequence.
gtcctcagcacacccacac

SEQ ID NO: 42 - DNA, 19 bp, Sus scrofa
  Primer sequence.
caggtccttagggaagcct

SEQ ID NO: 43 - DNA, 81 bp, Sus scrofa
  Primer sequence.
gtactttgcagaggcactggggccactggagagtttccaagcttggcccgatgaygtgctgatcagcacctatcccaaatc

SEQ ID NO: 44 - DNA, 89 bp, Sus scrofa
  Primer sequence.
gttttgagctgctgaaagatacaccagccccacggctcctcaagacacacttgcccctgrccctgctaccccagactctgctggaccag

SEQ ID NO: 45 - DNA, 108 bp, Sus scrofa
  Primer sequence.
tgaccccaaggatatcgacctcagccccatyrcgattgggtttgccaagattcccccccattacaaactctgtgtcattccccgctcacaagtgtgagggagatgtgc

SEQ ID NO: 46 - DNA, 108 bp, Sus scrofa; identical to SEQ ID NO: 45
  Primer sequence.

SEQ ID NO: 47 - DNA, 109 bp, Sus scrofa
  Primer sequence.
gactcccactctgttccgctcatctctgccgctgtcagcagggcctgaggttcgccgckttacgaaatggccgaacagtccgacaaagccgtgaagtattacaccctgg

SEQ ID NO: 48 - DNA, 142 bp, Sus scrofa
  Primer sequence.
cccaagagtgaagctctggaggccaccaaatatgccatagaagttgggttccgtcayatcgatagtgcttatttataccaaaatgaagagcaggttggacaggccattcgaagcaagattgcagatggcaccgtgaagagag

SEQ ID NO: 49 - DNA, 112 bp, Sus scrofa
  Primer sequence.
caagtgtgagggagatgtgctckaaaggccctggttccttgatgctgacctggaggcctcctgtccccagtgtccccacagggagcgcagcccgggctccataggaaatcaa

SEQ ID NO: 50 - DNA, 110 bp, Sus scrofa
  Primer sequence.
tgagccatggtgttctagagaaataactaaaacacattggaaaggaatttttctaaataacagagcatcrtagatttttataatcaatgacgtatatcaccctctgcctg

SEQ ID NO: 51 - DNA, 112 bp, Sus scrofa
  Primer sequence.
actgttgggatgttgtacaggggaggagagygagctcgcagcatggagccggtccaggacacctaccgcccgccactggagtacgtgaagggggtccctctcatcaagtact

SEQ ID NO: 52 - DNA, 104 bp, Sus scrofa
  Primer sequence.
aaaagcttggtcagagaaagctgggggctgagacaggcaggccctggartagtgatttttttcaagtgcacactggagcacccccggagagctgccacaaaact

SEQ ID NO: 53 - DNA, 104 bp, Sus scrofa
  Primer sequence.
cgggaaatccttgaaaaccgtaaggtaggtggtgatgaagcaggagagatgacgaattaggttgaaagtgtcctgaragcaggcttgggttcattttggacact

SEQ ID NO: 54 - DNA, 110 bp, Sus scrofa
  Primer sequence.
tttggtagtaatcagagatgaacttttttgaaatttgtcaactcttttcctttctcttttcctcccccaytgaatttgccagttgatttcccaaagtggagtgaaattca

SEQ ID NO: 55 - DNA, 101 bp, Sus scrofa
  Primer sequence.
agtgttttctggttcctggcaagtatttctcggygcccaggtttagcaatggctggatggagctgccttgtgacaggaggaggagggtttctgggtcagag

SEQ ID NO: 56 - DNA, 112 bp, Sus scrofa
  Primer sequence.
acgacacacctccccaaagctacgatgacctcaattacacgttgggcaaggartggggcttctgccttgattccagaaggagccttccgccctctctgaggtactggctggc

SEQ ID NO: 57 - DNA, 120 bp, Sus scrofa
  Primer sequence.
tcaggttgctgctatggtgcaggtttgatcccyagtctgggaatttctgcatgccatgggcatggccaaaaataaataaataaaataaaaagagtgtgacttcagaggaagatgccactt

SEQ ID NO: 58 - DNA, 104 bp, Sus scrofa
  Primer sequence.
ctcttaggtctccccctcgctttctccaagacaatctgtgaatccaggtgtcatcatacatrcagccacatgggggcagtgtgggcctgtctgagccctaagtt

SEQ ID NO: 59 - DNA, 114 bp, Sus scrofa
  Primer sequence.
ccttttaacctgtttcaccctccatcaccggaggccaggagaagcmtgggctgagccccttcctcccacagctctgcctctccrcagctttctatgtctctgtgcctacctgcc

SEQ ID NO: 60 - DNA, 114 bp, Sus scrofa; identical to SEQ ID NO: 59
  Primer sequence.

SEQ ID NO: 61 - DNA, 112 bp, Sus scrofa
  misc_feature (28)..(28): R is A or G
actgctgtggtccctgtgtccaatgctcacaccagtctccgcacccgcccgctgctggacttgatctctgcttggcccccagcatrggccaggcccatcactggaggaagaa

SEQ ID NO: 62 - DNA, 114 bp, Sus scrofa
  Primer sequence.
gtcctcagcacacccacacgtcaaatgrgaagcattgatcctaacagtgatgctgctgctgctgctgctgatggaaacggtcccatcaacccagcaggcttccctaaggacctg

SEQ ID NO: 63 - DNA, 114 bp, Sus scrofa
  Primer sequence.
gtcctcagcacacccacacgtcaaatgagaagcattgatcctaacagtgatgctgctgctgctgctgctgatggaaayggtcccatcaacccagcaggcttccctaaggacctg

SEQ ID NO: 64 - DNA, 3499 bp, Sus scrofa
  misc_feature (148)..(148): R = A or G
actatagggcacgcgtggtcgacggcccgggctggtcctacctgatgccaagggcggtgcctactgctgtggtccctgtgtccaatgctcacaccagtctccgcacccgcccgctgctggacttgatctctgcttggcccccagcatrggccaggcccatcactggaggaagaacaaggagagagggttcagatcccagctcctaagcttacctgctccctgcgtgacctccagcaagtggctttagagaggctcctcttctcaactgcaaaatgaagccgatgatggacctgccctgttgtcataaggattcaataaggccacgcatatgtagactcagtcctcacaggcagtgcttcccggggtaaccatcgttctaaaggaagcacatggggtggggagaggacagcagggccacccccctcctttctgcacccacttccagcatcccagggacccctcagttcctgacacaggagtccacccacttctctcttaacatagctccctctgcctgcaaagagcagccccgacaaaccgggaatcacccctaaaggggacttgacaccccctcaaatacaaccttctcttcccaaatgctccctttccatggtgggaaaactcgaccccagaaggcgagtgcaaagcaggagagacagggggcacacgtgtgccccttgcccactctctgtcttctgtcctcagcacacccacacgtcaaatgrgaagcattgatcctaacagtgatgctgctgctgctgctgctgatggaaayggtcccatcaacccagcaggcttccctaaggacctggggagggaaggagcagggccctctgtgagttctgatccttgacacagttgggatttttcagtatcaggctggcggttagtcctgttccccaagccctggccagtccctctgccagctgaaaccatgagttattcttctccagttctgtcaaaggttggacagaaatgcagctctggtcttctaccgcttacccaaccagacctgggcaattctgtgacaccctcctggcctcgctttgaggttccaatgacaattccggggatcaaggggcggcactgtgtccaaaataatagcaggtcaataactggggtcaggtgctaacgccctgatccagctgaactctcttcccagcaacccctcatccacagctctggtcctttctcactgcagcaccctcaaatctattctctagaatcccctccccaggcataagacccttgaatctacctccgttctcactgaaagatccccaaatctgcagccacacatcctgcctcattccaatacccttaaatccaggtctttgaattcttctttcctgagacctcaaaatccacaactttggagtcagttctccctctgagactcccaatccaaagttcaggggttcaccccaaaacaactagtccaaagtcttcagttctgtaacttatctactgccccctccaaagtccaaagccaagactagccccttctgggggaccacaaattccatcttagggcacactccctgttaatctgaactggggtccccctcctccttcctggctggctacgtcccaagctaggcggggagcatcacagggggtgtagttgggaggtgaaatgagacagttatataatcaggaccaaagcctgcccttctctcccaggcggtataaaagcacccatcccaacccatcaccaactgaccgtccctcgcagtgccaccatgctggcctcaggcttgcttctcgtggctctgctgacctgcctgaccataatggtcttgatgtccgtctggcgccagaggaagctccaggggaaactgccccccggacccaccccgctgcccttcatcgggaactacctgcagctgaacacggagcagatgtacaactccctcatgaagatcagccagcgctatggccctgtgttcaccgtccacctggggccccggcggatagtggtgctgtgtggatacgacgcggtgaaggaggccctggtggaccaggctgaggaattcagcgggcgaggcgagcaggccactttcgactggctcttcaaaggctatggcgtggccttcagcaacggcgagcgtgccaagcagctccggcgcttctccatcaccacgctgcgggacttcggcgtgggcaagcggggtatcgaggagcgcatccaggaggaggcgggccacctcatcgaggccttccggggcacgcgcggcgcgttcatcgaccccacctacttcctcagccgaacggtttccaatgtcatcagctccattgtcttcggagaccgctttgactatgaggacaaagagttcctcgcactgctgcggatgatgctgggaagctttcagttcacagctacctctaccggacagctctatgagatgttctactcggtgatgaaacacctgccagggccgcagcaacaggcatttaaggacctgcaggggctggaggacttcatagccaggaaggtggaacacaaccagcgcacgctggatcccaactccccgcgagacttcatcgactccttcctcatccgcatgcaggaggagaagaagaatcctgacaccgagttctattggaagaacctggttctgaccacactgaacctcttcttcgcgggcaccgagacggtcagcacaacgatgcgctacggcttcctgctgctcatgaagaaaccggatgtggaggccaaagtccacgaggagattgaccgcgtgatcggcaggaaccgccaggccaagttcgaggaccgggccaagatgccctacacggaggccgtgatccacgagatccagagattcggagacatgatccccatgggcctggcccgaagagtcaccaaggataccaagtttcgggacttcctcctccccaagggcactgaggtgttccctatgctgggctctgtgctgagagaccccaagttcttctccaacccccgaggcttcaacccccagcacttcctggatgagaacgggcagtttaagaagaatgatgcttttgtgcccttctccatcggaaagcggtactgtttcggagaaggtctggctagaatggagctcttcctcttcctcaccaacatcctgcagaacttccacctcaagtctccgcagctgccccaggacatcgacgtgtcccccaaacacgtgggcttcgccaccatccccccgacctacaccatgagcttccagccccgctgagcccgggctgtgccagggcagggctcgggggaggagcgagggggcgggggcggggagggggcggggctaacgccaggggatgggggacccagggggaagggtggagaggagaggaggaaggaacagaacggaggagctgttcactttactagaaatggagtcttccgaggcccggcgggagggaaagaagacttttcttctttttaagacgatgcttggagtaataacaataacacgttttttttcctaaaaaaaaaaaaaaaaaaaaaaaa

SEQ ID NO: 65 - DNA, 1073 bp, Sus Scrofa
  misc_feature (158)..(158): Y = C or T
cgggagctctggtgatggatcccaaaagccagcgtcttcggcttaacgatggtcacttcattcctgtactgggatttggtacctatgcacctgaagaggttcccaagagtgaagctctggaggccaccaaatatgccatagaagttgggttccgtcayatcgatagtgcttatttataccaaaatgaagagcaggttggacaggccattcgaagcaagattgcagatggcaccgtgaagagagaagacatattctacacgtcaaagctttgggccactttccttcgaccagagttggtccgaccagccttggaaaagtccctgaagaatctccaactggactatgtggatctctatattattcattttccagtggctctgaagcccggggaggaacttttgccaacagatgaaaacggaaaagcactatttgacacagtggatctctgtcgcacgtgggaggccttggagaagtgtaaggacgcaggactgaccaagtccatcggcgtgtccaactttaaccaccaacagctggagaggatcctgaacaagccagggctcaagtacaagcccgtctgcaaccaggtggaatgtcatccttacctcaaccagagcaagcttctggagttttgcaagtccaaggacatcgttctagttgcctatagtgcactgggatcccaaagaaactcaaagtgggtggaagagagcaacccatatctcttagaggatccagtcttaaatgctattgccaagaaacacaacagaagcccagcgcaggttgccctgcgctaccagctgcagcggggagtggtggtcctggccaagagcttcaatgagcagaggatcaaagagaacttccaggtttttgactttgaattgcctccagaagatatgaaaacaatcgatggcctcaaccaaaatttaagatattttaagttactctttgctgtcgatcacccttactacccctattctgaagagtactgagcgggagctctccatcgggtgggctaccagaacctcttgcttctcgggctgtgaagagggtttctgtacttggtagaggtgtttaat